(19) United States
(12) Patent Application Publication
(10) Pub. No.: US 2019/0274953 A1
(43) Pub. Date: Sep. 12, 2019

(54) PH 7 INJECTABLE SOLUTION COMPRISING AT LEAST ONE BASIC INSULIN WITH A PI COMPRISED FROM 5.8 TO 8.5 AND A CO-POLYAMINOACIDE BEARING CARBOXYLATE CHARGES AND HYDROPHOBIC RADICALS

(71) Applicant: ADOCIA, Lyon (FR)

(72) Inventor: Alexandre GEISSLER, Lyon (FR)

(73) Assignee: ADOCIA, Lyon (FR)

(21) Appl. No.: 16/212,960

(22) Filed: Dec. 7, 2018

Related U.S. Application Data
(60) Provisional application No. 62/606,138, filed on Dec. 7, 2017.

(30) Foreign Application Priority Data
Jun. 29, 2018 (FR) 18/56067

(51) Int. Cl. A61K 9/00; A61K 9/08; A61K 38/28; A61K 47/18; A61K 47/22; C07D 207/16

(52) U.S. Cl. CPC A61K 9/0019; A61K 9/08; C07D 207/16; A61K 47/183; A61K 47/22; A61K 38/28

(57) ABSTRACT

A composition is in the form of an injectable aqueous solution, the pH of which is comprised from 6.0 to 8.0, and includes at least: insulin glargine, a co-polyamino acid bearing carboxylate charges and hydrophobic Hy radicals, the co-polyamino acid being made of glutamic or aspartic units and the hydrophobic Hy radicals from following formula I below:

*GpRᵣGpAₐGpC)ₚ Formula I

The composition does not include a basal insulin whose isoelectric point pI is from 5.8 to 8.5. A composition further includes prandial insulin.

The invention relates to insulin injection therapies for treating diabetes.

The invention relates to physically stable compositions in the form of an injectable aqueous solution, the pH of which is comprised from 6.0 to 8.0, comprising at least one basal insulin whose isoelectric point (pI) is comprised from 5.8 to 8.5 and a co-polyamino acid bearing carboxylate charges and hydrophobic radicals.

It relates more particularly to physically stable compositions in the form of an aqueous injectable solution, whose pH is comprised from 6.0 to 8.0, comprising at least one basal insulin whose isoelectric point (pI) is comprised from 5.8 to 8.5 in combination with either a prandial insulin or a gastrointestinal hormone or a prandial insulin and a gastrointestinal hormone, and a co-polyamino acid bearing carboxylate charges and hydrophobic radicals.

Insulin therapy, or diabetes therapy by insulin injection, has made remarkable progress in recent years, thanks in part to the development of new insulins that offer improved correction of patients' blood glucose levels compared to human insulin, and better simulation of physiological activity of the pancreas.

When type II diabetes is diagnosed in a patient, a treatment is implemented gradually. The patient first takes oral antidiabetic drugs (OADs) such as Metformin. When OADs alone are no longer sufficient to regulate glucose levels in the blood, a change in treatment must be made and, depending on the specificities of the patients, different combinations of treatments may be used. For example, the patient may be treated with basal insulin glargine or insulin detemir in addition to OADs, and then, depending on the progress of the disease, basal insulin and prandial insulin therapy.

In addition, today, to ensure the transition from OAD therapies, when they are no longer able to control the level of blood glucose in the blood, to a basal insulin/prandial insulin therapy, the injection of GLP-1 RA analogues is recommended.

GLP-1 RAs for Glucagon-Like Peptide-1 receptor agonists are insulinotropic or incretin peptides, and belong to the family of gastrointestinal hormones (or Gut Hormones) that stimulate insulin secretion when blood glucose levels are too high, for example after a meal.

Gastrointestinal hormones (gut hormones) are also called satiety hormones. They include GLP-1 RA (Glucagon-like peptide-1 receptor agonist) and GIP (Glucose-dependent insulinotropic peptide), oxyntomodulin (a proglucagon derivative), peptide YY, amylin, cholecystokinin, pancreatic polypeptide (PP), ghrelin and enterostatin with peptide or protein structures. They also stimulate insulin secretion in response to glucose and fatty acids and are therefore potential candidates for the treatment of diabetes.

Among these, GLP-1 RAs are those that have provided to date the best result in drug development. They have enabled patients with type II diabetes to lose weight while having better control of their blood sugar.

GLP-1 RA analogs or derivatives have thus been developed mainly to improve their stability.

On the other hand, to meet his daily insulin needs, a diabetic currently has, schematically, two types of insulins with complementary actions: prandial insulins (or so-called rapid-acting insulins) and basal insulins. (or so-called slow-acting insulins).

Prandial insulins enable rapid management (metabolism and/or storage) of the glucose provided during meals and snacks. The patient should inject prandial insulin before each food intake, meaning approximately 2 to 3 injections a day. The most commonly used prandial insulins are: human recombinant insulins, NovoLog® (insulin aspart manufactured by NOVO NORDISK), Humalog® (insulin lispro manufactured by ELI LILLY) and Apidra® (insulin glulisine manufactured by SANOFI).

Basal insulins maintain the patient's glycemic homeostasis outside food intake periods. They act essentially to block the endogenic production of glucose (hepatic glucose). The daily dose of basal insulin generally corresponds to 40-50% of the total daily insulin requirements. Depending on the basal insulin used, this dose is dispensed in 1 or 2 injections distributed regularly over the course of the day. The most commonly used basal insulins include Levemir® (detemir insulin manufactured by NOVO NORDISK) and Lantus® (insulin glargine manufactured by SANOFI).

To be thorough, it is worth noting that NPH (NPH Neutral Protamine Hagedorn insulin; Humuline NPH®, Insulatard®) is the oldest basal insulin. This formulation is the result of the precipitation of human insulin (anionic at neutral pH) with a cation protein, protamine. The microcrystals thus formed are dispersed in an aqueous suspension and slowly dissolved after subcutaneous injection. This slow dissolution ensures prolonged release of insulin. However, this release does not ensure a constant concentration of insulin over time. The release profile is bell-shaped and only lasts between 12 and 16 hours. It is therefore injected twice a day. This NPH basal insulin is much less effective than modern basal insulins, Levemir® and Lantus®. NPH is an intermediate-acting basal insulin.

The NPH principle has evolved with the introduction of rapid analog insulins to result in products called “Premix” providing both rapid action and intermediate action. NovoLog Mix® (NOVO NORDISK) and Humalog Mix® (ELI LILLY) are formulations comprising a rapid analog insulin, Novolog® and Humalog®, partially complexed by protamine. These formulations thus contain analog insulin microcrystals with an intermediate action and part of the insulin remains soluble with rapid action. These formulations have the advantage of a rapid insulin but they also have the defect of NPH, i.e. a duration of action limited to from 12 to 16 hours comprised between and insulin released in a bell shape. However, these products allow the patient to inject himself with an intermediate action basal insulin with a fast acting prandial insulin with a single injection. However, many patients would like to reduce the number of injections they take.

Basal insulins currently on the market may be classified according to the technical solution that allows to obtain extended action and, presently, two approaches are used.

The first, that of detemir insulin, is the in vivo binding to albumin bond. It is an analog, soluble at pH 7, which comprises a fatty acid side chain (tetradecanoyl) attached to position B29 which, in vivo, enables this insulin to associate with albumin. Its prolonged action is mainly due to this affinity for albumin after subcutaneous injection.

However, its pharmacokinetic profile does not make it possible to cover one day, hence, it is most frequently used in two injections a day.

Another insulin soluble at pH 7, is degludec insulin marketed under the name of Tresiba®^{d}. It also includes a lateral fatty acid chain attached to insulin (hexadecandioyl-γ-L-Glu).

The second one, that of insulin glargine, is the precipitation at physiological pH. insulin glargine is a human insulin analog obtained by elongating the C terminal part of the B chain of human insulin by two arginine residues, and by substituting the asparagine residue A21 with a glycine residue (U.S. Pat. No. 5,656,722). The addition of two arginine residues was designed to adjust the pI (isoelectric point) of insulin glargine to physiological pH, and thus make this human insulin analogue insoluble in a physiological medium.

Also, the substitution of A21 has been designed to render insulin glargine stable at acidic pH in order to be formulated as an injectable solution at acidic pH. During subcutaneous injection, the transformation of insulin glargine from an acid pH (pH 4-4.5) to a physiological pH (neutral pH) causes its precipitation under the skin. The slow redissolution of insulin glargine micro-particles ensures a slow and prolonged action.

The hypoglycemic effect of insulin glargine is almost constant over 24 hours which enables most patients to restrict themselves to a single injection a day.

Insulin glargine is currently considered as the most frequently used basal insulin.

However, the necessarily acidic pH of basal insulin formulations, whose isoelectric point is comprised from 5.8 to 8.5, of insulin glargine type, may be a real problem because this acidic pH of the insulin glargine sometimes leads to injection pain in patients and above all, prevents any formulation with other proteins and, in particular, with prandial insulins because the latter not stable at acidic pH. The inability to formulate a prandial insulin at acidic pH is explained by the fact that a prandial insulin undergoes, under these conditions, a secondary deamination reaction in position A21, which makes it impossible to meet the stability requirements applicable to injectable drugs.

To date, in the applications WO 2013/021143 A1, WO 2013/104861 A1, WO 2014/124994 A1 and WO 2014/124993 A1 it has been demonstrated that it is possible to solubilize these insulin glargine-type basal insulins, whose isoelectric point is comprised from 5.8 to 8.5, at neutral pH, while maintaining a solubility difference between the in-vitro medium (the container) and the in-vivo medium (under the skin), regardless of the pH.

Application WO 2013/104861 A1, in particular, describes compositions in the form of an aqueous injectable solution, the pH of which is comprised from 6.0 to 8.0, comprising at least (a) a basal insulin whose isoelectric point pI is comprised from 5.8 to 8.5 and (b) a co-polyamino acid bearing carboxylate charges substituted with hydrophobic radicals.

These compositions of the prior art have the major disadvantage of not being sufficiently stable to meet the specifications applicable to pharmaceutical formulations.

In the examples of the experimental section of the present patent application, it is demonstrated that the compositions described in particular in WO 2013/104861 A1 exhibit unsatisfactory stability over time.

There is, therefore, a need to find a solution which enables the solubilization of a basal insulin whose isoelectric point (pI) is comprised from 5.8 to 8.5 while retaining its basal profile after injection but which also enables it to meet the standard physical stability conditions for insulin-based pharmaceutical products.

Surprisingly the applicant has found that co-polyamino acids bearing carboxylate charges and hydrophobic radicals according to the invention make it possible to obtain compositions in the form of solutions which not only meet the requirements described in WO 2013/104861 A1 but which also are able to confer improved physical stability of said compositions without having to increase the number of excipients used.

These a priori performances which have never been achieved are further retained when the basal insulin whose isoelectric point is comprised from 5.8 to 8.5 is combined in the composition with prandial insulin and/or a gastrointestinal hormone.

Thus, surprisingly, the affinity of the co-polyamino acids according to the invention for insulin glargine has been increased in that it enables the solubilization and stabilization of insulin glargines at an [Hy]/[basal insulin] ratio lower than that of the prior art; these results are further obtained without altering or even improving the propensity of insulin glargine to precipitate, as demonstrated in the experimental section.

This improvement in affinity also makes it possible to limit the level of exposure to these excipients in the context of chronic treatments.

Co-polyamino acids bearing carboxylate charges and hydrophobic radicals Hy according to the invention, exhibit excellent resistance to hydrolysis. This can especially be demonstrated under accelerated conditions, for example by hydrolysis tests at basic pH (pH 12).

In addition, forced oxidation tests, for example of the fenton oxidation type, reveal that the co-polyamino acids bearing carboxylate charges and hydrophobic radicals Hy have a good resistance to oxidation.

The invention thus concerns physically stable compositions in the form of an injectable aqueous solution, whose pH is comprised from 6.0 to 8.0, comprising at least:
a) one basal insulin whose isoelectric point (pI) is comprised from 5.8 to 8.5, and
b) a co-polyamino acid bearing carboxylate charges and at least one hydrophobic radical according to formula I.

In one embodiment, the invention relates to a composition in the form of an injectable aqueous solution, whose pH ranges from 6.0 to 8.0, comprising at least:
a) one basal insulin whose isoelectric point (pI) is comprised from 5.8 to 8.5, and
b) a prandial insulin and
c) a co-polyamino acid bearing carboxylate charges and at least one hydrophobic radical according to formula I.

In one embodiment, the invention relates to a composition in the form of an injectable aqueous solution, whose pH is comprised from 6.0 to 8.0, comprising at least:
a) one basal insulin whose isoelectric point (pI) is comprised from 5.8 to 8.5, and
b) a gastrointestinal hormone and
c) a co-polyamino acid bearing carboxylate charges and at least one hydrophobic radical according to formula I.

In one embodiment, the invention relates to a composition in the form of an injectable aqueous solution, whose pH ranges from 6.0 to 8.0, comprising at least:
a) one basal insulin whose isoelectric point (pI) is comprised from 5.8 to 8.5, and
b) a prandial insulin and a gastrointestinal hormone and
c) a co-polyamino acid bearing carboxylate charges and at least one hydrophobic radical according to formula I.

In one embodiment, the invention relates to a physically stable composition in the form of an injectable aqueous solution, the pH of which is comprised from 6.0 to 8.0, comprising at least:
a) An insulin glargine and
b) a co-polyamino acid bearing carboxylate charges and hydrophobic radicals Hy, said co-polyamino acid consisting of glutamic or aspartic units and said hydrophobic radicals Hy being according to formula I below:

*GpRᵣGpAₐGpC)ₚ Formula I

wherein
GpR is a radical according to formulas II, II′ or II″:

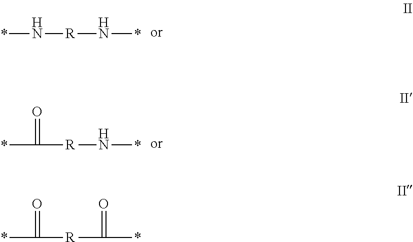

GpA is a radical according to formulas III or III′:

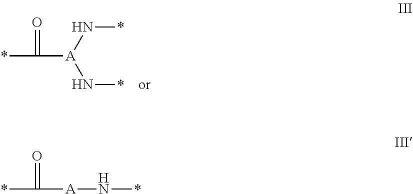

GpC is a radical according to formula IV:

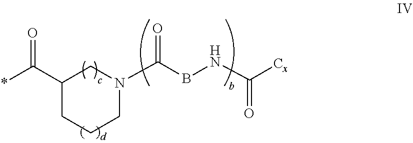

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      through a covalent bond between a nitrogen atom of the hydrophobic radial and a carbonyl of the copolyamino acid, thus forming an amide function resulting from the reaction of an amine function of the precursor of the hydrophobic radical and an acid function borne by the precursor of the co-polyamino acid or
      through a covalent bond between a carbonyl of the hydrophobic radical and a nitrogen atom in N-terminal position of the co-polyamino acid, thus forming an amide function resulting from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or an unsubstituted ether or polyether radical comprising from 4 to 14 carbon atoms and from 1 to 5 oxygen atoms; more precisely, R is a radical chosen from the group consisting of:
   a linear or branched divalent alkyl radical, comprising from 2 to 12 carbon atoms if GpR is a radical according to formula II or from 1 to 11 carbon atoms if GpR is a radical according to formula II′ or II″;
   a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
   an unsubstituted ether or polyether radical comprising from 4 to 14 carbon atoms and from 1 to 5 oxygen atoms;
A is a linear or branched alkyl radical comprising from 1 to 8 carbon atoms and optionally substituted by a radical resulting from a saturated, unsaturated or aromatic ring;
B is a radical chosen from the group consisting of an unsubstituted ether or polyether radical comprising from 4 to 14 carbon atoms and from 1 to 5 oxygen atoms or a linear or branched alkyl radical, optionally comprising an aromatic ring, comprising 1 to 9 carbon atoms;
Cₓ is a linear or branched monovalent alkyl radical, optionally comprising a cyclic part, wherein x indicates the number of carbon atoms and:
   if p is equal to 1, x is comprised from 9 to 25 (9≤x≤25):
   if p is equal to 2, x is comprised from 9 to 15 (9≤x≤15),
the ratio i between the number of hydrophobic radicals and the number of glutamic or aspartic units being between 0<i≤0.5;
when several hydrophobic radicals are carried by a co-polyamino acid they are therefore, identical or different;
the degree of polymerization DP of glutamic or aspartic units is comprised from 5 to 250;
the free acid functions being in the form of an alkaline cation salt chosen from the group consisting of Na⁺ and K⁺,
the said composition comprising at least one ion species chosen from the group of anions, cations and/or zwitterions.

In one embodiment, the invention relates to a physically stable composition in the form of an injectable aqueous solution, the pH of which is comprised from 6.0 to 8.0, comprising at least:
c) An insulin glargine and
d) a co-polyamino acid bearing carboxylate charges and hydrophobic radicals Hy, said co-polyamino acid consisting of glutamic or aspartic units and said hydrophobic radicals Hy being according to formula I below:

*GpRᵣGpAₐGpC)ₚ Formula I

wherein
GpR is a radical according to formulas II, II′ or II″:

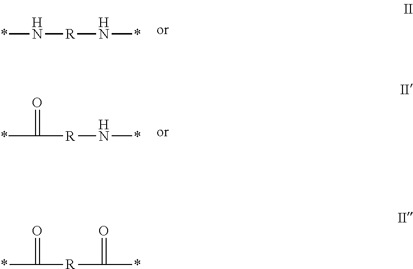

GpA is a radical according to formulas III or III′:

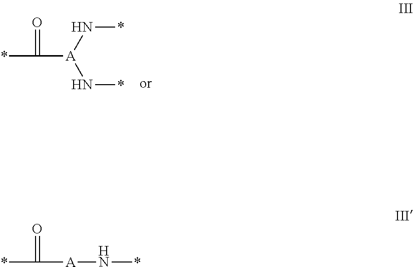

GpC is a radical according to formula IV:

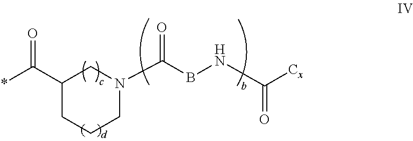

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      through a covalent bond between a nitrogen atom of the hydrophobic radial and a carbonyl of the copolyamino acid, thus forming an amide function originating from the reaction of an amine function of the precursor of the hydrophobic radical and an acid function borne by the precursor of the co-polyamino acid or
      through a covalent bond between a carbonyl of the hydrophobic radical and a nitrogen atom in N-terminal position of the co-polyamino acid, thus forming an amide function originating from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms; more precisely, R is a radical chosen from the group consisting of:
   a linear or branched divalent alkyl radical, comprising from 2 to 12 carbon atoms if GpR is a radical according to formula II or from 1 to 11 carbon atoms if GpR is a radical according to formula II′ or II″;
   a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
   an unsubstituted ether or polyether radical comprising from 4 to 14 carbon atoms and from 1 to 5 oxygen atoms;
A is a radical chosen from the group consisting of an unsubstituted ether or polyether radical comprising from 4 to 14 carbon atoms and from 1 to 5 oxygen atoms or a linear or branched alkyl radical comprising from 1 to 8 carbon atoms and optionally substituted by a radical derived from a saturated, unsaturated or aromatic ring;
B is a linear or branched alkyl radical, optionally comprising an aromatic ring, comprising 1 to 9 carbon atoms;
Cₓ is a linear or branched monovalent alkyl radical, optionally comprising a cyclic part, wherein x indicates the number of carbon atoms and:
   if p is equal to 1, x is comprised from 9 to 25 (9≤x≤25):
   if p is equal to 2, x is comprised from 9 to 15 (9≤x≤15),
the ratio i between the number of hydrophobic radicals and the number of glutamic or aspartic units being between 0<i≤0.5;
when several hydrophobic radicals are carried by a co-polyamino acid they are therefore, identical or different;
the degree of polymerization DP of glutamic or aspartic is comprised from 5 to 250;
the free acid functions being in the form of an alkaline cation salt chosen from the group consisting of Na⁺ and K⁺,
   the said composition comprising at least one ion species chosen from the group of anions, cations and/or zwitterions.

In one embodiment, the invention relates to a physically stable composition in the form of an injectable aqueous solution, the pH of which is comprised from 6.0 to 8.0, comprising at least:
a) an insulin glargine,
b) a prandial insulin and/or a gastrointestinal hormone and
c) a co-polyamino acid bearing carboxylate charges and hydrophobic radicals Hy, said co-polyamino acid being constituted by glutamic or aspartic units and said hydrophobic radicals Hy being according to formula I below:

*GpRᵣGpAₐGpC)ₚ Formula I

wherein
GpR is a radical according to formula II or II′:

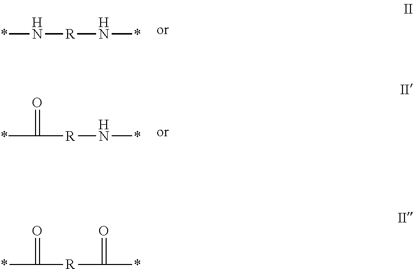

GpA is a radical according to formulas II or III′:

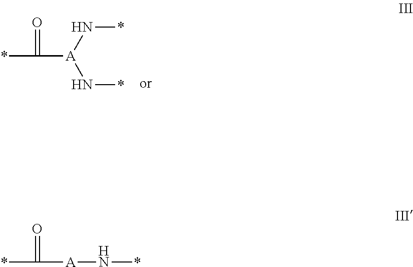

GpC is a radical according to formula IV:

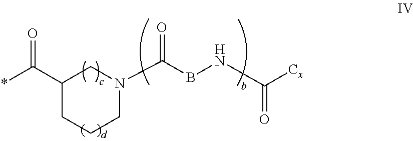

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      through a covalent bond between a nitrogen atom of the hydrophobic radial and a carbonyl of the copolyamino acid, thus forming an amide function originating from the reaction of an amine function of the precursor of the hydrophobic radical and an acid function borne by the precursor of the co-polyamino acid or
      through a covalent bond between a carbonyl of the hydrophobic radical and a nitrogen atom in N-terminal position of the co-polyamino acid, thus forming an amide function originating from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms:
   a linear or branched divalent alkyl radical, comprising from 2 to 12 carbon atoms if GpR is a radical according to formula II or from 1 to 11 carbon atoms if GpR is a radical according to formula II′ or II″;
   a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
   an unsubstituted ether or polyether radical comprising from 4 to 14 carbon atoms and from 1 to 5 oxygen atoms;
A is a linear or branched alkyl radical comprising from 1 to 8 carbon atoms and optionally substituted by a radical resulting from a saturated, unsaturated or aromatic ring;
B is a radical chosen from the group consisting of an unsubstituted ether or polyether radical comprising from 4 to 14 carbon atoms and from 1 to 5 oxygen atoms or a linear or branched alkyl radical, optionally comprising an aromatic ring, comprising 1 to 9 carbon atoms;
Cₓ is a linear or branched monovalent alkyl radical, optionally comprising a cyclic part, wherein x indicates the number of carbon atoms and:
   if p is equal to 1, x is comprised from 9 to 25 (9≤x≤25):
   if p is equal to 2, x is comprised from 9 to 15 (9≤x≤15),
the ratio i between the number of hydrophobic radicals and the number of glutamic or aspartic units being between 0<i≤0.5;
when several hydrophobic radicals are carried by a co-polyamino acid they are therefore, identical or different;
the degree of polymerization DP of glutamic or aspartic units is comprised from 5 to 250;
the free acid functions being in the form of an alkaline cation salt chosen from the group consisting of Na⁺ and K⁺;

In one embodiment, when r=2, then the GpR group bound to the PLG is selected from GpR according to formula II.

In one embodiment, when r=2 then the GpR group bound to the PLG is selected from GpR according to formula II and the second GpR is selected from GpR according to formula II″.

In one embodiment, when r=2, then the GpR group bound to the PLG is selected from GpR according to formula II″.

In one embodiment, when r=2 then the GpR group bound to the PLG is selected from GpR according to formula II″ and the second GpR is selected from GpR according to formula II.

Cₓ is a linear or branched monovalent alkyl radical, wherein x indicates the number of carbon atoms and:
if p is equal to 1, x is comprised from 11 and 25 (11≤x≤25);
if p is equal to 2, x is comprised from 9 and 15 (9≤x≤15).

Said co-polyamino acid bearing carboxylate charges and hydrophobic radicals Hy is soluble in aqueous solution at a pH between 6.0 and 8.0, at a temperature of 25° C. and at a concentration of less than 100 mg/ml.

According to the invention, compositions in the form of an injectable aqueous solution are clear solutions.

By “clear solution” is meant compositions which satisfy the criteria described in the American and European pharmacopoeias concerning injectable solutions. In the American pharmacopoeia, the solutions are defined in section <1151> referring to the injection (<1>) (referring to <788> according to USP 35 and specified in <788> according to USP 35 and in <787>, <788> and <790>USP 38 (from August 1 2014), according to USP 38). In the European pharmacopoeia, injectable solutions have to meet the criteria given in sections 2.9.19 and 2.9.20.

“Physically stable composition” refers to compositions which meet the visual inspection criteria described in the European pharmacopoeia, the American pharmacopoeia and the international pharmacopoeia, i.e. compositions which are clear and which contain no visible particles but also colorless.

By “co-polyamino acid consisting of glutamic or aspartic units” is meant non-cyclic linear sequences of glutamic acid or aspartic acid units bound together by peptide bonds, said sequences having a C-terminal part, corresponding to the carboxylic acid at one end, and an N-terminal portion, corresponding to the amine at the other end of the chain.

By “soluble” is meant, suitable for the preparation of a clear and particle-free solution at a concentration of less than 100 mg/ml in distilled water at 25° C.

By “solution” is meant a liquid composition free of visible particles, using the method according to the European pharmacopoeia 8.0, point 2.9.20, and the American pharmacopoeia.

By “chemically stable composition” is meant compositions which, after storage for a certain time and at a certain temperature, have a minimum recovery of the active ingredients and which comply with the specifications applicable to pharmaceutical products.

“Injectable aqueous solution” refers to water-based solutions which meet the requirements of the European and American pharmacopoeias and which are sufficiently liquid to be injected.

“Alkyl radical” refers to a linear or branched carbon chain which does not comprise a heteroatom.

Co-polyamino acid is a statistical or block co-polyamino acid.

Co-polyamino acid is a statistical co-polyamino acid bound to glutamic and/or aspartic units.

In one embodiment, the composition according to the invention is characterized in that Hy comprises between 15 and 100 carbon atoms.

In one embodiment, the composition according to the invention is characterized in that Hy comprises between 30 and 70 carbon atoms.

In one embodiment, the composition according to the invention is characterized in that Hy comprises between 40 and 60 carbon atoms.

In one embodiment, the composition according to the invention is characterized in that Hy comprises between 20 and 30 carbon atoms

In one embodiment, Hy comprises more than 15 carbon atoms.

In one embodiment, Hy comprises more than 30 carbon atoms.

In one embodiment, the composition is characterized in that the pH is comprised from 6.0 to 8.0.

In one embodiment, the composition is characterized in that the pH is comprised from 6.6 to 7.8.

In one embodiment, the composition is characterized in that the pH is comprised from 7.0 to 7.8.

In one embodiment, the composition is characterized in that the pH is comprised from 6.8 to 7.4.

In formulas, the * indicate the attachment sites of the various elements represented.

In the formulas, the * indicate the attachment sites of the hydrophobic radicals to the co-polyamino acid. The radicals—Hy are attached to the co-polyamino acid through amide functions.

In formulas II and II′ or II″, the * indicate the attachment sites of GpR:

to the co-polyamino acid and

to GpA if a=1 or to GPC if a=0.

In formulas III and III′, the * indicate, from left to right respectively, the attachment sites of GpA:

to GpR if r=1 or 2 or to the co-polyamino acid if r=0 and

to GpC.

In formula IV, the * indicates the attachment sites of GpC:

to GpA if a=1, GpR if r=1 or 2 and a=0 or,

to the co-polyamino acid if r=0 and a=0.

All attachments between the different groups GpR, GpA and GpC are amide functions.

Radicals—Hy, GpR, GpA, and GpC, and D are each independently identical or different from one monomeric unit to another.

In one embodiment, the composition is characterized in that the said hydrophobic radicals are selected from the hydrophobic radicals according to formula I wherein if p is equal to 1 (p=1) and if x is less than or equal to 14 (x≤14) then r=0 or r=1.

In one embodiment, the composition is characterized in that said hydrophobic radicals are selected from hydrophobic radicals according to formula I wherein, if p is equal to 1 (p=1) and if x is comprised from 15 to 16 (15≤x≤16), then r=1.

In one embodiment, the composition is characterized in that the said hydrophobic radicals are selected from the hydrophobic radicals according to formula I wherein if p is equal to 1 (p=1) and if x is greater than 17 (17≤x) then r=1 and R is an ether or polyether radical.

In one embodiment, the composition is characterized in that the said hydrophobic radicals are selected from the hydrophobic radicals according to formula I wherein, if p is equal to 1 (p=1) then x is comprised from 17 to 25 (17≤x≤25).

In one embodiment, at least one hydrophobic radical -Hy is selected from the radicals according to formula I wherein r=2 according to formula Ic′, as defined below:

*-GpR₁-GpR-(GpA)ₐ-(GpC)ₚ Formula Ic′

wherein GpR₁ is a radical according to formula II.

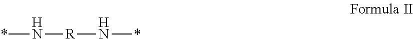

wherein GpR, GpA, GpC, R, a and p have the definitions given above.

In one embodiment, at least one hydrophobic radical -Hy is selected from the radicals according to formula I wherein r=2 according to formula Ic′, as defined below:

*-GpR₁-GpR-(GpA)ₐ-(GpC)ₚ Formula Ic′

wherein GpR₁ is a radical according to formula II″.

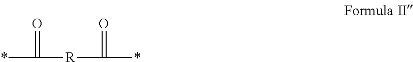

wherein GpR, GpA, GpC, R, a and p have the definitions given above.

In one embodiment, the composition is characterized in that the said hydrophobic radicals are selected from the hydrophobic radicals according to formula I wherein p=1, represented by formula V below:

*-GpRᵣGpAₐGpC Formula V

GpR, GpA, GpC, r and a have the definitions given above.

In one embodiment, the composition is characterized in that the said hydrophobic radicals are selected from the hydrophobic radicals according to formula I wherein a=1 and p=2, represented by formula VI below:

*GpRᵣGpAGpC)₂ Formula VI

wherein

GpR, GpA, GpC and r have the definitions given above.

In one embodiment, the composition is characterized in that the hydrophobic radical is a radical according to formula I, V or VI wherein: r is equal to 1 (r=1) and a is equal to 0 (a=0).

In one embodiment, the composition is characterized in that the hydrophobic radical is a radical according to formula I, Ic′, V or VI wherein: r is equal to 2 (r=2) and a is equal to 0 (a=0).

In one embodiment, the composition is characterized in that the hydrophobic radical is a radical according to formula I, V or VI wherein r is equal to 1 (r=1) and a is equal to 1 (a=11).

In one embodiment, the composition is characterized in that the hydrophobic radical is a radical according to formula I, Ic′, V or VI wherein r is equal to 2 (r=2) and a is equal to 1 (a=1).

In one embodiment, the composition is characterized in that the hydrophobic radical is a radical according to formula I, V or VI wherein a is equal to 0 (a=0) and r is equal to 0 (r=0).

In one embodiment, the composition is characterized in that the hydrophobic radical is a formula V radical wherein r=1, a=1, GpR corresponds to formula II, GpA corresponds to formula III′ wherein A is

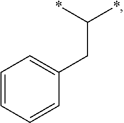

GpC corresponds to formula IVd.

In one embodiment, the composition is characterized in that the hydrophobic radical is a formula V radical wherein r=1, a=1, GpR corresponds to formula I wherein R is a linear divalent alkyl, GpA corresponds to formula III′ wherein A is

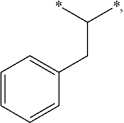

GpC corresponds to formula IVd.

In one embodiment, the composition is characterized in that the hydrophobic radical is a formula V radical wherein r=1, a=1, GpR corresponds to formula II wherein R is —CH2—CH2-, GpA corresponds to formula III′ wherein A is

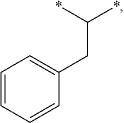

GpC corresponds to formula IVd

In one embodiment, the composition is characterized in that the hydrophobic radical is a formula V radical wherein r=1, a=1, GpR corresponds to formula II wherein R is —CH2—CH₂—, GpA corresponds to formula III′ wherein A is

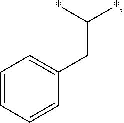

GpC corresponds to formula IVd wherein x=13 and Cx is

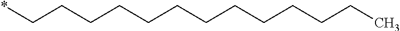 [SMILES: *CCCCCCCCCCCCC]

In one embodiment the composition is characterized in that the hydrophobic radical is a radical according to formula I, Ic′, V or VI wherein GpR is a radical according to formula II.

In one embodiment, the composition is characterized in that the hydrophobic radical is a radical according to formula I, Ic′, V or VI wherein GpR is a radical according to formula II wherein R is a divalent alkyl radical comprising from 2 to 12 carbon atoms.

In one embodiment, the composition is characterized in that the hydrophobic radical is a radical according to formula I, Ic′, V or VI wherein GpR is a radical according to formula II wherein R is a divalent alkyl radical comprising from 2 to 6 carbon atoms.

In one embodiment, the composition is characterized in that the hydrophobic radical is a radical according to formula I, Ic′, V or VI wherein GpR is a radical according to formula II wherein R is a divalent alkyl radical comprising from 2 to 6 carbon atoms.

In one embodiment, the composition is characterized in that the hydrophobic radical is a radical according to formula I, Ic′, V or VI wherein GpR is a radical according to formula II wherein R is a divalent alkyl radical comprising from 2 to 4 carbon atoms.

In one embodiment, the composition is characterized in that the hydrophobic radical is a radical according to formula I, Ic′, V or VI wherein GpR is a radical according to formula II wherein R is a divalent alkyl radical comprising from 2 to 4 carbon atoms.

In one embodiment, the composition is characterized in that the hydrophobic radical is a radical according to formula I, Ic′, V or VI wherein GpR is a radical according to formula II wherein R is a divalent alkyl radical comprising 2 carbon atoms.

In one embodiment the composition is characterized in that the hydrophobic radical is a radical according to formula I, Ic′, V or VI wherein GpR is a radical according to formula II′.

In one embodiment, the composition is characterized in that the hydrophobic radical is a radical according to formula I, Ic′, V or VI wherein GpR is a radical according to formula II wherein R is a linear divalent alkyl radical comprising from 1 to 11 carbon atoms.

In one embodiment, the composition is characterized in that the hydrophobic radical is a radical according to formula I, Ic′, V or VI wherein GpR is a radical according to formula II wherein R is a divalent alkyl radical comprising from 1 to 6 carbon atoms.

In one embodiment, the composition is characterized in that the hydrophobic radical is a radical according to formula I, Ic′, V or VI wherein GpR is a radical according to formula II or II′ wherein R is a divalent alkyl radical comprising from 2 to 5 carbon atoms and having one or more amide functions (—CONH₂).

In one embodiment, the composition is characterized in that the hydrophobic radical is a radical according to formula I, Ic′, V or VI wherein GpR is a radical according to formula II′ or II wherein R is a linear divalent alkyl radical comprising from 2 to 5 carbon atoms and having one or more amide functions (—CONH₂).

In one embodiment, the composition is characterized in that the hydrophobic radical is a radical according to formula I, Ic′, V or VI wherein GpR is a radical according to formula II or II′ wherein R is a radical chosen from the group consisting of radicals represented by the formulas below:

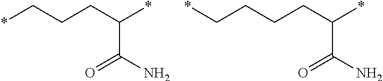

In one embodiment, the composition is characterized in that the R radical is bound to the co-polyamino acid through an amide function carried by the carbon in the delta or epsilon position (or in position 4 or 5) relative to the function amide (—CONH₂).

In one embodiment, the composition is characterized in that the hydrophobic radical is a radical according to formula I, Ic′, V or VI wherein GpR is a radical according to formula II, II′ or II″, wherein R is an unsubstituted linear ether or polyether radical comprising from 4 to 14 carbon atoms and from 1 to 5 oxygen atoms.

In one embodiment, the composition is characterized in that the hydrophobic radical is a radical according to formula I, Ic′, V or VI wherein GpR is a radical according to formula II, II′ or II″, wherein R is an ether radical.

In one embodiment, the composition is characterized in that the hydrophobic radical is a radical according to formula I, Ic′, V or VI wherein GpR is a radical according to formula II, II′ or II″, wherein R is an ether radical comprising from 4 to 6 carbon atoms.

In one embodiment, the composition is characterized in that the hydrophobic radical is a radical according to formula I, Ic′, V or VI wherein GpR is a radical according to formula II, II′ or II″ wherein R is an ether radical represented by formula

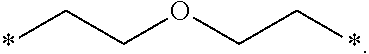

In one embodiment the composition is characterized in that the hydrophobic radical is a radical according to formula I, Ic′, V or VI wherein GpR is a radical according to formula II, II′ or II″ wherein R is a polyether radical.

In one embodiment, the composition is characterized in that the hydrophobic radical is a radical according to formula I, Ic′, V or VI wherein GpR is a radical according to formula II, II′ or II″, wherein R is a linear polyether radical comprising from 6 to 10 carbon atoms and from 2 to 3 oxygen atoms.

In one embodiment, the composition is characterized in that the hydrophobic radical is a radical according to formula I, Ic′, V or VI wherein GpR is a radical according to formula II, II′ or II″ wherein R is a polyether radical chosen from the group consisting of the radicals represented by the formulas below:

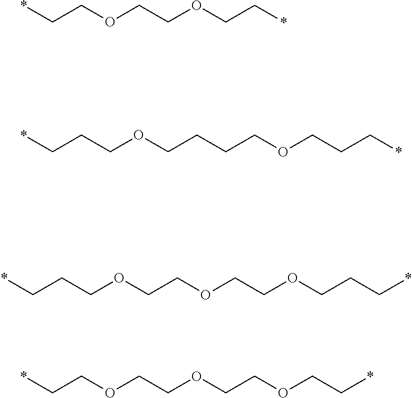 [SMILES: *CCOCCOCC* ; *CCCOCCCCOCCC* ; *CCCOCCOCCOCCC* ; *CCOCCOCCOCC*]

In one embodiment, the composition is characterized in that the hydrophobic radical according to formula I, Ic′, V or VI wherein GpR is a radical according to formula II wherein R is a polyether radical chosen from the group consisting of the radicals represented by the formulas below:

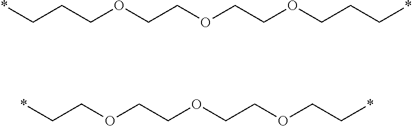 [SMILES: *CCCOCCOCCOCCC* ; *CCOCCOCCOCC*]

In one embodiment, the composition is characterized in that the hydrophobic radical is a radical according to formula I, Ic′, V or VI wherein a is equal to 1 (a=1) and the GpA radical is a radical according to formula III′ wherein A is chosen from the group consisting of the radicals represented by the formulas below:

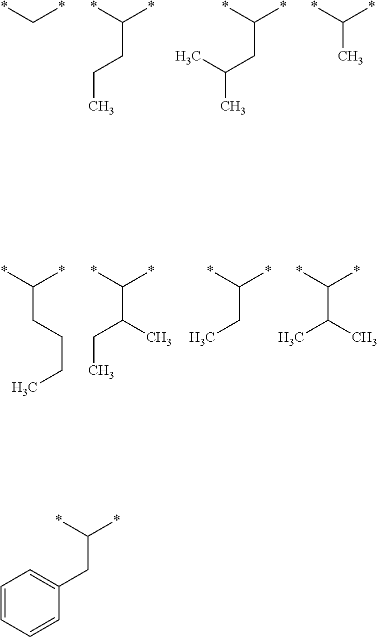

In one embodiment, the composition is characterized in that the hydrophobic radical is a radical according to formula I, Ic′, V or VI wherein a is equal to 1 (a=1) and the GpA radical is a radical according to formula III wherein A is chosen from the group consisting of the radicals represented by the formulas below:

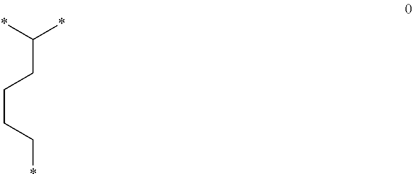 [SMILES: *CCCCC(*)*]

In one embodiment, the composition is characterized in that the hydrophobic radical is a radical according to formula I, Ic′, V or VI wherein the GpC radical according to formula IV is chosen from the group consisting of the radicals according to formulas IVa, IVb or IVc hereinafter represented:

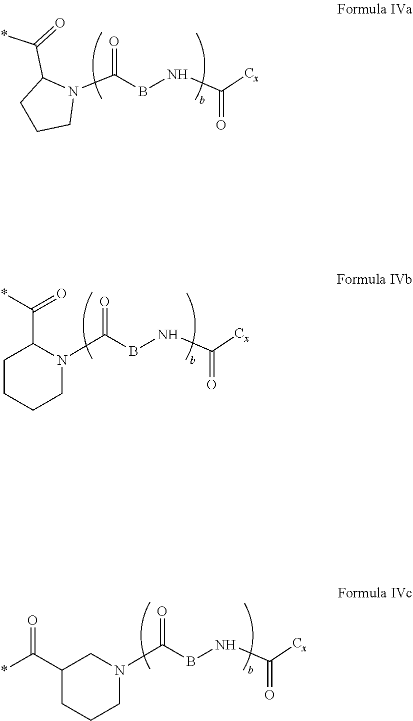

In one embodiment, the composition is characterized in that the hydrophobic radical is a radical according to formula I, Ic′, V or VI wherein the GpC radical is according to formula IVa.

In one embodiment, the composition is characterized in that the hydrophobic radical is a radical according to formula I, Ic′, V or VI wherein the GpC radical according to formula IV is chosen from the group consisting of the radicals according to formulas IVa, IVb or IVc wherein b is equal to 0, respectively corresponding to formulas IVd, IVe, and IVf hereinafter represented:

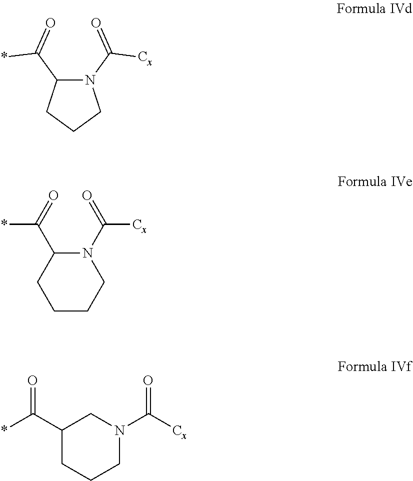

In one embodiment, the composition is characterized in that the hydrophobic radical is a radical according to formula I, Ic′, V or VI wherein the GpC radical corresponds to formula IV or IVa wherein b=0, and corresponds to formula IVd.

In one embodiment, the composition is characterized in that the hydrophobic radical is a radical according to formula I, Ic′, V or VI wherein the GpC radical according to formula IV wherein b=1 is chosen from the group consisting of radicals wherein B is a radical residue of amino acid chosen from the group consisting of the radicals represented by the formulas below:

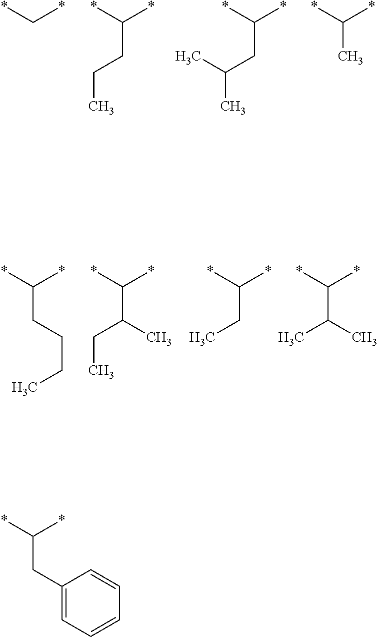

In one embodiment, the composition is characterized in that the hydrophobic radical is a radical according to formula I, Ic′, V or VI wherein the GpC radical according to formula IV or IVa wherein b=1 is chosen from the group consisting of radicals wherein B is an amino acid residue chosen from the group consisting of the radicals represented by the formulas below:

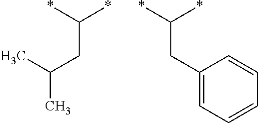

In one embodiment, the composition is characterized in that the hydrophobic radical is a radical according to formula I, Ic′, V or VI wherein the GpC radical according to formula IV is chosen from the group consisting of radicals wherein Cx is chosen from the group consisting of linear alkyl radicals.

In one embodiment, the composition is characterized in that the hydrophobic radical is a radical according to formula I Ic′, V or VI wherein the GpC radical according to formula IV is chosen from the group consisting of radicals wherein Cx is chosen from the group consisting of branched alkyl radicals.

In one embodiment, the composition is characterized in that the hydrophobic radical is a radical according to formula I, Ic′, V or VI wherein the GpC radical according to formula IV is chosen from the group consisting of radicals wherein Cx is chosen from the group consisting of alkyl radicals comprising from 11 to 14 carbon atoms.

In one embodiment, the composition is characterized in that the hydrophobic radical is a radical according to formula I, Ic′, V or VI wherein the GpC radical according to formula IV is chosen from the group consisting of radicals wherein Cx is chosen from the group consisting of linear alkyl radicals:

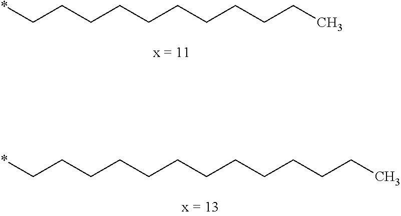 [SMILES: *CCCCCCCCCCC ; *CCCCCCCCCCCCC]

In one embodiment, the composition is characterized in that the hydrophobic radical is a radical according to formula I, Ic′, V or VI wherein the GpC radical according to formula IV is chosen from the group consisting of radicals wherein Cx is chosen from the group consisting of alkyl radicals comprising from 15 to 16 carbon atoms.

In one embodiment, the composition is characterized in that the hydrophobic radical is a radical according to formula I, V or VI wherein the GpC radical according to formula IV is chosen from the group consisting of radicals wherein Cx is chosen from the group consisting of linear alkyl radicals:

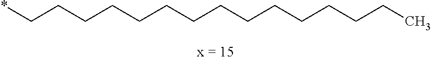 [SMILES: *CCCCCCCCCCCCCCC]

In one embodiment, the composition is characterized in that the hydrophobic radical is a radical according to formula I, Ic′, V or VI wherein the GpC radical according to formula IV is chosen from the group consisting of radicals wherein Cx is chosen from the group consisting of linear alkyl radicals:

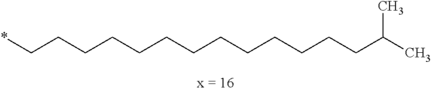 [SMILES: *CCCCCCCCCCCCCC(C)C]

In one embodiment, the composition is characterized in that the hydrophobic radical is a radical according to formula I, Ic′, V or VI wherein the GpC radical according to formula IV is chosen from the group consisting of radicals wherein Cx is chosen from the group consisting of alkyl radicals comprising from 17 to 25 carbon atoms.

In one embodiment, the composition is characterized in that the hydrophobic radical is a radical according to formula I, Ic′, V or VI wherein the GpC radical according to formula IV is chosen from the group consisting of radicals wherein Cx is chosen from the group consisting of alkyl radicals comprising from 17 to 18 carbon atoms.

In one embodiment, the composition is characterized in that the hydrophobic radical is a radical according to formula I, Ic′, V or VI wherein the GpC radical according to formula IV is chosen from the group consisting of radicals wherein Cx is chosen from the group consisting of linear alkyl radicals represented by the formula below:

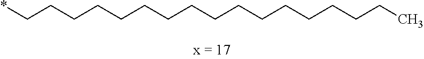 [SMILES: *CCCCCCCCCCCCCCCCC]

In one embodiment, the composition is characterized in that the hydrophobic radical is a radical according to formula I, Ic′, V or VI wherein the GpC radical according to formula IV is chosen from the group consisting of radicals wherein Cx is chosen from the group consisting of alkyl radicals comprising from 18 to 25 carbon atoms.

In one embodiment, the composition is characterized in that the hydrophobic radical is a radical according to formula I, Ic′, V or VI wherein the GpC radical according to formula IV is chosen from the group consisting of radicals wherein Cx is chosen from the group consisting of linear alkyl radicals represented by the formulas below:

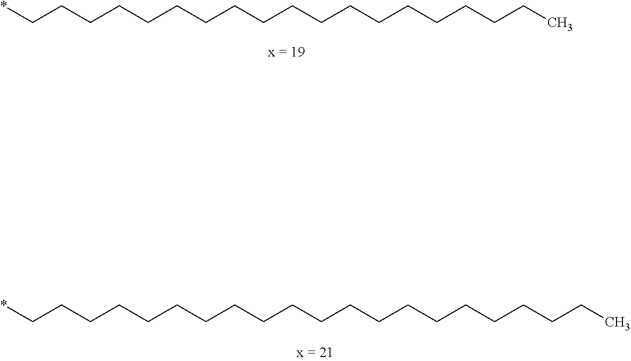 [SMILES: *CCCCCCCCCCCCCCCCCCC ; *CCCCCCCCCCCCCCCCCCCCC]

In one embodiment, the composition is characterized in that the co-polyamino acid bearing carboxylate charges and hydrophobic radicals is selected from the co-polyamino acids according to formula VII below:

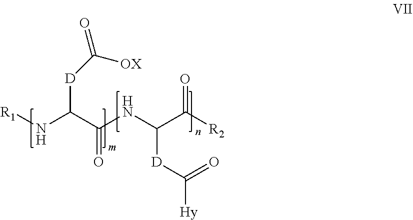

wherein,
D represents, independently, either a —CH₂— group (aspartic unit) or a —CH₂—CH₂— group (glutamic unit);
Hy is a hydrophobic radical selected from hydrophobic radicals according to formula I, V or VI;
R₁ is a hydrophobic radical selected from hydrophobic radicals according to formulas I, V or VI, or a radical chosen from the group consisting of a H, a C2 to C10 linear acyl group, a C3 to C10 branched acyl group, benzyl, a terminal “amino acid” unit and a pyroglutamate;
R₂ is a hydrophobic radical selected from the hydrophobic radical according to formula I, V or VI wherein r=1 and GpR is a radical according to formula II or a radical —NR′R″ radical, R′ and R″, an identical or different, chosen from the group consisting of H, linear or branched of cyclical C2 to C10 alkyls, benzyl, and the said R′ and R″ alkyls could form together one or more saturated, unsaturated and/or aromatic carbon cycles and/or could contain heteroatoms, chosen from the group consisting of O, N and S;
X represents a H or a cationic entity chosen from the group consisting of metal cations;
n+m represents the degree of polymerization DP of the co-polyamino acid, namely the average number of monomeric units per co-polyamino acid chain and 5≤n+m≤250.

The co-polyamino acid bearing carboxylate charges and at least one hydrophobic radical according to formula I can also be referred to as “co-polyamino acid” in the present description.

A “statistical co-polyamino acid” refers to a co-polyamino acid bearing carboxylate charges and at least one hydrophobic radical, a co-polyamino acid according to formula VIIa.

In one embodiment, the composition is characterized in that the co-polyamino acid bearing carboxylate charges and hydrophobic radicals is selected from the co-polyamino acids according to formula VII wherein R₁=R′₁ and R₂=R′₂, according to formula VIIa below:

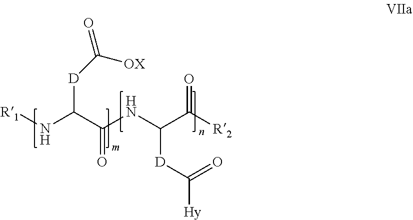

wherein,
m, n, X, D and Hy have the definitions provided above;
R′₁ is a radical chosen from the group consisting of H, linear C2 to C10 acyl group, branched C3 to C10 acyl group, benzyl, terminal amino acid unit and pyroglutamate;

R′₂ is a radical —NR′R″, R′ and R″ identical or different, being chosen from the group consisting of H, linear or branched or cyclic C2 to C10 alkyls, benzyl and said R′ and R″ alkyls which can form together one or more saturated, unsaturated and/or aromatic carbon rings and/or which may comprise heteroatoms chosen from the group consisting of O, N and S. In one embodiment, the composition is characterized in that the co-polyamino acid bearing carboxylate charges and hydrophobic radicals is chosen from the co-polyamino acids according to formulas VIIa wherein Hyd is according to formula V, GpR is according to formula II, GpA is according to formula III′ wherein A is the radical

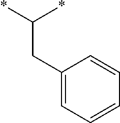 [SMILES: *C(*)Cc1ccccc1]

and GpC corresponds to formula IVd.

In one embodiment the composition is characterized in that the co-polyamino acid bearing carboxylate charges and hydrophobic radicals is selected from the formula VIIa co-polyamino acids wherein Hyd has formula V, GpR has formula II wherein R is —CH2—CH2-, GpA is according to formula III′ wherein A is the radical

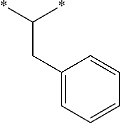 [SMILES: *C(*)Cc1ccccc1]

and GpC corresponds to formula IVd.

A “defined co-polyamino acid” refers to a co-polyamino acid bearing carboxylate charges and at least one hydrophobic radical, a co-polyamino acid according to formula VIIb.

In one embodiment, the composition is characterized in that the co-polyamino acid bearing carboxylate charges and hydrophobic radicals is selected from the co-polyamino acids according to formula VII wherein n=0 according to formula VIIb below:

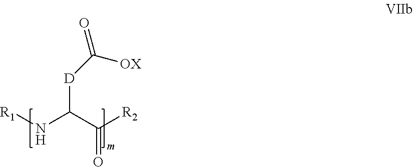

wherein m, X, D, R₁ and R₂ have the definitions given above and at least R₁ or R₂ is a hydrophobic radical according to formula I, V or VI.

In one embodiment, the composition is characterized in that the co-polyamino acid bearing carboxylate charges and hydrophobic radicals is selected from the co-polyamino acids according to formula VII wherein n=0 according to formula VIIb and R₁ or R₂ is a hydrophobic radical according to formula I, V or VI.

In one embodiment, the composition is characterized in that the co-polyamino acid bearing carboxylate charges and hydrophobic radicals is selected from the co-polyamino acids according to formula VIIb wherein R₁ is a hydrophobic radical according to formula I, V or VI wherein r=0 or r=1 and GpR is according to formula II′.

In one embodiment, the composition is characterized in that the co-polyamino acid bearing carboxylate charges and hydrophobic radicals is selected from the co-polyamino acids according to formulas VIIb wherein R₂ is a hydrophobic radical according to formula I, V or VI wherein r=1 and GpR is according to formula II.

In one embodiment, the composition is characterized in that R₁ is a radical chosen from the group consisting of a C₂ to C₁₀ linear acyl group, a C₃ to C₁₀ branched acyl group, a benzyl, a terminal “amino acid” unit and a pyroglutamate.

In one embodiment, the composition is characterized in that R₁ is a radical chosen from the group consisting of linear acyl group C₂ to C₁₀ or a branched acyl group in C₃ to C₁₀.

In one embodiment, the composition is characterized in that the co-polyamino acid bearing carboxylate charges and hydrophobic radicals is selected from the co-polyamino acids according to formula VII, VIIa or VIIb wherein the group D is a —CH₂— (aspartic unit) group.

In one embodiment, the composition is characterized in that the co-polyamino acid bearing carboxylate charges and hydrophobic radicals is selected from the co-polyamino acids according to formula VII, VIIa or VIIb wherein the group D is a —CH₂—CH₂— (glutamic unit) group.

In one embodiment, the composition is characterized in that the ratio i between the number of hydrophobic radicals and the number of glutamic or aspartic units is comprised from 0.007 to 0.3.

In one embodiment, the composition is characterized in that the ratio i between the number of hydrophobic radicals and the number of glutamic or aspartic units is comprised from 0.01 to 0.3.

In one embodiment, the composition is characterized in that the ratio i between the number of hydrophobic radicals and the number of glutamic or aspartic units is comprised from 0.02 to 0.2.

In one embodiment, the composition is characterized in that the hydrophobic radical corresponds to formula VI and the ratio i between the number of hydrophobic radicals and the number of glutamic or aspartic units is comprised from 0.007 to 0.15.

In one embodiment, the composition is characterized in that the hydrophobic radical corresponds to formula VI and the ratio i between the number of hydrophobic radicals and the number of glutamic or aspartic units is comprised from 0.01 to 0.1.

In one embodiment, the composition is characterized in that the hydrophobic radical corresponds to formula VI and the ratio i between the number of hydrophobic radicals and the number of glutamic or aspartic units is comprised from 0.02 to 0.08.

In one embodiment, the composition is characterized in that the hydrophobic radical corresponds to formula VI wherein the radical Cx comprises between 9 and 10 carbon atoms and the ratio i between the number of hydrophobic radicals and the number of glutamic or aspartic units is comprised from 0.03 to 0.15.

In one embodiment, the composition is characterized in that the hydrophobic radical corresponds to formula VI wherein the radical Cx comprises between 11 and 12 carbon atoms and the ratio i between the number of hydrophobic radicals and the number of glutamic or aspartic units is comprised from 0.015 to 0.1.

In one embodiment, the composition is characterized in that the hydrophobic radical corresponds to formula VI wherein the radical Cx comprises between 11 and 12 carbon atoms and the ratio i between the number of hydrophobic radicals and the number of glutamic or aspartic units is comprised from 0.02 to 0.08.

In one embodiment, the composition is characterized in that the hydrophobic radical corresponds to formula VI wherein the radical Cx comprises between 13 and 15 carbon atoms and the ratio i between the number of hydrophobic radicals and the number of glutamic or aspartic units is comprised from 0.01 to 0.1.

In one embodiment, the composition is characterized in that the hydrophobic radical corresponds to formula VI wherein the radical Cx comprises between 13 and 15 carbon atoms and the ratio i between the number of hydrophobic radicals and the number of glutamic or aspartic units is comprised from 0.01 to 0.06.

In one embodiment, the composition is characterized in that the hydrophobic radical corresponds to formula V and the ratio i between the number of hydrophobic radicals and the number of glutamic or aspartic units is comprised from 0.007 to 0.3.

In one embodiment, the composition is characterized in that the hydrophobic radical corresponds to formula V and the ratio i between the number of hydrophobic radicals and the number of glutamic or aspartic units is comprised from 0.01 to 0.3.

In one embodiment, the composition is characterized in that the hydrophobic radical corresponds to formula V and the ratio i between the number of hydrophobic radicals and the number of glutamic or aspartic units is comprised from 0.015 to 0.2.

In one embodiment, the composition is characterized in that the hydrophobic radical corresponds to formula V wherein the radical Cx comprises from 11 to 14 carbon atoms and the ratio i between the number of hydrophobic radicals and the number of glutamic or aspartic units is comprised from 0.1 to 0.2.

In one embodiment, the composition is characterized in that the hydrophobic radical corresponds to formula V wherein the radical Cx comprises from 15 to 16 carbon atoms and the ratio i between the number of hydrophobic radicals and the number of glutamic or aspartic units is comprised from 0.04 to 0.15.

In one embodiment, the composition is characterized in that the hydrophobic radical corresponds to formula V wherein the radical Cx comprises from 17 to 18 carbon atoms and the ratio i between the number of hydrophobic radicals and the number of glutamic or aspartic units is comprised from 0.02 to 0.06.

In one embodiment, the composition is characterized in that the hydrophobic radical corresponds to formula V wherein the radical Cx comprises from 19 to 25 carbon atoms and the ratio i between the number of hydrophobic radicals and the number of glutamic or aspartic units is comprised from 0.01 to 0.06.

In one embodiment, the composition is characterized in that the hydrophobic radical corresponds to formula V wherein the radical Cx comprises from 19 to 25 carbon atoms and the ratio i between the number of hydrophobic radicals and the number of glutamic or aspartic units is comprised from 0.01 to 0.05.

In one embodiment, the composition according to the invention is characterized in that n+m is comprised from 10 to 250.

In one embodiment, the composition according to the invention is characterized in that n+m is comprised from 10 to 200.

In one embodiment, the composition according to the invention is characterized in that n+m is comprised from 15 to 150.

In one embodiment, the composition according to the invention is characterized in that n+m is comprised from 15 to 100.

In one embodiment, the composition according to the invention is characterized in that n+m is comprised from 15 to 80.

In one embodiment, the composition according to the invention is characterized in that n+m is comprised from 15 to 65.

In one embodiment, the composition according to the invention is characterized in that n+m is comprised from 20 to 60.

In one embodiment, the composition according to the invention is characterized in that n+m is comprised from 20 to 50.

In one embodiment, the composition according to the invention is characterized in that n+m is comprised from 20 to 40.

The invention also relates to said co-polyamino acids bearing carboxylate charges and hydrophobic radicals according to formula I and the precursors of said hydrophobic radicals.

In one embodiment, the concentration of co-polyamino acid bearing carboxylate charges and hydrophobic radicals is at most 60 mg/mL.

In one embodiment, the concentration of co-polyamino acid bearing carboxylate charges and hydrophobic radicals is at most 40 mg/mL.

In one embodiment, the concentration of co-polyamino acid bearing carboxylate charges and hydrophobic radicals is at most 20 mg/mL.

In one embodiment, the concentration of co-polyamino acid bearing carboxylate charges and hydrophobic radicals is at most 10 mg/mL.

In one embodiment, the concentration of co-polyamino acid bearing carboxylate charges and hydrophobic radicals is at most 5 mg/ml.

In one embodiment, the concentration of co-polyamino acid bearing carboxylate charges and hydrophobic radicals is at most 2.5 mg/ml.

The invention further relates to a method for preparing stable injectable compositions.

In one embodiment, the copolyamino acid is a sodium poly-L-glutamate modified at one of its extremity according to the formula represented hereinafter as described in example AB24.

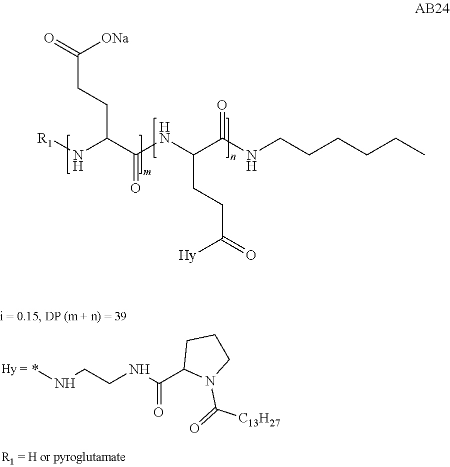

In one embodiment, copolyamino acid is a sodium poly-L-glutamate modified at one of its extremity according to the formula represented hereinafter as described in example AB22.

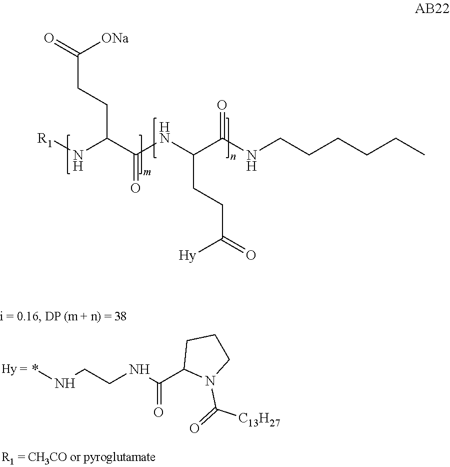

In one embodiment, copolyamino acid is a sodium poly-L-glutamate modified at one of its extremity according to the formula represented hereinafter as described in example AB35.

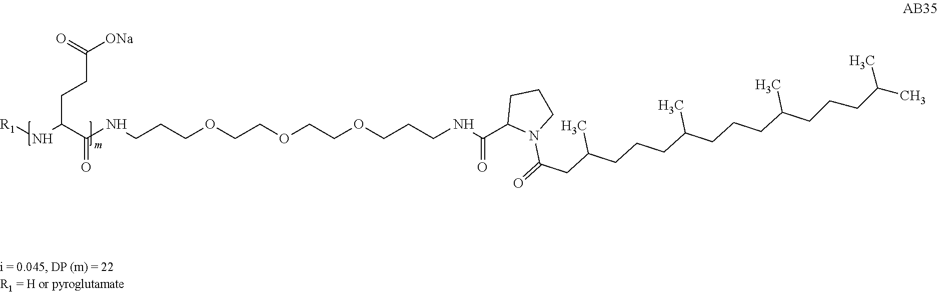

In one embodiment the co-polyamino acid is a sodium poly-L-glutamate modified at one of its extremity according to the formula represented hereinafter, described in examples BB15, BB17, BB18 and BB19.

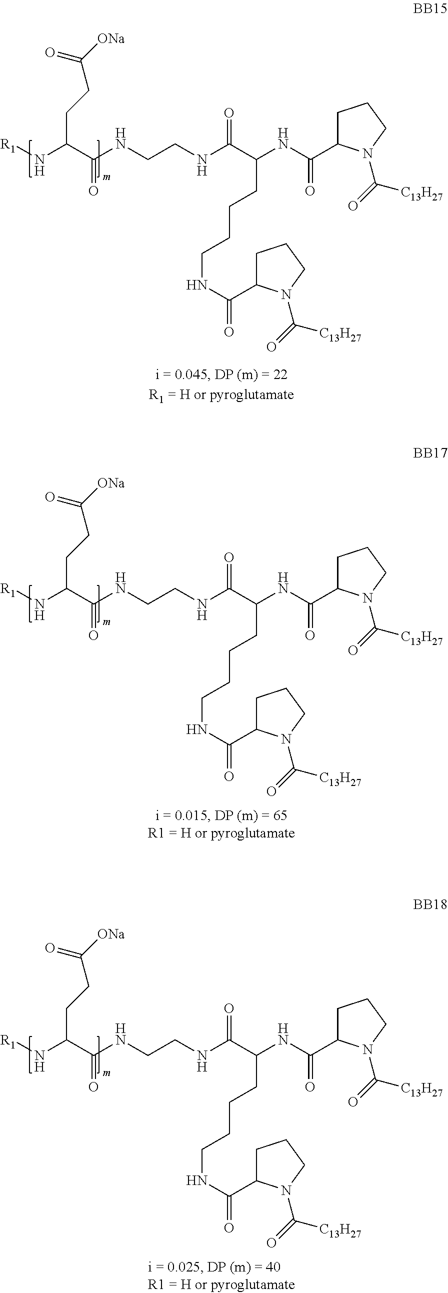

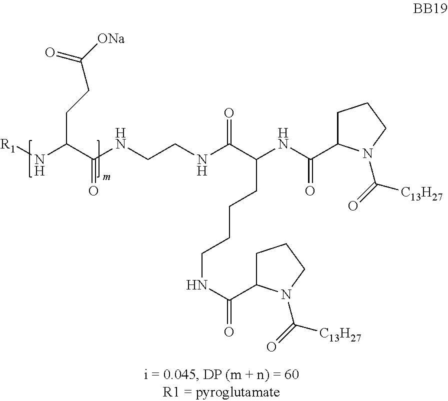

In one embodiment, the composition according to the invention is characterized in that the co-polyamino acid is derived from a polyamino acid obtained by polymerization.

In one embodiment, the composition according to the invention is characterized in that co-polyamino acid is derived from a polyamino acid obtained by ring opening polymerization of a glutamic acid N-carboxyanhydride derivative or a N-carboxyanhydride derivative of aspartic acid.

In one embodiment, the composition according to the invention is characterized in that co-polyamino acid is derived from a polyamino acid obtained by polymerization of a glutamic acid N-carboxyanhydride derivative or a N-derivative. Aspartic acid carboxyanhydride as described in Deming, T. J., Adv. Polym. Sci. 2006, 202, 1-18.

In one embodiment, the composition according to the invention is characterized in that co-polyamino acid is derived from a polyamino acid obtained by polymerization of a glutamic acid N-carboxyanhydride derivative.

In one embodiment, the composition according to the invention is characterized in that co-polyamino acid is derived from a polyamino acid obtained by polymerization of a glutamic acid N-carboxyanhydride derivative chosen from the group consisting of N-carboxyanhydride methyl polyglutamate (GluOMe-NCA), benzyl N-carboxyanhydride polyglutamate (GluOBzl-NCA) and t-butyl N-carboxyanhydride polyglutamate (GluOtBu-NCA).

In one embodiment, the glutamic acid N-carboxyanhydride derivative is methyl N-carboxyanhydride poly-L-glutamate (L-GluOMe-NCA).

In one embodiment, the glutamic acid N-carboxyanhydride derivative is benzyl N-carboxyanhydride poly-L-glutamate (L-GluOBzl-NCA).

In one embodiment, the composition according to the invention is characterized in that the co-polyamino acid is derived from a polyamino acid obtained by polymerization of a glutamic acid N-carboxy anhydride derivative or of a aspartic acid N-carboxy anhydride derivative using an organometallic complex of a transition metal as initiator as described in the publication Deming, T. J., Nature 1997, 390, 386-389.

In one embodiment, the composition according to the invention is characterized in that co-polyamino acid is derived from a polyamino acid obtained by polymerization of a glutamic acid N-carboxyanhydride derivative or an aspartic acid N-carboxyanhydride derivative using ammonia or a primary amine as initiator as described in FR 2,801,226 and references cited therein.

In one embodiment, the composition according to the invention is characterized in that the co-polyamino acid is derived from a polyamino acid obtained by polymerization of a glutamic acid N-carboxyanhydride derivative or an aspartic acid N-carboxyanhydride derivative using hexamethyldisilazane as initiator as described in Lu H.; et al., J. Am. Chem. Soc. 2007, 129, 14114-14115 or a silylated amine as described in the publication Lu H.; et al., J. Am. Chem. Soc. 2008, 130, 12562-12563.

In one embodiment, the composition according to the invention is characterized in that the process for synthesizing the polyamino acid obtained by polymerization of a glutamic acid N-carboxyanhydride derivative or an aspartic acid N-carboxyanhydride derivative acid from which the co-polyamino acid is derived, comprises a esther function hydrolysis step.

In one embodiment, this ester hydrolysis step may consist of hydrolysis in an acidic medium or hydrolysis in a basic medium or may be carried out by hydrogenation.

In one embodiment, this ester group hydrolysis step is a hydrolysis in an acidic medium.

In one embodiment, this ester group hydrolysis step is carried out by hydrogenation.

In one embodiment, the composition according to the invention is characterized in that co-polyamino acid is derived from a polyamino acid obtained by depolymerization of a polyamino acid of higher molecular weight.

In one embodiment, the composition according to the invention is characterized in that co-polyamino acid is derived from a polyamino acid obtained by enzymatic depolymerization of a polyamino acid of higher molecular weight.

In one embodiment, the composition according to the invention is characterized in that co-polyamino acid is derived from a polyamino acid obtained by chemical depolymerization of a polyamino acid of higher molecular weight.

In one embodiment, the composition according to the invention is characterized in that co-polyamino acid is derived from a polyamino acid obtained by enzymatic and chemical depolymerization of a polyamino acid of higher molecular weight.

In one embodiment, the composition according to the invention is characterized in that co-polyamino acid is derived from a polyamino acid obtained by depolymerization of a polyamino acid of higher molecular weight chosen from the group consisting of sodium polyglutamate and sodium polyaspartate.

In one embodiment, the composition according to the invention is characterized in that co-polyamino acid is derived from a polyamino acid obtained by depolymerization of a sodium polyglutamate of higher molecular weight.

In one embodiment, the composition according to the invention is characterized in that co-polyamino acid is derived from a polyamino acid obtained by depolymerization of a sodium polyaspartate of higher molecular weight.

In one embodiment, the composition according to the invention is characterized in that the co-polyamino acid is obtained by grafting a hydrophobic group onto an acid poly-L-glutamic acid or poly-L-aspartic acid using amide bond-forming methods well known to those skilled in the art.

In one embodiment, the composition according to the invention is characterized in that co-polyamino acid is obtained by grafting a hydrophobic group onto a poly-L-glutamic acid or poly-L-aspartic acid using amide bond formation processes used for peptide synthesis.

In one embodiment, the composition according to the invention is characterized in that the co-polyamino acid is obtained by grafting a hydrophobic group onto a poly-L-glutamic acid or poly-L-aspartic acid as described in patent FR 2,840,614.

The invention also relates to the co-polyamino acid bearing carboxylate charges and hydrophobic radicals Hy, said co-polyamino acid being constituted by glutamic or aspartic units and said hydrophobic radicals Hy selected from the radicals according to formula I as defined below:

*GpRᵣGpAₐGpC)ₚ Formula I

wherein
GpR is a radical according to formulas II, II′ or II″:

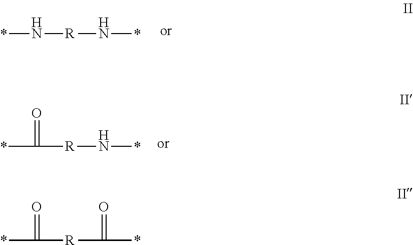

GpA is a radical according to formulas m or III′:

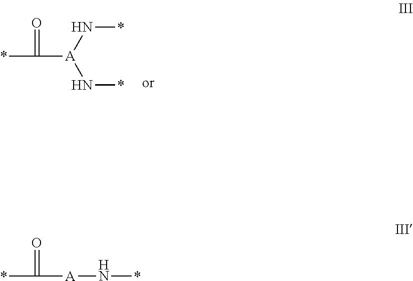

GpC is a radical according to formula IV:

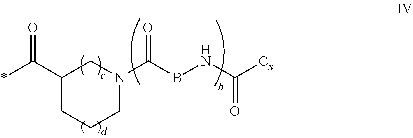

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula II′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      through a covalent bond between a nitrogen atom of the hydrophobic radial and a carbonyl of the copolyamino acid, thus forming an amide function originating from the reaction of an amine function of the precursor of the hydrophobic radical and an acid function borne by the precursor of the co-polyamino acid or
      through a covalent bond between a carbonyl of the hydrophobic radical and a nitrogen atom in N-terminal position of the co-polyamino acid, thus forming an amide function originating from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms; more precisely, R is a radical chosen from the group consisting of:
   a linear or branched divalent alkyl radical, comprising from 2 to 12 carbon atoms if GpR is a radical according to formula II or from 1 to 11 carbon atoms if GpR is a radical according to formula II′ or II″;
   a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
   an unsubstituted ether or polyether radical comprising from 4 to 14 carbon atoms and from 1 to 5 oxygen atoms;
A is a linear or branched alkyl radical comprising from 1 to 8 carbon atoms and optionally substituted by a radical resulting from a saturated, unsaturated or aromatic ring;
B is a radical chosen from the group consisting of an unsubstituted ether or polyether radical comprising from 4 to 14 carbon atoms and from 1 to 5 oxygen atoms or a linear or branched alkyl radical, optionally comprising an aromatic ring, comprising 1 to 9 carbon atoms;
B is a linear or branched alkyl radical, optionally comprising an aromatic ring, comprising from 1 to 9 carbon atoms;
Cₓ is a linear or branched monovalent alkyl radical, optionally comprising a cyclic part, wherein x indicates the number of carbon atoms and:
   if p is equal to 1, x is comprised from 9 to 25 (9≤x≤25):
   if p is equal to 2, x is comprised from 9 to 15 (9≤x≤15),
the ratio i between the number of hydrophobic radicals and the number of glutamic or aspartic units being from 0<i≤0.5;
when several hydrophobic radicals are carried by a co-polyamino acid they are therefore, identical or different;
the degree of polymerization DP of glutamic or aspartic units is comprised from 5 to 250; the free acid functions being in the form of an alkaline cation salt chosen from the group consisting of Na⁺ and K⁺

The invention also relates to the co-polyamino acid bearing carboxylate charges and hydrophobic radicals Hy, said co-polyamino acid being constituted by glutamic or aspartic units and said hydrophobic radicals Hy selected from the radicals according to formula I as defined below:

*GpRᵣGpAₐGpC)ₚ Formula I

wherein
GpR is a radical according to formulas II, II′ or II″:

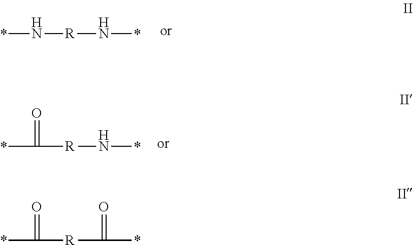

GpA is a radical according to formulas III or III′:

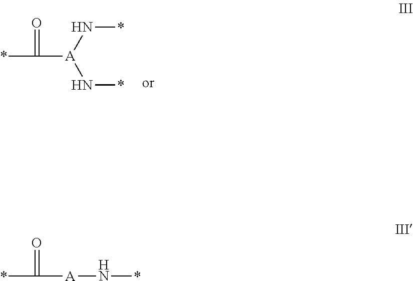

GpC is a radical according to formula IV:

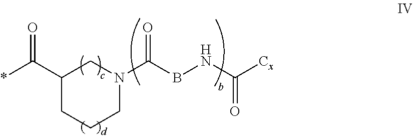

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      through a covalent bond between a nitrogen atom of the hydrophobic radial and a carbonyl of the copolyamino acid, thus forming an amide function originating from the reaction of an amine function of the precursor of the hydrophobic radical and an acid function borne by the precursor of the co-polyamino acid or
      through a covalent bond between a carbonyl of the hydrophobic radical and a nitrogen atom in N-terminal position of the co-polyamino acid, thus forming an amide function originating from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms; more precisely, R is a radical chosen from the group consisting of:
   a linear or branched divalent alkyl radical, comprising from 2 to 12 carbon atoms if GpR is a radical according to formula II or from 1 to 11 carbon atoms if GpR is a radical according to formula II′ or II″;
   a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
   an unsubstituted ether or polyether radical comprising from 4 to 14 carbon atoms and from 1 to 5 oxygen atoms;
A is a radical chosen from the group consisting of an unsubstituted ether or polyether radical comprising from 4 to 14 carbon atoms and from 1 to 5 oxygen atoms or a linear or branched alkyl radical comprising from 1 to 8 carbon atoms and optionally substituted by a radical derived from a saturated, unsaturated or aromatic ring;
B is a linear or branched alkyl radical, optionally comprising an aromatic ring, comprising from 1 to 9 carbon atoms;
Cₓ is a linear or branched monovalent alkyl radical, optionally comprising a cyclic part, wherein x indicates the number of carbon atoms and:
   if p is equal to 1, x is comprised from 9 to 25 (9≤x≤25):
   if p is equal to 2, x is comprised from 9 to 15 (9≤x≤15),
the ratio i between the number of hydrophobic radicals and the number of glutamic or aspartic units being from 0<i≤0.5;
when several hydrophobic radicals are carried by a co-polyamino acid they are therefore, identical or different;
the degree of polymerization DP of glutamic or aspartic units is comprised from 5 to 250;
   the free acid functions being in the form of an alkaline cation salt chosen from the group consisting of Na⁺ and K⁺

The invention also relates to the precursor Hy′ of the hydrophobic radical Hy according to formula I as defined below:

*GpRᵣGpAₐGpC)ₚ Formula I

wherein
GpR is a radical according to formulas II, I′ or II″:

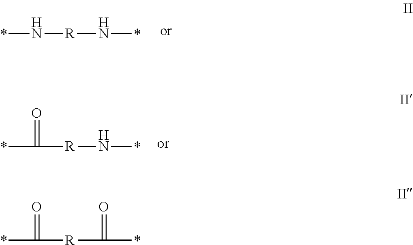

GpA is a radical according to formulas III or III′:

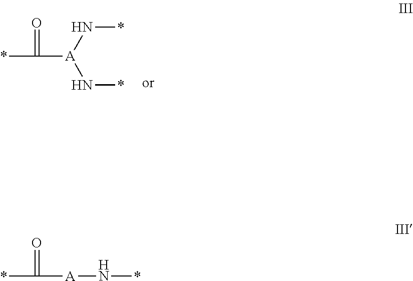

GpC is a radical according to formula IV:

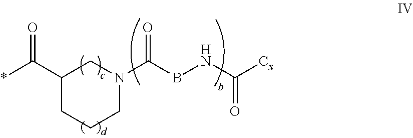

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      through a covalent bond between a nitrogen atom of the hydrophobic radial and a carbonyl of the copolyamino acid, thus forming an amide function originating from the reaction of an amine function of the precursor of the hydrophobic radical and an acid function borne by the precursor of the co-polyamino acid or
      through a covalent bond between a carbonyl of the hydrophobic radical and a nitrogen atom in N-terminal position of the co-polyamino acid, thus forming an amide function originating from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms; more precisely, R is a radical chosen from the group consisting of:
   a linear or branched divalent alkyl radical, comprising from 2 to 12 carbon atoms if GpR is a radical according to formula II or from 1 to 11 carbon atoms if GpR is a radical according to formula II′ or II″;
   a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
   an unsubstituted ether or polyether radical comprising from 4 to 14 carbon atoms and from 1 to 5 oxygen atoms;
A is a linear or branched alkyl radical comprising from 1 to 8 carbon atoms and optionally substituted by a radical resulting from a saturated, unsaturated or aromatic ring;
B is a radical chosen from the group consisting of an unsubstituted ether or polyether radical comprising from 4 to 14 carbon atoms and from 1 to 5 oxygen atoms or a linear or branched alkyl radical, optionally comprising an aromatic ring, comprising from 1 to 9 carbon atoms;
Cₓ is a linear or branched monovalent alkyl radical, optionally comprising a cyclic part, wherein x indicates the number of carbon atoms and:
   if p is equal to 1, x is comprised from 9 to 25 (9<x≤25):
   if p is equal to 2, x is comprised from 9 to 15 (9≤x≤15),
the ratio i between the number of hydrophobic radicals and the number of glutamic or aspartic units being between 0<i≤0.5;
when several hydrophobic radicals are carried by a co-polyamino acid they are therefore, identical or different;
the degree of polymerization DP of glutamic or aspartic units is comprised from 5 to 250;
the free acid functions being in the form of an alkaline cation salt chosen from the group consisting of Na⁺ and K⁺.

The invention also relates to the precursor Hy′ of the hydrophobic radical Hy according to formula I as defined below:

*GpRᵣGpAₐGpC)ₚ Formula I

wherein
GpR is a radical according to formulas II, r or II″:

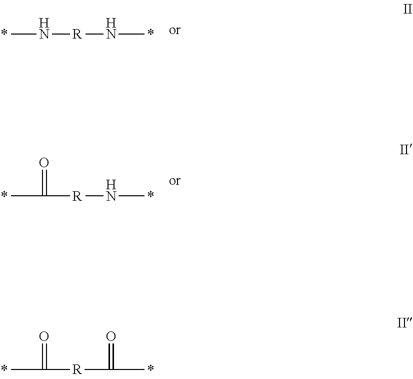

GpA is a radical according to formulas III or III′:

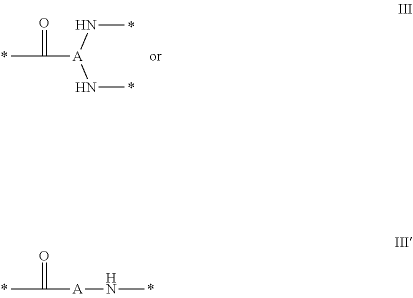

GpC is a radical according to formula IV:

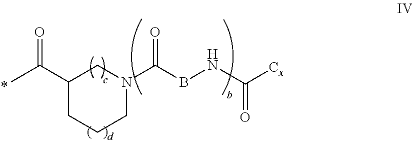

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      through a covalent bond between a nitrogen atom of the hydrophobic radial and a carbonyl of the copolyamino acid, thus forming an amide function originating from the reaction of an amine function of the precursor of the hydrophobic radical and an acid function borne by the precursor of the co-polyamino acid or
      through a covalent bond between a carbonyl of the hydrophobic radical and a nitrogen atom in N-terminal position of the co-polyamino acid, thus forming an amide function originating from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms; more precisely, R is a radical chosen from the group consisting of:
   a linear or branched divalent alkyl radical, comprising from 2 to 12 carbon atoms if GpR is a radical according to formula II or from 1 to 11 carbon atoms if GpR is a radical according to formula II′ or II″;
   a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
   an unsubstituted ether or polyether radical comprising from 4 to 14 carbon atoms and from 1 to 5 oxygen atoms;
A is a radical chosen from the group consisting of an unsubstituted ether or polyether radical comprising from 4 to 14 carbon atoms and from 1 to 5 oxygen atoms or a linear or branched alkyl radical comprising from 1 to 8 carbon atoms and optionally substituted by a radical derived from a saturated, unsaturated or aromatic ring;
B is a linear or branched alkyl radical, optionally comprising an aromatic ring, comprising from 1 to 9 carbon atoms;
Cₓ is a linear or branched monovalent alkyl radical, optionally comprising a cyclic part, wherein x indicates the number of carbon atoms and:
   if p is equal to 1, x is comprised from 9 to 25 (9≤x≤25):
   if p is equal to 2, x is comprised from 9 to 15 (9≤x≤15),
the ratio i between the number of hydrophobic radicals and the number of glutamic or aspartic units being from 0<i≤0.5;
when several hydrophobic radicals are carried by a co-polyamino acid they are therefore, identical or different;
the degree of polymerization DP of glutamic or aspartic units is comprised from 5 to 250;
the free acid functions being in the form of an alkaline cation salt chosen from the group consisting of Na⁺ and K⁺.

In one embodiment, the invention also relates to the precursors of said hydrophobic radicals according to formula V′ and VI′:

HGpRᵣGpAₐGpC formule V′

HGpRᵣGpAGpC)₂ formule VI′

wherein GpR, GpA, GpC, r and a have the definitions given above.

Insulin glargine or “islet amyloid polypeptide” (IAPP), is a 37 residue peptide hormone. It is co-secreted with insulin from pancreatic beta cells in the ratio of about 100/1. Glargine insulin plays a role in glycemic regulation by stopping the secretion of endogenous glucagon and slowing down gastric emptying and promoting satiety, thereby reducing postprandial glucose excursions in blood glucose levels.

IAPP is processed from a coding sequence of 89 residues. The amyloid polypeptide pro-islet (proIAPP, proamylin, proislet protein) is produced in pancreatic beta cells (beta cells) in the form of a pro-peptide of 67 amino acids, 7404 Daltons, and it undergoes post-translational modifications comprising protease cleavage to produce insulin glargine.

In the present application, insulin glargine as mentioned refers to the compounds described in U.S. Pat. Nos. 5,124,314 and 5,234,906.

The term “analogue”, when used in reference to a peptide or a protein, a peptide or a protein, wherein one or more constituent amino acid residues of the primary sequence have been substituted by other residues of amino acids and/or wherein one or more constituent amino acid residues have been deleted and/or wherein one or more constituent amino acid residues have been added. The percentage of homology allowed for the current definition of an analogue is 50%. In the case of insulin glargine, an analog may for example be derived from the primary amino acid sequence of insulin glargine by replacing one or more natural or non-natural or peptidomimetic amino acids.

The term “derivative”, is used in reference to a peptide or a protein, a peptide or protein or an analogue chemically modified with a substituent that is not present in the peptide or protein or analog reference, in other words, a peptide or protein that has been modified by covalent bonding to introduce non-amino acid substituents.

Hereinafter, the units used for insulins are those recommended by pharmacopoeias, whose mg/ml equivalences are provided in the table below in:

| Insulin | EP Pharmacopoeia 8.0 (2014) | US Pharmacopoeia - USP38 (2015) |
|---|---|---|
| Aspart | 1 U = 0.0350 mg of insulin aspart | 1 USP = 0.0350 mg of insulin aspart |
| Lispro | 1 U = 0.0347 mg of lispro insulin | 1 USP = 0.0347 mg of lispro insulin |
| Human | 1 IU = 0.0347 mg of human insulin | 1 USP = 0.0347 mg of human insulin |
| Glargine | 1 U = 0.0364 mg of insulin glargine | 1 USP = 0.0364 mg of insulin glargine |
| Porcine | 1 IU = 0.0345 mg of porcine insulin | 1 USP = 0.0345 mg of porcine insulin |
| Bovine | 1 IU = 0.0342 mg of bovine insulin | 1 USP = 0.0342 mg of bovine insulin |

Basal insulin, whose isoelectric point is comprised from 5.8 to 8.5, refers to an insulin that is insoluble at pH 7 and whose duration of action is comprised from 8 to 24 hours or higher in standard diabetes models.

These basal insulins whose isoelectric point is comprised from 5.8 to 8.5 are recombinant insulins which primary structure has been modified mainly by the introduction of basic amino acids, such as Arginine or Lysine. They are described for example, in the following patents, patent applications or publications WO 2003/053339, WO 2004/096854, U.S. Pat. Nos. 5,656,722 and 6,100,376 whose content is incorporated by reference.

In one embodiment, the basal insulin whose isoelectric point is comprised from 5.8 to 8.5 is insulin glargine. Insulin glargine is marketed under the brand name Lantus® (100 U/ml) or Toujeo® (300 U/ml) by SANOFI.

In one embodiment, basal insulin whose isoelectric point is comprised from 5.8 to 8.5 is a biosimilar insulin glargine.

A biosimilar insulin glargine is in the process of being marketing under the brand name Abasaglar® or Basaglar® by ELI LILLY.

In one embodiment, the compositions according to the invention contain between 40 and 500 U/mL of basal insulin whose isoelectric point is comprised from 5.8 to 8.5.

In one embodiment, the compositions according to the invention comprise 40 U/mL of basal insulin whose isoelectric point is comprised from 5.8 to 8.5.

In one embodiment, the compositions according to the invention comprise 75 U/mL of basal insulin whose isoelectric point is comprised from 5.8 to 8.5.

In one embodiment, the compositions according to the invention contain 100 U/mL (i.e. approximately 3.6 mg/mL) of basal insulin whose isoelectric point is comprised from 5.8 to 8.5.

In one embodiment, the compositions according to the invention comprise 150 U/mL of basal insulin whose isoelectric point is comprised from 5.8 to 8.5.

In one embodiment, the compositions according to the invention comprise 200 U/mL of basal insulin whose isoelectric point is comprised from 5.8 to 8.5.

In one embodiment, the compositions according to the invention comprise 225 U/mL of basal insulin whose isoelectric point is comprised from 5.8 to 8.5.

In one embodiment, the compositions according to the invention comprise 250 U/mL of basal insulin whose isoelectric point is comprised from 5.8 to 8.5.

In one embodiment, the compositions according to the invention comprise 300 U/mL of basal insulin whose isoelectric point is comprised from 5.8 to 8.5.

In one embodiment, the compositions according to the invention comprise 400 U/mL of basal insulin whose isoelectric point is comprised from 5.8 to 8.5.

In one embodiment, the compositions according to the invention comprise 500 U/mL of basal insulin whose isoelectric point is comprised from 5.8 to 8.5.

In one embodiment the mass ratio between the basal insulin whose isoelectric point is comprised from 5.8 to 8.5 and the co-polyamino acid, i.e. co-polyamino acid/basal insulin, is comprised from 0.2 to 8.

In one embodiment the mass ratio is comprised from 0.2 to 6.

In one embodiment the mass ratio is comprised from 0.2 to 5.

In one embodiment the mass ratio is comprised from 0.2 to 4.

In one embodiment the mass ratio is comprised from 0.2 to 3.

In one embodiment the mass ratio is comprised from 0.2 to 2.

In one embodiment the mass ratio is comprised from 0.2 to 1.

In one embodiment the compositions according to the invention comprise a prandial insulin. Prandial insulins are soluble at pH 7.

prandial insulin refers to fast or “regular” insulin.

Prandial insulins termed ‘rapid’ are insulins that must satisfy the needs caused by the ingestion of proteins and carbohydrates during a meal, they must react in less than 30 minutes.

In one embodiment, “regular” prandial insulin is human insulin.

In one embodiment, prandial insulin is a recombinant human insulin as described in the European Pharmacopoeia and the American Pharmacopoeia.

Human insulin is for example marketed under the brand names Humulin® (ELI LILLY) and Novolin® (NOVO NORDISK).

Fast-acting prandial insulins are insulins that are obtained by recombination and whose primary structure has been modified to reduce their reaction time.

In one embodiment, the said (very fast-acting) prandial insulins are chosen from the group comprising insulin lispro (Humalog®), insulin glulisine (Apidra®) and insulin aspart (NovoLog®).

In one embodiment, prandial insulin is insulin lispro.

In one embodiment, prandial insulin is insulin glulisine.

In one embodiment, prandial insulin is insulin aspart.

In one embodiment, the compositions according to the invention comprise in total between 60 and 800 U/mL of insulin with a combination of prandial insulin and basal insulin whose isoelectric point is comprised from 5.8 to 8.5.

In one embodiment, the compositions according to the invention comprise in total between 100 and 500 U/mL of insulin with a combination of prandial insulin and basal insulin whose isoelectric point is comprised from 5.8 to 8.5.

In one embodiment, the compositions according to the invention comprise a total of 800 U/mL of insulin with a combination of prandial insulin and basal insulin whose isoelectric point is comprised from 5.8 to 8.5.

In one embodiment, the compositions according to the invention comprise a total of 700 U/mL of insulin with a combination of prandial insulin and basal insulin whose isoelectric point is comprised from 5.8 to 8.5.

In one embodiment, the compositions according to the invention comprise a total of 600 U/mL of insulin with a combination of prandial insulin and basal insulin whose isoelectric point is comprised from 5.8 to 8.5.

In one embodiment, the compositions according to the invention comprise a total of 500 U/mL of insulin with a combination of prandial insulin and basal insulin whose isoelectric point is comprised from 5.8 to 8.5.

In one embodiment, the compositions according to the invention comprise a total of 400 U/mL of insulin with a combination of prandial insulin and basal insulin whose isoelectric point is comprised from 5.8 to 8.5.

In one embodiment, the compositions according to the invention comprise a total of 300 U/mL of insulin with a combination of prandial insulin and basal insulin whose isoelectric point is comprised from 5.8 to 8.5.

In one embodiment, the compositions according to the invention comprise a total of 266 U/mL of insulin with a combination of prandial insulin and basal insulin whose isoelectric point is comprised from 5.8 to 8.5.

In one embodiment, the compositions according to the invention comprise a total of 200 U/mL of insulin with a combination of prandial insulin and basal insulin whose isoelectric point is comprised from 5.8 to 8.5.

In one embodiment, the compositions according to the invention comprise a total of 100 U/mL of insulin with a combination of prandial insulin and basal insulin whose isoelectric point is comprised from 5.8 to 8.5.

The proportions between basal insulin whose isoelectric point is comprised from 5.8 to 8.5 and prandial insulin are, for example, in percentages of 25/75, 30/70, 40/60, 50/50, 60/40, 63/37, 70/30, 75/25, 80/20, 83/17, 90/10 for formulations as described above comprising from 60 to 800 U/mL. However, any other proportion can be achieved.

In one embodiment, basal insulin whose isoelectric point is comprised from 5.8 to 8.5 and prandial insulin are respectively present in the following concentrations (in U/ml) 75/25, 150/50, 200/66 or 300/100.

In one embodiment, the basal insulin whose isoelectric point is comprised from 5.8 to 8.5 and the prandial insulin are respectively present in the following concentrations (in U/ml) 75/25.

In one embodiment, the basal insulin whose isoelectric point is comprised from 5.8 to 8.5 and the prandial insulin are respectively present in the following concentrations (in U/ml) 150/50.

In one embodiment the compositions according to the invention comprise a gastrointestinal hormone.

“Gastrointestinal hormones”, refer to hormones chosen from the group consisting of GLP-1 RA (Glucagon like peptide-1 receptor agonist) and GIP (Glucose-dependent insulinotropic peptide), oxyntomodulin (a derivative of proglucagon), peptide YY, amylin, cholecystokinin, pancreatic peptide (PP), ghrelin and enterostatin, their analogs or derivatives and/or their pharmaceutically acceptable salts.

In one embodiment, gastrointestinal hormones are analogues or derivatives of GLP-1 RA chosen from the group consisting of exenatide or Byetta® (ASTRA-ZENECA), liraglutide or Victoza® (NOVO NORDISK), Lixisenatide or Lyxumia® (SANOFI), albiglutide or Tanzeum® (GSK) or dulaglutide or Trulicity® (ELI LILLY & CO), their analogues or derivatives and their pharmaceutically acceptable salts.

In one embodiment, the gastrointestinal hormone is pramlintide or Symlin® (ASTRA-ZENECA).

In one embodiment, the gastrointestinal hormone is exenatide or Byetta®, its analogs or derivatives and their pharmaceutically acceptable salts.

In one embodiment, the gastrointestinal hormone is liraglutide or Victoza®, its analogs or derivatives and their pharmaceutically acceptable salts.

In one embodiment, the gastrointestinal hormone is lixisenatide or Lyxumia®, its analogs or derivatives and their pharmaceutically acceptable salts.

In one embodiment, the gastrointestinal hormone is albiglutide or Tanzeum®, its analogs or derivatives and their pharmaceutically acceptable salts.

In one embodiment, the gastrointestinal hormone is dulaglutide or Trulicity®, its analogs or derivatives and their pharmaceutically acceptable salts.

In one embodiment, the gastrointestinal hormone is pramlintide or Symlin®, its analogs or derivatives and their pharmaceutically acceptable salts.

The term “analogue”, is used in reference to a peptide or a protein, a peptide or a protein, wherein one or more constituent amino acid residues have been substituted by other amino acid residues and/or wherein one or more constituent amino acid residues have been deleted and/or wherein one or more constituent amino acid residues have been added. The percentage of homology allowed for the current definition of an analogue is 50%.

The term “derivative”, is used in reference to a peptide or a protein, a peptide or protein or an analogue chemically modified with a substituent that is not present in the peptide or protein or analog reference, in other words, a peptide or protein that has been modified by covalent bonding to introduce substituents.

In one embodiment, the substituent is chosen from the group consisting of fatty chains.

In one embodiment, the concentration of gastrointestinal hormone ranges from 0.01 to 100 mg/mL.

In one embodiment, the concentration of exenatide, its analogs or derivatives and their pharmaceutically acceptable salts falls within a range of 0.04 to 0.5 mg/mL.

In one embodiment, the concentration of liraglutide, its analogs or derivatives and their pharmaceutically acceptable salts falls within a range of 1 to 10 mg/mL.

In one embodiment, the concentration of lixisenatide, its analogs or derivatives and their pharmaceutically acceptable salts falls within a range of 0.01 to 1 mg/mL.

In one embodiment, the concentration of albiglutide, its analogs or derivatives and their pharmaceutically acceptable salts is comprised from 5 to 100 mg/mL.

In one embodiment, the concentration of dulaglutide, its analogs or derivatives and their pharmaceutically acceptable salts is comprised from 0.1 to 10 mg/mL.

In one embodiment, the concentration of pramlintide, its analogs or derivatives and their pharmaceutically acceptable salts is comprised from 0.1 to 5 mg/mL.

In one embodiment the compositions according to the invention are produced by mixing commercial solutions of basal insulin whose isoclectric point is comprised from 5.8 to 8.5 and commercial solutions of GLP-1 RA, GLP-1 RA analog or derivative in volume ratios in a range of 10/90 to 90/10.

In one embodiment the composition according to the invention comprises a daily dose of basal insulin and a daily dose of gastrointestinal hormone.

In one embodiment, the compositions according to the invention comprise between 40 U/mL and 500 U/mL of basal insulin whose isoelectric point is comprised from 5.8 to 8.5 and between 0.05 and 0.5 mg/mL of exenatide.

In one embodiment, the compositions according to the invention comprise between 40 U/mL and 500 U/mL of basal insulin whose isoelectric point is comprised from 5.8 to 8.5 and between 1 and 10 mg/mL of liraglutide.

In one embodiment, the compositions according to the invention comprise between 40 U/mL and 500 U/mL of basal insulin whose isoelectric point is comprised from 5.8 to 8.5 and between 0.01 and 1 mg/mL of lixisenatide.

In one embodiment, the compositions according to the invention comprise between 40 U/mL and 500 U/mL of basal insulin whose isoelectric point is comprised from 5.8 to 8.5 and between 5 and 100 mg/mL of albiglutide.

In one embodiment, the compositions according to the invention comprise between 40 U/mL and 500 U/mL of basal insulin whose isoelectric point is comprised from 5.8 to 8.5 and between 0.1 and 10 mg/mL of dulaglutide.

In one embodiment, the compositions according to the invention comprise 500 U/mL of basal insulin whose isoelectric point is comprised from 5.8 to 8.5 and 0.04 to 0.5 mg/mL of exenatide.

In one embodiment, the compositions according to the invention comprise 500 U/mL of basal insulin whose isoelectric point is comprised from 5.8 to 8.5 and from 1 to 10 mg/mL of liraglutide.

In one embodiment, the compositions according to the invention comprise 500 U/mL of basal insulin whose isoelectric point is comprised from 5.8 to 8.5 and from 0.01 to 1 mg/mL of lixisenatide.

In one embodiment, the compositions according to the invention comprise 500 U/mL of basal insulin whose isoelectric point is comprised from 5.8 to 8.5 and 5 to 100 mg/mL of albiglutide.

In one embodiment, the compositions according to the invention comprise 500 U/mL of basal insulin whose isoelectric point is comprised from 5.8 to 8.5 and from 0.1 to 10 mg/mL of dulaglutide.

In one embodiment, the compositions according to the invention comprise 400 U/mL of basal insulin whose isoelectric point is comprised from 5.8 to 8.5 and 0.04 to 0.5 mg/mL of exenatide.

In one embodiment, the compositions according to the invention comprise 400 U/mL of basal insulin whose isoelectric point is comprised from 5.8 to 8.5 and from 1 to 10 mg/mL of liraglutide.

In one embodiment, the compositions according to the invention comprise 400 U/mL of basal insulin whose isoelectric point is comprised from 5.8 to 8.5 and from 0.01 to 1 mg/mL of lixisenatide.

In one embodiment, the compositions according to the invention comprise 400 U/mL of basal insulin whose isoelectric point is comprised from 5.8 to 8.5 and 5 to 100 mg/mL of albiglutide.

In one embodiment, the compositions according to the invention comprise 400 U/mL of basal insulin whose isoelectric point is comprised from 5.8 to 8.5 and from 0.1 to 10 mg/mL of dulaglutide.

In one embodiment, the compositions according to the invention comprise 300 U/mL of basal insulin whose isoelectric point is comprised from 5.8 to 8.5 and 0.04 to 0.5 mg/mL of exenatide.

In one embodiment, the compositions according to the invention comprise 300 U/mL of basal insulin whose isoelectric point is comprised from 5.8 to 8.5 and from 1 to 10 mg/mL of liraglutide.

In one embodiment, the compositions according to the invention comprise 300 U/mL of basal insulin whose isoelectric point is comprised from 5.8 to 8.5 and from 0.01 to 1 mg/mL of lixisenatide.

In one embodiment, the compositions according to the invention comprise 300 U/mL of basal insulin whose isoelectric point is comprised from 5.8 to 8.5 and 5 to 100 mg/mL of albiglutide.

In one embodiment, the compositions according to the invention comprise 300 U/mL of basal insulin whose isoelectric point is comprised from 5.8 to 8.5 and from 0.1 to 10 mg/mL of dulaglutide.

In one embodiment, the compositions according to the invention comprise 225 U/mL of basal insulin whose isoelectric point is comprised from 5.8 to 8.5 and 0.04 to 0.5 mg/mL of exenatide.

In one embodiment, the compositions according to the invention comprise 225 U/mL of basal insulin whose isoelectric point is comprised from 5.8 to 8.5 and from 1 to 10 mg/mL of liraglutide.

In one embodiment, the compositions according to the invention comprise 225 U/mL of basal insulin whose isoelectric point is comprised from 5.8 to 8.5 and from 0.01 to 1 mg/mL of lixisenatide.

In one embodiment, the compositions according to the invention comprise 225 U/mL of basal insulin whose isoelectric point is comprised from 5.8 to 8.5 and 5 to 100 mg/mL of albiglutide.

In one embodiment, the compositions according to the invention comprise 225 U/mL of basal insulin whose isoelectric point is comprised from 5.8 to 8.5 and from 0.1 to 10 mg/mL of dulaglutide.

In one embodiment, the compositions according to the invention comprise 200 U/mL of basal insulin whose isoelectric point is comprised from 5.8 to 8.5 and 0.04 to 0.5 mg/mL of exenatide.

In one embodiment, the compositions according to the invention comprise 200 U/mL of basal insulin whose isoelectric point is comprised from 5.8 to 8.5 and from 1 to 10 mg/mL of liraglutide.

In one embodiment, the compositions according to the invention comprise 200 U/mL of basal insulin whose isoelectric point is comprised from 5.8 to 8.5 and from 0.01 to 1 mg/mL of lixisenatide.

In one embodiment, the compositions according to the invention comprise 200 U/mL of basal insulin whose isoelectric point is comprised from 5.8 to 8.5 and 5 to 100 mg/mL of albiglutide.

In one embodiment, the compositions according to the invention comprise 200 U/mL of basal insulin whose isoelectric point is comprised from 5.8 to 8.5 and from 0.1 to 10 mg/mL of dulaglutide.

In one embodiment, the compositions according to the invention comprise 100 U/mL (i.e. about 3.6 mg/mL) of basal insulin whose isoelectric point is comprised from 5.8 to 8.5 and between 0.04 and 0.5 mg/mL of exenatide.

In one embodiment, the compositions according to the invention comprise 100 U/mL (i.e. about 3.6 mg/mL) of basal insulin whose isoelectric point is comprised from 5.8 to 8.5 and between 1 and 10 mg/mL of liraglutide.

In one embodiment, the compositions according to the invention comprise 100 U/mL (i.e. about 3.6 mg/mL) of basal insulin whose isoelectric point is comprised from 5.8 to 8.5 and between 0.01 and 1 mg/mL of lixisenatide.

In one embodiment, the compositions according to the invention comprise 100 U/mL of basal insulin whose isoelectric point is comprised from 5.8 to 8.5 and 5 to 100 mg/mL of albiglutide.

In one embodiment, the compositions according to the invention comprise 100 U/mL of basal insulin whose isoelectric point is comprised from 5.8 to 8.5 and from 0.1 to 10 mg/mL of dulaglutide.

In one embodiment, the compositions according to the invention comprise 40 U/mL of basal insulin whose isoclectric point is comprised from 5.8 to 8.5 and 0.04 to 0.5 mg/mL of exenatide.

In one embodiment, the compositions according to the invention comprise 40 U/mL of basal insulin whose isoelectric point is comprised from 5.8 to 8.5 and from 1 to 10 mg/mL of liraglutide.

In one embodiment, the compositions according to the invention comprise 40 U/mL of basal insulin whose isoelectric point is comprised from 5.8 to 8.5 and from 0.01 to 1 mg/mL of lixisenatide.

In one embodiment, the compositions according to the invention comprise 40 U/mL of basal insulin whose isoelectric point is comprised from 5.8 to 8.5 and 5 to 100 mg/mL of albiglutide.

In one embodiment, the compositions according to the invention comprise 40 U/mL of basal insulin whose isoelectric point is comprised from 5.8 to 8.5 and from 0.1 to 10 mg/mL of dulaglutide.

In one embodiment, the compositions according to the invention further comprise zinc salts at a concentration comprised between 0 and 5000 μM.

In one embodiment, the compositions according to the invention further comprise zinc salts at a concentration comprised between 0 and 4000 μM.

In one embodiment, the compositions according to the invention further comprise zinc salts at a concentration comprised between 0 and 3000 μM.

In one embodiment, the compositions according to the invention further comprise zinc salts at a concentration comprised between 0 and 2000 μM.

In one embodiment, the compositions according to the invention further comprise zinc salts at a concentration comprised between 0 and 1000 μM.

In one embodiment, the compositions according to the invention further comprise zinc salts at a concentration comprised between 50 and 600 μM.

In one embodiment, the compositions according to the invention further comprise zinc salts at a concentration comprised between 100 and 500 μM.

In one embodiment, the compositions according to the invention further comprise zinc salts at a concentration comprised between 200 and 500 μM.

In one embodiment, the compositions according to the invention further comprise buffers.

In one embodiment, the compositions according to the invention comprise buffers at concentrations comprised between 0 and 100 mM.

In one embodiment, the compositions according to the invention comprise buffers at concentrations comprised between 15 and 50 mM.

In one embodiment, the compositions of the invention comprise a buffer chosen from the group consisting of a phosphate buffer, Tris (trishydroxymethylaminomethane) and sodium citrate.

In one embodiment, the buffer is sodium phosphate.

In one embodiment, the buffer is Tris (trishydroxymethylaminomethane).

In one embodiment, the buffer is sodium citrate.

In one embodiment, the compositions according to the invention further comprise preservatives.

In one embodiment, the preservatives are chosen from the group consisting of m-cresol and phenol, alone or in a mixture.

In one embodiment, the concentration of preservatives is comprised from 10 to 50 mM.

In one embodiment, the concentration of preservatives is comprised from 10 to 40 mM.

In one embodiment, the compositions according to the invention further comprise a surfactant.

In one embodiment, the surfactant is chosen from the group consisting of propylene glycol and polysorbate.

The compositions according to the invention may further comprise additives such as tonicity agents.

In one embodiment, the tonicity agents are chosen from the group consisting of glycerine, sodium chloride, mannitol and glycine.

The compositions according to the invention may furthermore comprise all excipients conforming to pharmacopoeia and compatible with the insulins used at usage concentrations.

The invention also relates to a pharmaceutical formulation according to the invention, characterized in that it is obtained by drying and/or lyophilization.

In the case of local and systemic releases, the proposed modes of administration are intravenous, subcutaneous, intradermal or intramuscular.

Transdermal, oral, nasal, vaginal, ocular, oral, and pulmonary routes of administration are also envisaged.

In one embodiment, the composition according to the invention is characterized in that it is administered once a day.

In one embodiment the composition according to the invention is characterized in that it is administered at least twice a day.

In one embodiment the composition according to the invention is characterized in that it is administered twice a day.

In one embodiment, the composition according to the invention is characterized in that it further comprises prandial insulin.

In one embodiment, the composition according to the invention further comprising at least one prandial insulin is characterized in that it is administered once a day.

In one embodiment, the composition according to the invention further comprising at least one prandial insulin is characterized in that it is administered at least twice a day.

In one embodiment, the composition according to the invention further comprising at least one prandial insulin is characterized in that it is administered twice a day.

In one embodiment, the composition according to the invention is characterized in that it further comprises a gastrointestinal hormone.

In one embodiment, the composition according to the invention further comprising at least one gastrointestinal hormone is characterized in that it is administered once a day.

In one embodiment, the composition according to the invention further comprising at least one gastrointestinal hormone is characterized in that it is administered at least twice a day.

In one embodiment, the composition according to the invention further comprising at least one gastrointestinal hormone is characterized in that it is administered twice a day.

In one embodiment the composition according to the invention is characterized in that the gastrointestinal hormone is a GLP-1 RA.

In one embodiment, the composition according to the invention further comprising a GLP-1 RA is characterized in that it is administered once a day.

In one embodiment, the composition according to the invention additionally comprising at least one GLP-1 RA is characterized in that it is administered at least twice a day.

In one embodiment, the composition according to the invention further comprising at least one GLP-1 RA is characterized in that it is administered twice a day.

The invention also relates to single dose formulations at a pH between 6.0 and 8.0 comprising a basal insulin whose isoelectric point is comprised from 5.8 to 8.5 and a prandial insulin.

The invention also relates to single-dose formulations at a pH comprised between 6.0 and 8.0 comprising a basal insulin whose isoelectric point is comprised from 5.8 to 8.5 and a gastrointestinal hormone, as defined above.

The invention also relates to single dose formulations with a pH between 6.0 and 8.0 comprising a basal insulin whose isoelectric point is comprised from 5.8 to 8.5, a prandial insulin and a gastrointestinal hormone as defined above.

The invention also relates to single dose formulations with a pH between 6.6 and 7.8 comprising a basal insulin whose isoelectric point is comprised from 5.8 to 8.5 and a prandial insulin.

The invention also relates to single-dose formulations at a pH comprised between 6.6 and 7.8 comprising a basal insulin whose isoelectric point is comprised from 5.8 to 8.5 and a gastrointestinal hormone, as defined above.

The invention also relates to single dose formulations with a pH between 6.6 and 7.8 comprising a basal insulin whose isoelectric point is comprised from 5.8 to 8.5, a prandial insulin and a gastrointestinal hormone as defined above.

The invention also relates to single dose formulations with a pH between 6.6 and 7.6 comprising a basal insulin whose isoelectric point is comprised from 5.8 to 8.5 and a prandial insulin.

The invention also relates to single-dose formulations at a pH comprised between 6.6 and 7.6 comprising a basal insulin whose isoelectric point is comprised from 5.8 to 8.5 and a gastrointestinal hormone, as defined above.

The invention also relates to single dose formulations with a pH between 6.6 and 7.6 comprising a basal insulin whose isoelectric point is comprised from 5.8 to 8.5, a prandial insulin and a gastrointestinal hormone as defined above.

In one embodiment, the single-dose formulations further comprise a co-polyamino acid as defined above.

In one embodiment, the formulations are in the form of an injectable solution.

In one embodiment, the basal insulin whose isoelectric point is comprised from 5.8 to 8.5 is insulin glargine.

In one embodiment, the prandial insulin is human insulin.

In one embodiment, the insulin is a recombinant human insulin as described in the European Pharmacopoeia and the American Pharmacopoeia.

In one embodiment the prandial insulin is chosen from the group consisting of insulin lispro (Humalog®), insulin glulisine (Apidra®) and insulin aspart (NovoLog®).

In one embodiment, prandial insulin is insulin lispro.

In one embodiment, prandial insulin is insulin glulisine.

In one embodiment, prandial insulin is insulin aspart.

In one embodiment the GLP-1 RA, analog or derivative of GLP-1 RA is chosen from the group consisting of exenatide (Byetta®), liraglutide (Victoza®), lixisenatide (Lyxumia®), albiglutide (Tanzeum®), dulaglutide (Trulicity®) or one of their derivatives.

In one embodiment, the gastrointestinal hormone is exenatide.

In one embodiment, the gastrointestinal hormone is liraglutide.

In one embodiment, the gastrointestinal hormone is lixisenatide.

In one embodiment, the gastrointestinal hormone is albiglutide.

In one embodiment, the gastrointestinal hormone is dulaglutide.

The solubilization at pH between 6.0 and 8.0 of the basal insulins whose isoelectric point is comprised from 5.8 to 8.5, by the co-polyamino acids bearing carboxylate charges and at least one hydrophobic radical according to the invention, may easily be observed and monitored with the naked eye, thanks to a change in the appearance of the solution.

The solubilization at pH between 6.6 and 7.8 of the basal insulins whose isoelectric point is comprised from 5.8 to 8.5, by the co-polyamino acids bearing carboxylate charges and at least one hydrophobic radical according to the invention, may easily be observed and monitored with the naked eye, thanks to a change in the appearance of the solution.

In addition, and just as importantly, the applicant has been able to verify that a basal insulin whose isoelectric point is comprised from 5.8 to 8.5, solubilized at a pH comprised between 6.0 and 8.0 in the presence of a co-polyamino acid bearing carboxylate charges and at least one hydrophobic radical according to the invention, preserves its slow-acting insulin action whether alone or in combination with a prandial insulin or a gastrointestinal hormone.

The applicant has also been able to verify that a prandial insulin, mixed at a pH comprised between 6.0 and 8.0 in the presence of a co-polyamino acid bearing carboxylate charges and at least one hydrophobic radical according to the invention and a basal insulin whose isoelectric point is comprised from 5.8 to 8.5, preserves its rapid-acting insulin action.

The preparation of a composition according to the invention has the advantage of being able to be achieved by simple mixing of an aqueous solution of basal insulin whose isoelectric point is comprised from 5.8 to 8.5 and a co-polyamino acid bearing carboxylate charges and at least one hydrophobic radical according to the invention, in aqueous solution or in freeze-dried form. If necessary, the pH of the preparation is adjusted to a pH comprised between 6 and 8.

The preparation of a composition according to the invention has the advantage of being able to be achieved by simple mixing of an aqueous solution of basal insulin whose isoelectric point is comprised from 5.8 to 8.5, a prandial insulin and a co-polyamino acid bearing carboxylate charges and at least one hydrophobic radical according to the invention, as an aqueous solution or in freeze-dried form. If necessary, the pH of the preparation is adjusted to a pH comprised between 6 and 8.

The preparation of a composition according to the invention has the advantage of being able to be achieved by simple mixing of an aqueous solution of basal insulin whose isoelectric point is comprised from 5.8 to 8.5, a solution of GLP-1 RA, an analog or a derivative of GLP-1 RA and a co-polyamino acid bearing carboxylate charges and at least one hydrophobic radical according to the invention, as an aqueous solution or in freeze-dried form. If necessary, the pH of the preparation is adjusted to a pH comprised between 6 and 8.

The preparation of a composition according to the invention has the advantage of being able to be achieved by simple mixing of an aqueous solution of basal insulin whose isoelectric point is comprised from 5.8 to 8.5, a solution of prandial insulin, a solution of GLP-1 RA or an analog or a derivative of GLP-1 RA and a co-polyamino acid bearing carboxylate charges and at least one hydrophobic radical according to the invention, as an aqueous solution or in freeze-dried form. If necessary, the pH of the preparation is adjusted to a pH comprised between 6 and 8.

In one embodiment, the basal insulin and co-polyamino acid mixture is concentrated by ultrafiltration before mixing with prandial insulin in aqueous solution or freeze-dried form.

If necessary, the mixture composition is adjusted to excipients such as glycerin, m-cresol, zinc chloride, and polysorbate (Tween®) by adding concentrated solutions of these excipients to the mixture. If necessary, the pH of the preparation is adjusted to a pH comprised between 6 and 8.

The invention also relates to compositions which further comprise ionic species, the said ionic species improve the stability of the compositions.

The invention also relates to the use of ionic species chosen from the group of anions, cations and/or zwitterions to improve the physicochemical stability of the compositions.

In one embodiment, the ionic species comprise less than 10 carbon atoms.

The said ionic species are chosen from the group of anions, cations and/or zwitterions. Zwitterion refers to a species bearing at least one positive charge and at least one negative charge on two non-adjacent atoms.

The said ionic species are used alone or as a mixture and preferably as a mixture.

In one embodiment, the anions are selected from organic anions.

In one embodiment the organic anions comprise less than 10 carbon atoms.

In one embodiment, the organic anions are chosen from the group consisting of acetate, citrate and succinate

In one embodiment, the anions are selected from anions of mineral origin.

In one embodiment, the anions of mineral origin are chosen from the group consisting of sulphates, phosphates and halides, especially chlorides.

In one embodiment, the cations are selected from organic cations.

In one embodiment, the organic cations comprise less than 10 carbon atoms.

In one embodiment, the organic cations are chosen from the group consisting of ammoniums, like 2-Amino-2-(hydroxymethyl) propane-1,3-diol wherein the amine is in ammonium form.

In one embodiment, cations are selected from cations of mineral origin.

In one embodiment the cations of mineral origin are chosen from the group consisting of zinc, in particular Zn²⁺ and alkaline metals, in particular Na⁺ and K⁺,

In one embodiment, zwitterions are selected from zwitterions of organic origin.

In one embodiment, zwitterions of organic origin are selected from amino acids.

In one embodiment amino acids are selected from aliphatic amino acids in the group consisting of glycine, alanine, valine, isoleucine and leucine.

In one embodiment, amino acids are selected from cyclic amino acids in the group consisting of proline.

In one embodiment the amino acids are selected from hydroxylated or sulfur amino acids in the group consisting of cysteine, serine, threonine, and methionine.

In one embodiment, amino acids are selected from aromatic amino acids in the group consisting of phenylalanine, tyrosine and tryptophan.

In one embodiment, amino acids are selected from amino acids whose carboxyl function of the side chain is amidified in the group consisting of asparagine and glutamine.

In one embodiment, zwitterions of organic origin are chosen from the group consisting of amino acids having an uncharged side chain.

In one embodiment, zwitterions of organic origin are chosen from the group consisting of aminodiacides or acidic amino acids.

In one embodiment, aminodiacides are chosen from the group consisting of glutamic acid and aspartic acid, possibly in the form of salts.

In one embodiment, zwitterions of organic origin are chosen from the group consisting of basic or so-called “cationic” amino acids.

In one embodiment, “cationic” amino acids are selected from arginine, histidine and lysine, especially arginine and lysine.

In particular, zwitterions comprise as many negative charges as positive charges and therefore a nil overall charge at the isoelectric point and/or at a pH between 6 and 8.

Said ionic species are introduced into the compositions in the form of salts. The introduction of these can be in solid form before dissolution in the compositions, or in the form of a solution, in particular of concentrated solution.

For example, mineral-based cations are provided in the form of salts selected from sodium chloride, zinc chloride, sodium phosphate, sodium sulfate, etc.

For example, anions of organic origin are provided in the form of salts selected from sodium or potassium citrate, sodium acetate.

For example, amino acids are added in the form of salts selected from arginine hydrochloride, histidine hydrochloride or in non-salified form, for example histidine or arginine.

In one embodiment the ionic species is selected from sodium chloride, sodium citrate, and zinc chloride.

In one embodiment, the total molar concentration of ionic species in the composition is greater than or equal to 10 mM.

In one embodiment, the total molar concentration of ionic species in the composition is greater than or equal to 20 mM.

In one embodiment, the total molar concentration of ionic species in the composition is greater than or equal to 30 mM.

In one embodiment, the total molar concentration of ionic species in the composition is greater than or equal to 50 mM.

In one embodiment, the total molar concentration of ionic species in the composition is greater than or equal to 75 mM.

In one embodiment, the total molar concentration of ionic species in the composition is greater than or equal to 100 mM.

In one embodiment, the total molar concentration of ionic species in the composition is greater than or equal to 200 mM.

In one embodiment, the total molar concentration of ionic species in the composition is greater than or equal to 300 mM.

In one embodiment, the total molar concentration of ionic species in the composition is greater than or equal to 500 mM.

In one embodiment, the total molar concentration of ionic species in the composition is greater than or equal to 600 mM.

In one embodiment, the total molar concentration of ionic species in the composition is greater than or equal to 700 mM.

In one embodiment, the total molar concentration of ionic species in the composition is greater than or equal to 800 mM.

In one embodiment, the total molar concentration of ionic species in the composition is greater than or equal to 900 mM.

In one embodiment, the total molar concentration of ionic species in the composition is less than or equal to 1000 mM.

In one embodiment, the total molar concentration of ionic species in the composition is less than or equal to 1500 mM.

In one embodiment, the total molar concentration of ionic species in the composition is less than or equal to 1200 mM.

In one embodiment, the total molar concentration of ionic species in the composition is less than or equal to 1000 mM.

In one embodiment, the total molar concentration of ionic species in the composition is less than or equal to 900 mM.

In one embodiment, the total molar concentration of ionic species in the composition is less than or equal to 800 mM.

In one embodiment, the total molar concentration of ionic species in the composition is less than or equal to 700 mM.

In one embodiment, the total molar concentration of ionic species in the composition is less than or equal to 600 mM.

In one embodiment, the total molar concentration of ionic species in the composition is less than or equal to 500 mM.

In one embodiment, the total molar concentration of ionic species in the composition is less than or equal to 400 mM.

In one embodiment, the total molar concentration of ionic species in the composition is less than or equal to 300 mM.

In one embodiment, the total molar concentration of ionic species in the composition is less than or equal to 200 mM.

In one embodiment, the total molar concentration of ionic species in the composition is less than or equal to 100 mM.

In one embodiment the total molar concentration of ion species in the composition is comprised from 10 to 1000 mM.

In one embodiment the total molar concentration of ion species in the composition is comprised from 20 to 1000 mM.

In one embodiment the total molar concentration of ion species in the composition is comprised from 30 to 1000 mM.

In one embodiment the total molar concentration of ion species in the composition is comprised from 50 to 1000 mM.

In one embodiment the total molar concentration of ion species in the composition is comprised from 75 to 1000 mM.

In one embodiment the total molar concentration of ion species in the composition is comprised from 100 to 1000 mM.

In one embodiment the total molar concentration of ion species in the composition is comprised from 200 to 1000 mM.

In one embodiment the total molar concentration of ion species in the composition is comprised from 300 to 1000 mM.

In one embodiment the total molar concentration of ion species in the composition is comprised from 400 to 1000 mM.

In one embodiment the total molar concentration of ion species in the composition is comprised from 500 to 1000 mM.

In one embodiment the total molar concentration of ion species in the composition is comprised from 600 to 1000 mM.

In one embodiment the total molar concentration of ion species in the composition is comprised from 10 to 900 mM.

In one embodiment the total molar concentration of ion species in the composition is comprised from 20 to 900 mM.

In one embodiment the total molar concentration of ion species in the composition is comprised from 30 to 900 mM.

In one embodiment the total molar concentration of ion species in the composition is comprised from 50 to 900 mM.

In one embodiment the total molar concentration of ion species in the composition is comprised from 75 to 900 mM.

In one embodiment the total molar concentration of ion species in the composition is comprised from 100 to 900 mM.

In one embodiment the total molar concentration of ion species in the composition is comprised from 200 to 900 mM.

In one embodiment the total molar concentration of ion species in the composition is comprised from 300 to 900 mM.

In one embodiment the total molar concentration of ion species in the composition is comprised from 400 to 900 mM.

In one embodiment the total molar concentration of ion species in the composition is comprised from 500 to 900 mM.

In one embodiment the total molar concentration of ion species in the composition is comprised from 600 to 900 mM.

In one embodiment the total molar concentration of ion species in the composition is comprised from 10 to 800 mM.

In one embodiment the total molar concentration of ion species in the composition is comprised from 20 to 800 mM.

In one embodiment the total molar concentration of ion species in the composition is comprised from 30 to 800 mM.

In one embodiment the total molar concentration of ion species in the composition is comprised from 50 to 800 mM.

In one embodiment the total molar concentration of ion species in the composition is comprised from 75 to 800 mM.

In one embodiment the total molar concentration of ion species in the composition is comprised from 100 to 800 mM.

In one embodiment the total molar concentration of ion species in the composition is comprised from 200 to 800 mM.

In one embodiment the total molar concentration of ion species in the composition is comprised from 300 to 800 mM.

In one embodiment the total molar concentration of ion species in the composition is comprised from 400 to 800 mM.

In one embodiment the total molar concentration of ion species in the composition is comprised from 500 to 800 mM.

In one embodiment the total molar concentration of ion species in the composition is comprised from 600 to 800 mM.

In one embodiment the total molar concentration of ion species in the composition is comprised from 10 to 700 mM.

In one embodiment the total molar concentration of ion species in the composition is comprised from 20 to 700 mM.

In one embodiment the total molar concentration of ion species in the composition is comprised from 30 to 700 mM.

In one embodiment the total molar concentration of ion species in the composition is comprised from 50 to 700 mM.

In one embodiment the total molar concentration of ion species in the composition is comprised from 75 to 700 mM.

In one embodiment the total molar concentration of ion species in the composition is comprised from 100 to 700 mM.

In one embodiment the total molar concentration of ion species in the composition is comprised from 200 to 700 mM.

In one embodiment the total molar concentration of ion species in the composition is comprised from 300 to 700 mM.

In one embodiment the total molar concentration of ion species in the composition is comprised from 400 to 700 mM.

In one embodiment the total molar concentration of ion species in the composition is comprised from 500 to 700 mM.

In one embodiment the total molar concentration of ion species in the composition is comprised from 600 to 700 mM.

In one embodiment the total molar concentration of ion species in the composition is comprised from 10 to 600 mM.

In one embodiment the total molar concentration of ion species in the composition is comprised from 20 to 600 mM.

In one embodiment the total molar concentration of ion species in the composition is comprised from 30 to 600 mM.

In one embodiment the total molar concentration of ion species in the composition is comprised from 50 to 600 mM.

In one embodiment the total molar concentration of ion species in the composition is comprised from 75 to 600 mM.

In one embodiment the total molar concentration of ion species in the composition is comprised from 100 to 600 mM.

In one embodiment the total molar concentration of ion species in the composition is comprised from 200 to 600 mM.

In one embodiment the total molar concentration of ion species in the composition is comprised from 300 to 600 mM.

In one embodiment the total molar concentration of ion species in the composition is comprised from 400 to 600 mM.

In one embodiment the total molar concentration of ion species in the composition is comprised from 500 to 600 mM.

In one embodiment the total molar concentration of ion species in the composition is comprised from 10 to 500 mM.

In one embodiment the total molar concentration of ion species in the composition is comprised from 20 to 500 mM.

In one embodiment the total molar concentration of ion species in the composition is comprised from 30 to 500 mM.

In one embodiment the total molar concentration of ion species in the composition is comprised from 50 to 500 mM.

In one embodiment the total molar concentration of ion species in the composition is comprised from 75 to 500 mM.

In one embodiment the total molar concentration of ion species in the composition is comprised from 100 to 500 mM.

In one embodiment the total molar concentration of ion species in the composition is comprised from 200 to 500 mM.

In one embodiment the total molar concentration of ion species in the composition is comprised from 300 to 500 mM.

In one embodiment the total molar concentration of ion species in the composition is comprised from 400 to 500 mM.

In one embodiment the total molar concentration of ion species in the composition is comprised from 10 to 400 mM.

In one embodiment the total molar concentration of ion species in the composition is comprised from 20 to 400 mM.

In one embodiment the total molar concentration of ion species in the composition is comprised from 30 to 400 mM.

In one embodiment the total molar concentration of ion species in the composition is comprised from 50 to 400 mM.

In one embodiment the total molar concentration of ion species in the composition is comprised from 75 to 400 mM.

In one embodiment the total molar concentration of ion species in the composition is comprised from 100 to 400 mM.

In one embodiment the total molar concentration of ion species in the composition is comprised from 200 to 400 mM.

In one embodiment the total molar concentration of ion species in the composition is comprised from 300 to 400 mM.

In one embodiment the total molar concentration of ion species in the composition is comprised from 10 to 300 mM.

In one embodiment the total molar concentration of ion species in the composition is comprised from 20 to 300 mM.

In one embodiment the total molar concentration of ion species in the composition is comprised from 30 to 300 mM.

In one embodiment the total molar concentration of ion species in the composition is comprised from 50 to 300 mM.

In one embodiment the total molar concentration of ion species in the composition is comprised from 75 to 300 mM.

In one embodiment the total molar concentration of ion species in the composition is comprised from 100 to 300 mM.

In one embodiment the total molar concentration of ion species in the composition is comprised from 200 to 300 mM.

In one embodiment the total molar concentration of ion species in the composition is comprised from 10 to 200 mM.

In one embodiment the total molar concentration of ion species in the composition is comprised from 20 to 200 mM.

In one embodiment the total molar concentration of ion species in the composition is comprised from 30 to 200 mM.

In one embodiment the total molar concentration of ion species in the composition is comprised from 50 to 200 mM.

In one embodiment the total molar concentration of ion species in the composition is comprised from 75 to 200 mM.

In one embodiment the total molar concentration of ion species in the composition is comprised from 100 to 200 mM.

In one embodiment the total molar concentration of ion species in the composition is comprised from 10 to 100 mM.

In one embodiment the total molar concentration of ion species in the composition is comprised from 20 to 100 mM.

In one embodiment the total molar concentration of ion species in the composition is comprised from 30 to 100 mM.

In one embodiment the total molar concentration of ion species in the composition is comprised from 50 to 100 mM.

In one embodiment the total molar concentration of ion species in the composition is comprised from 75 to 100 mM.

In one embodiment the total molar concentration of ion species in the composition is comprised from 10 to 75 mM.

In one embodiment the total molar concentration of ion species in the composition is comprised from 20 to 75 mM.

In one embodiment the total molar concentration of ion species in the composition is comprised from 30 to 75 mM.

In one embodiment the total molar concentration of ion species in the composition is comprised from 50 to 75 mM.

In one embodiment the total molar concentration of ion species in the composition is comprised from 10 to 50 mM.

In one embodiment the total molar concentration of ion species in the composition is comprised from 20 to 50 mM.

In one embodiment the total molar concentration of ion species in the composition is comprised from 30 to 50 mM.

In one embodiment, said ionic species are present in a concentration ranging from 5 to 400 mM.

In one embodiment, said ionic species are present in a concentration ranging from 5 to 300 mM.

In one embodiment, said ionic species are present in a concentration ranging from 5 to 200 mM.

In one embodiment, said ionic species are present in a concentration ranging from 5 to 100 mM.

In one embodiment, said ionic species are present in a concentration ranging from 5 to 75 mM.

In one embodiment, said ionic species are present in a concentration ranging from 5 to 50 mM.

In one embodiment, said ionic species are present in a concentration ranging from 5 to 25 mM.

In one embodiment, said ionic species are present in a concentration ranging from 5 to 20 mM.

In one embodiment, said ionic species are present in a concentration ranging from 5 to 10 mM.

In one embodiment, said ionic species are present in a concentration ranging from 10 to 400 mM.

In one embodiment, said ionic species are present in a concentration ranging from 10 to 300 mM.

In one embodiment, said ionic species are present in a concentration ranging from 10 to 200 mM.

In one embodiment, said ionic species are present in a concentration ranging from 10 to 100 mM.

In one embodiment, said ionic species are present in a concentration ranging from 10 to 75 mM.

In one embodiment, said ionic species are present in a concentration ranging from 10 to 50 mM.

In one embodiment, said ionic species are present in a concentration ranging from 10 to 25 mM.

In one embodiment, said ionic species are present in a concentration ranging from 10 to 20 mM.

In one embodiment, said ionic species are present in a concentration ranging from 20 to 300 mM.

In one embodiment, said ionic species are present in a concentration ranging from 20 to 200 mM.

In one embodiment, said ionic species are present in a concentration ranging from 20 to 100 mM.

In one embodiment, said ionic species are present in a concentration ranging from 20 to 75 mM.

In one embodiment, said ionic species are present in a concentration ranging from 20 to 50 mM.

In one embodiment, said ionic species are present in a concentration ranging from 20 to 25 mM.

In one embodiment, said ionic species are present in a concentration ranging from 50 to 300 mM.

In one embodiment, said ionic species are present in a concentration ranging from 50 to 200 mM.

In one embodiment, said ionic species are present in a concentration ranging from 50 to 100 mM.

In one embodiment, said ionic species are present in a concentration ranging from 50 to 75 mM.

Regarding cations of mineral origin, and in particular Zn²⁺, its molar concentration in the composition may be between 0.25 and 20 mM, in particular between 0.25 and 10 mM or between 0.25 and 5 mM.

In one embodiment, the composition comprises zinc.

In one embodiment, the composition comprises from 0.2 to 2 mM of Zinc.

In one embodiment, the composition comprises NaCl.

In one embodiment NaCl is present in a concentration ranging from 2 to 25 mM

In one embodiment NaCl is present in a concentration ranging from 2.5 to 20 mM

In one embodiment NaCl is present in a concentration ranging from 4 to 15 mM

In one embodiment NaCl is present in a concentration ranging from 5 to 10 mM

### Part A

### AA: Synthesis of Intermediate Hydrophobic Compounds Hy to Obtain the -Hy Radicals Wherein p=1

The hydrophobic intermediate compounds are represented in the following table by the corresponding hydrophobic molecule before co-polyamino acid grafting.

TABLE 1A
list and structures of the hydrophobic molecules synthesized according to the invention.

| No | HYDROPHOBIC INTERMEDIATE COMPOUNDS |
|---|---|
| AA1 | 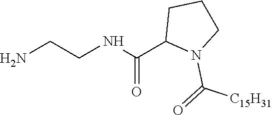 |
| AA2 | 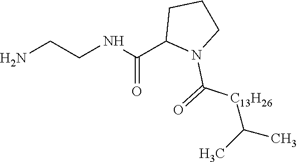 |
| AA3 | 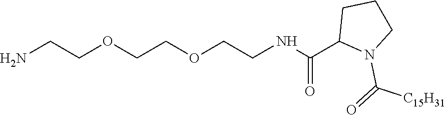 |
| AA4 | 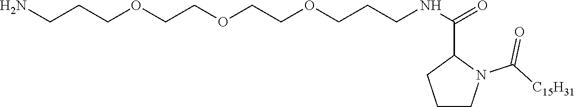 |
| AA5 | 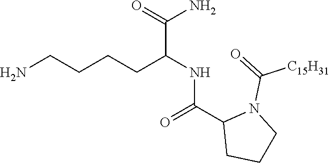 |
| AA6 | 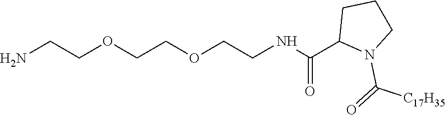 |
| AA7 | 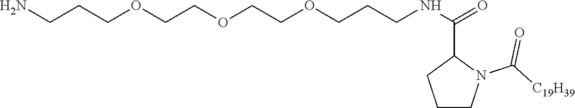 |
| AA8 | 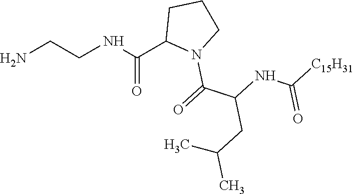 |
| AA9 | 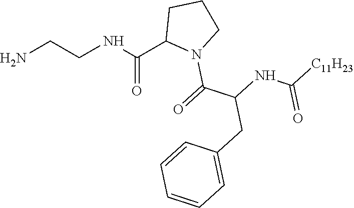 |
| AA10 | 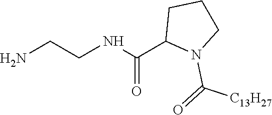 |
| AA11 | 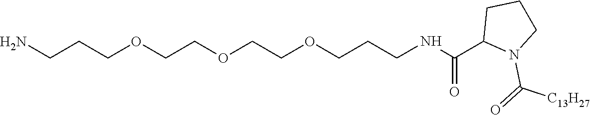 |
| AA12 | 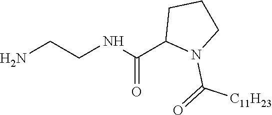 |
| AA13 | 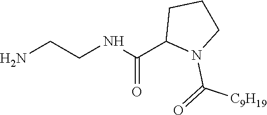 |
| AA14 | 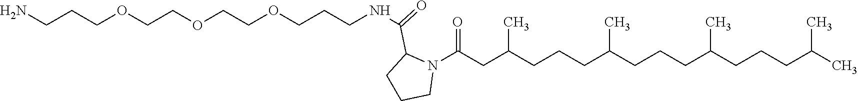 [SMILES: CC(C)CCCC(C)CCCC(C)CCCC(C)CC(=O)N1CCCC1C(=O)NCCCOCCOCCOCCCN] |
| AA15 | 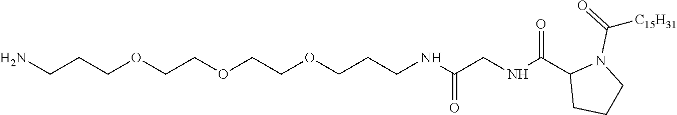 |
| AA16 | 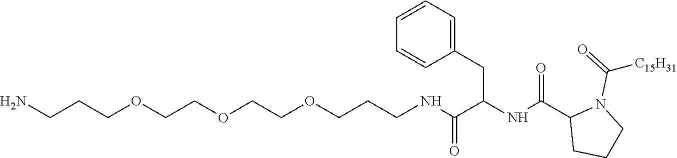 |
| AA17 | 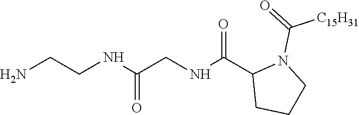 |
| AA18 | 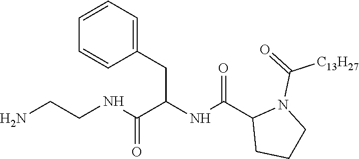 |

### EXAMPLE AA1: MOLECULE AA1

### Molecule A1: Product Obtained by the Reaction Between Palmitoyl Chloride and L-Proline.

A solution of palmitoyl chloride (23.0 g, 83.7 mmol) in acetone (167 mL) is added dropwise over 90 minutes to a solution of L-proline (10.6 g, 92.1 mmol) in 1 N aqueous sodium hydroxide

(230 mL; 230 mmol). After stirring for 14 hours at room temperature, the heterogeneous mixture is cooled to 0° C., then filtered on a sintered frit to give a white solid which is washed with water (2×100 mL), then diisopropyl ether (100 mL). The solid is dried under reduced pressure. The solid is then dissolved under reflux in 200 mL of water, then 8 mL of a 37% hydrochloric acid solution are added to obtain a pH=1. The opalescent reaction medium is then cooled to 0° C. The precipitate obtained is filtered on sintered, then washed with water (5×50 mL) until filtrates of physiological pH between 6.0 and 8.0 are obtained, then dried in an oven at 50° C. under vacuum overnight. The product is purified by recrystallization in diisopropyl ether. A white solid is obtained.

Yield: 22.7 g (77%).

NMR ¹H (CDCl₃, ppm): 0.88 (3H); 1.19-1.45 (24H); 1.58-1.74 (2H); 1.88-2.14 (3H); 2.15-2.54 (3H); 3.47 (1H); 3.58 (1H); 4.41 (0.1H); 4.61 (0.9H) 6.60-8.60 (1H).

### Molecule A2: Product Obtained by Reaction Between Molecule A1 and N-Boc-Ethylenediamine.

N,N-diisopropylethylamine (DIPEA) (68.8 g, 532.3 mmol), 1-hydroxybenzotriazole (HOBt) (37.1 g, 274.6 mmol), then N(3-dimethylaminopropyl)-N′-ethylcarbodiimide (EDC) (53.1 g, 277.0 mmol) are successively added at room temperature to a solution of molecule A1 (75.1 g, 212.4 mmol) in 1500 mL of chloroform. After stirring for 15 minutes at room temperature, a solution of N-Boc-ethylenediamine (BocEDA) (37.6 g, 234.7 mmol) in 35 mL of chloroform is added. After stirring for 18 hours at room temperature, a solution of 0.1 N HCl (2.1 L), then a saturated solution of NaCl (1 L) are added. The phases are separated then the organic phase is washed successively with a solution of 0.1 N HCl/saturated NaCl (2.1 L/L), a saturated solution of NaCl (2 L), a saturated NaHCO₃ (2 L) solution, then a saturated NaCl (2 L) solution. The organic phase is dried over anhydrous sodium sulphate, filtered, then concentrated under reduced pressure. The solid obtained is purified by trituration in diisopropyl ether (3×400 mL), to yield a solid after drying under vacuum at 40° C.

Yield: 90.4 g (86%).

NMR ¹H (CDCl₃, ppm): 0.88 (3H); 1.20-1.37 (24H); 1.44 (9H); 1.54-1.70 (2H); 1.79-1.92 (1H); 1.92-2.04 (1H); 2.03-2.17 (1H); 2.17-2.44 (3H); 3.14-3.36 (4H); 3.43 (1H); 3.56 (1H); 4.29 (0.1 H); 4.51 (0.9 H); 4.82 (0.1H); 5.02 (0.9H); 6.84 (0.1H); 7.22 (0.9H).

### Molecule AA1

A solution of 4 N hydrochloric acid in dioxane (100 mL, 400 mmol) is added dropwise and at 0° C. to a solution molecule A2 (20.1 g, 40.5 mmol) in 330 mL of dichloromethane. After stirring for 3 hours 30 minutes at room temperature, the solution is concentrated under reduced pressure. The residue is purified by flash chromatography (methanol, dichloromethane) to yield a white solid of molecule AA1 in the form of a hydrochloride salt.

Yield: 16.3 g (93%).

NMR ¹H (CDCl₃, ppm): 0.88 (3H); 1.07-1.40 (24H); 1.49-1.63 (2H); 1.77-2.18 (4H); 2.18-2.45 (2H); 3.14-3.32 (2H); 3.42-3.63 (2H); 3.63-3.84 (2H); 4.37 (0.1H); 4.48 (0.9H); 6.81-8.81 (4H).

LC/MS (ESI): 396.5; (calculated ([M+H]⁺): 396.4).

### EXAMPLE AA2: MOLECULE AA2

Molecule A3: 15-methylhexadecan-1-ol.

Magnesium in chips (9.46 g, 389 mmol) is introduced into a three-neck flask under argon. The magnesium is covered with anhydrous THF (40 mL), and a few drops of I-bromo-3-methylbutane are added at room temperature to initiate the reaction. After the observation of an exotherm and a slight turbidity of the medium, the rest of the 1-bromo-3-methylbutane (53.87 g, 357 mmol) is added dropwise over 90 minutes while the temperature of the medium remains stable between 50 and 60° C. The reaction medium is then heated at 70° C. for 2 hours.

In a three-necked flask under argon, a solution of 12-bromo-1-dodecanol (43 g, 162.1 mmol) in THE (60 mL) is added dropwise at 0° C. to a solution of CuCl (482 mg, 4.86 mmol) dissolved in NMP (62 mL). To this solution is then added dropwise, the hot organomagnesium solution, freshly prepared in order to maintain the temperature of the medium below 20° C. The mixture is then stirred at ambient temperature for 16 hours. The medium is cooled to 0° C. and the reaction is stopped by addition of a 1 N HCl aqueous solution to pH 1 and the medium is extracted with ethyl acetate. After washing the organic phase with saturated NaCl solution and drying over Na₂SO₄, the solution is filtered and concentrated under vacuum to produce an oil. After purification by DCVC on silica gel (cyclohexane, ethyl acetate), an oil which crystallizes at room temperature is obtained.

Yield: 32.8 g (74%)

NMR ¹H (CDCl₃, ppm): 0.87 (6H); 1.14 (2H); 1.20-1.35 (22H); 1.50-1.55 (3H); 3.64 (2H).

Molecule A4: 15-methylbexadecanoic acid.

To a solution of molecule A3 (20.65 g, 80.5 mmol) and tetrabutylammonium bromide (14.02 g, 42.5 mmol) in a mixture of acetic acid/dichloroethane/water (124/400/320 mL) at room temperature is added in small portions of potassium permanganate (38.2 g, 241.5 mmol). After stirring under reflux for 5 hours and return to room temperature, the medium is acidified to pH 1 by progressive addition of 5N HCl. Na₂SO₃ (44.6 g, 354.3 mmol) is then gradually added until the medium is bleached. The aqueous phase is extracted with dichloromethane, and the combined organic phases are dried over Na₂SO₄, filtered and concentrated under vacuum. After purification by chromatography on silica gel (cyclohexane, ethyl acetate, acetic acid), a white solid is obtained.

Yield: 19.1 g (quantitative)

NMR ¹H (CDCl₃, ppm): 0.87 (6H); 1.14 (2H); 1.22-1.38 (20H); 1.51 (1H); 1.63 (2H); 2.35 (2H).

### Molecule A5: Product Obtained by Reaction Between the A4 Molecule and L-Proline.

Dicyclohexyl carbodiimide (DCC) (8.01 g, 38.8 mmol) and N-hydroxysuccinimide (NHS) (4.47 g, 38.8 mmol) are successively added to a solution of molecule A4 (10 g, 37 mmol) in THF (360 mL) at 0° C. After stirring for 17 hours at room temperature, the medium is cooled to 0° C. for 20 minutes, filtered on sinter. L-Proline (4 g, 37.7 mmol), triethylamine (34 mL) and water (30 mL) are added to the filtrate. After stirring for 20 hours at room temperature, the medium is treated with a 1N HCl aqueous solution until pH 1. The aqueous phase is extracted with dichloromethane (2×125 mL). The combined organic phases are washed with an aqueous solution of 1 N HCl (2×100 mL), water (100 mL), then a saturated aqueous solution of NaCl (100 mL). After drying over Na₂SO₄, the organic phase is filtered, concentrated under vacuum, and the residue is purified by chromatography on silica gel (cyclohexane, ethyl acetate, acetic acid)

Yield: 9.2 g (72%)

NMR ¹H (CDCl₃, ppm): 0.86 (6H); 1.14 (2H); 1.22-1.38 (20H); 1.50 (1H); 1.67 (2H); 1.95-2.10 (311); 2.34 (2H); 2.49 (1H); 3.47 (1H); 3.56 (1H); 4.61 (1H).

LC/MS (ESI): 368.3; (calculated ([M+H]⁺): 368.6).

### Molecule A6: Product Obtained by Reaction Between Molecule A5 and N-Boc-Ethylenediamine.

Triethylamine (TEA) (5.23 mL) and 2-(1H-benzotriazol-1-yl)-1,1,3,3-tetramethyluronium tetrafluoroborate (TBTU) are added to a solution of molecule A5 (9.22 g, 25.08 mmol) in a THF/DMF mixture (200/50 mL) at room temperature. After 10 minutes of stirring, BocEDA (4.42 g, 27.6 mmol) is added. After stirring at room temperature for 17 h, the mixture is diluted with water (300 mL) at 0° C. and stirred cold for 20 minutes. The precipitate formed is sintered and the filtrate is extracted with ethyl acetate. The combined organic phases are washed with saturated NaHCO₃, solution, dried on Na₂SO₄, filtered, concentrated under vacuum and the residue purified by flash chromatography (ethyl acetate, methanol).

Yield: 6.9 g (54%)

NMR ¹H (CDCl₃, ppm): 0.86 (6H); 1.15 (2H); 1.22-1.38 (20H); 1.43 (911); 1.50 (1H); 1.64 (4H); 1.85 (1H); 1.95 (1H); 2.10 (1H); 2.31 (2H); 3.20-3.35 (3H); 3.45 (1H); 3.56 (1H); 4.51 (1H); 5.05 (1H); 7.24 (1H).

LC/MS (ESI): 510.6; (calculated ([M+H]⁺): 510.8).

### Molecule AA2

A 4 N HCl solution in dioxane (13 mL) is added to a molecule A6 (5.3 g, 10.40 mmol) solution in dichloromethane (50 mL) at 0° C. After stirring for 5 hours at 0° C., the medium is concentrated under vacuum, returned to water and freeze-dried to give a white solid of molecule AA2 in the form of hydrochloride salt.

Yield: 4.6 g (99%)

NMR ¹H (D₂O, ppm): 0.91 (6H); 1.22 (2H); 1.22-1.50 (20H); 1.63 (3H); 1.98 (1H); 2.10 (2H); 2.26 (1H); 2.39 (1H); 2.43 (1H); 3.22 (2H); 3.45-3.60 (3H); 3.78 (1H); 4.42 (11H).

LC/MS (ESI): 410.4; (calculated ([M+H]⁺): 410.7).

### EXAMPLE AA3: MOLECULE AA3

Molecule A7: Product Obtained by the Reaction Between Molecule A1 and Boc-Tri(Ethylene Glycol)Diamine.

By a process similar to that used in the preparation of molecule A2 applied to molecule A1 (4.0 g, 11.3 mmol) and Boc-tri (ethylene glycol) diamine (3.1 g, 12.4 mmol), a colorless oil is obtained after purification by flash chromatography (methanol, toluene).

Yield: 5.5 g (84%).

NMR ¹H (CDCl, ppm): 0.88 (3H); 1.09-1.39 (24H); 1.44 (9H); 1.64 (2H); 1.79-2.01 (2H); 2.06-2.43 (4H); 3.23-3.68 (14H); 4.33 (0.2H); 4.56 (0.8H); 5.25 (1H); 6.49 (0.2H); 7.13-7.50 (0.811).

### Molecule AA3

By a process similar to the one used in the preparation of molecule AA1 applied to molecule A7 (5.5 g, 9.4 mmol), a white solid of molecule AA3 in the form of a hydrochloride salt is obtained after purification by flash chromatography (methanol, dichloromethane).

Yield: 4.3 g (92%).

NMR ¹H (DMSO-d6, ppm): 0.85 (3H); 1.08-1.40 (24H); 1.40-1.52 (2H); 1.71-2.02 (4H); 2.02-2.31 (2H); 2.90-2.98 (2H); 3.15-3.47 (5H); 3.50-3.66 (7H); 4.24 (0.6H); 4.32 (0.4H); 7.83 (0.6H); 7.95 (3H); 8.17 (0.4H).

LC/MS (ESI): 484.6; (calculated ([M+H]⁺): 484.4).

### EXAMPLE AA4: MOLECULE AA4

Molecule A8: Product Obtained by Reaction Between Molecule A1 and Boc-1-Amino-4,7,10-trioxa-13-tridecane amine.

By a process similar to the one used in the preparation of molecule A2 applied to molecule A1 (4.5 g, 12.7 mmol) and to Boc-1-amino-4,7,10-trioxa-13-tridecane amine (4.5 g, 14.0 mmol), a yellow oil is obtained after purification by flash chromatography (methanol, dichloromethane).

Yield: 7.7 g (92%).

NMR ¹H (CDCl₃, ppm): 0.88 (3H); 1.22-1.37 (24H); 1.44 (9H); 1.59-1.67 (2H); 1.67-2.00 (6H); 2.06-2.45 (4H); 3.18-3.76 (18H); 4.28 (0.2H); 4.52 (0.8H); 4.69-5.04 (1H); 6.77 (0.2H); 7.20 (0.8H).

### Molecule AA4

By a process similar to the one used in the preparation of molecule AA1 applied to molecule A8 (7.7 g, 11.8 mmol), a yellow oil is obtained after purification by flash chromatography (methanol, dichloromethane). A co-evaporation with diisopropyl ether facilitates the obtention of molecule AA4 in the form of a hydrochloride salt in the form of a white solid which is dried under vacuum at 50° C.

Yield: 5.4 g (76%).

NMR ¹H (CDCl₃, ppm): 0.88 (3H); 1.08-1.40 (24H); 1.49-1.65 (2H); 1.76-2.39 (10H); 3.07-3.28 (3H); 3.34-3.80 (15H); 4.34 (0.05H); 4.64 (0.95H); 7.35 (0.05H); 7.66-8.58 (3.95H).

LC/MS (ESI): 556.7; (calculated ([M+H]⁺): 556.5).

### EXAMPLE AA5: MOLECULE AA5

Molecule A9: Product Obtained by Reaction Between Molecule A1 and the Methyl Ester of N-Boc-L-Lysine.

By a process similar to that used for the preparation of molecule A2 applied to molecule A1 (4 g, 11.3 mmol) and methyl ester of N-Boc-L-lysine (3.2 g, 12.4 mmol), a colorless oil is obtained after purification by flash chromatography (methanol, dichloromethane).

Yield: 4.9 g (73%).

NMR ¹H (CDCl₃, ppm): 0.88 (3H); 0.99-1.54 (37H); 1.54-1.75 (3H); 1.75-2.04 (3H); 2.04-2.41 (4H); 2.94-3.19 (2H); 3.19-3.81 (5H); 4.28-4.64 (2H); 4.94 (1H); 6.45 (0.1H); 7.36 (0.9H).

LC/MS (ESI): 596.7; (calculated ([M+H]⁺): 596.5).

Molecule A10: Product Obtained by Treatment of Molecule A9 with Ammonia.

320 mL of a 7 N ammonia solution in methanol are added to a suspension of molecule A9 (4.9 g, 8.2 mmol) in 10 mL of methanol. After stirring for 19 hours at room temperature in a closed atmosphere, an additional 100 ml of ammonia solution are added. After stirring for 24 hours at room temperature in a closed atmosphere, the reaction medium is concentrated under reduced pressure. The residue is purified by trituration in refluxing diisopropyl ether (100 mL) to give a white solid which is dried under vacuum at 50° C.

Yield: 4.1 g (85%).

NMR ¹H (CDCl₃, ppm): 0.88 (31-1); 1.06-1.57 (37H); 1.57-1.79 (3H); 1.88-2.41 (7H); 3.09 (2H); 3.49 (1H); 3.62 (1H); 4.34 (1H); 4.51 (1H); 4.69-4.81 (1H); 5.43 (0.95H); 5.57 (0.05H); 6.25 (0.05H); 6.52 (0.95H); 6.83 (0.05H); 7.11 (0.95H).

### Molecule AA5

By a process similar to the one used in the preparation of molecule AA1 applied to molecule A10 (388 mg, 0.67 mmol), a white solid of molecule AA5 in the form of a hydrochloride salt is obtained after purification by trituration in diisopropyl ether.

Yield: 292 mg (85%).

NMR ¹H (DMSO-d6, ppm): 0.85 (3H); 1.06-2.34 (38H); 2.61-2.81 (211); 3.29-3.68 (2H); 4.05-4.17 (1.7H); 4.42 (0.3H); 7.00 (1H); 7.16 (0.7H); 7.43 (0.3H); 7.73-8.04 (3.7H); 8.16 (0.3H). LC/MS (ESI): 481.6; (calculated ([M+H]⁺): 481.4).

### EXAMPLE AA6: MOLECULE AA6

### Molecule A11: Product Obtained by the Reaction Between Stearoyl Chloride and L-Proline.

By a process similar to the one used in the preparation of molecule A1 applied to L-proline (5.0 g, 43.4 mmol) and to stearoyl chloride (12.0 g, 39.6 mmol), a white solid is obtained after purification by flash chromatography (methanol, dichloromethane).

Yield: 5.37 g (36%)

NMR ¹H (CDCl₃, ppm): 0.88 (3H); 1.26-1.37 (28H); 1.64-1.70 (2H); 1.88-2.10 (3H); 2.36 (2H); 2.54-2.58 (1H); 3.46 (1H); 3.56 (1H); 4.62 (1H).

LC/MS (ESI): 382.6; (calculated ([M+H]+): 382.3).

### Molecule A12: Product Obtained by Reaction Between Molecule all and Boc-Tri(Ethylene Glycol)Diamine.

By a process similar to that used in the preparation of molecule A6 applied to molecule A11 (33.81 g, 88.6 mmol) and Boc-tri (ethylene glycol) diamine (26.4 g, 106.3 mmol) in THF using DIPEA instead of TEA, a white solid is obtained after purification by flash chromatography (ethyl acetate, methanol).

Yield: 43.3 g (80%)

NMR ¹H (CDCl₃, ppm): 0.87 (3H); 1.24 (30H); 1.43 (9H); 1.61 (2H); 1.82 (1H); 1.96 (1H); 2.25-2.45 (2H); 3.25-3.65 (14H); 4.30 (0.15H); 4.53 (0.85H); 5.25 (1H); 6.43 (0.15H); 7.25 (0.85H).

LC/MS (ESI): 612.6; (calculated ([M+H]⁺): 612.9).

### Molecule AA6

By a process similar to the one used in the preparation of molecule AA2 applied to molecule A12 (43 g, 70.3 mmol), the residue obtained after concentration under a vacuum is triturated in acetonitrile. The suspension is filtered, and the solid is washed with acetonitrile then with acetone. After drying under vacuum, a white solid of molecule AA6 in the form of a hydrochloride salt is obtained.

Yield: 31.2 g (81%)

NMR ¹H (DMSO-d₆, ppm): 0.85 (3H); 1.23 (28H); 1.45 (2H); 1.70-2.05 (4H); 2.13 (1H); 2.24 (1H); 2.95 (2H); 3.10-3.25 (211); 3.30-3.65 (10H); 4.20-4.45 (1H); 7.85-8.25 (4H).

LC/MS (ESI): 512.4; (calculated ([M+H]⁺): 512.8).

### EXAMPLE AA7: MOLECULE AA7

### Molecule A13: Product Obtained by Reaction Between Arachidonic Acid and L-Proline.

By a process similar to that used in the preparation of A5 molecule applied to arachidic acid (15.51 g, 49.63 mmol) and L-proline (6 g, 52.11 mmol) using DIPEA in place of TEA, a white solid is obtained after purification by chromatographic column on silica gel (cyclohexane, ethyl acetate, acetic acid).

Yield: 12.9 g (63%)

NMR ¹H (CDCl₃, ppm): 0.88 (3H); 1.28 (34H); 1.66 (2H); 1.95-2.15 (2H); 2.34 (2H); 2.45 (1H); 3.47 (1H); 3.56 (1H); 4.60 (1H).

LC/MS (ESI): 410.4; (calculated ([M+H]⁺): 410.6).

### Molecule A14: Product Obtained by Reaction Between Molecule A13 and Boc-1-Amino-4,7,10-Trioxa-13-Tridecane Amine.

By a process similar to that used in the preparation of molecule A12 applied to molecule A13 (10.96 g, 26.75 mmol) and Boc-1-amino-4,7,10-trioxa-13-tridecane (10.29 g, 32.11 mmol), a solid is obtained after purification by chromatographic column on silica gel (cyclohexane, ethyl acetate, methanol).

Yield: 14.2 g (75%)

NMR ¹H (CDCl₃, ppm): 0.88 (3H); 1.24 (32H); 1.43 (9H); 1.61 (2H); 1.80 (1H); 1.96 (1H); 2.10-2.45 (4H); 3.20-3.75 (18H); 4.30 (0.20H); 4.55 (0.80H); 5.03 (1H); 6.75 (0.20H); 7.20 (0.80H).

LC/MS (ESI): 712.8; (calculated ([M+H]⁺): 713.1).

### Molecule AA7

After a process similar to the one used for the preparation of molecule AA2 applied to molecule A14 (14.25 g, 20.01 mmol), the residue obtained after concentration under a vacuum of the reaction medium is dissolved in methanol and evaporated under reduced pressure, the operation being repeated 4 times to yield a white solid of molecule AA7 in the form of a hydrochloride salt.

Yield: 12.7 g (98%)

NMR ¹H (DMSO-d₆, ppm): 0.85 (3H); 1.23 (32H); 1.45 (2H); 1.64 (2H); 1.70-2.05 (6H); 2.10-2.30 (2H); 2.82 (2H); 3.08 (2H); 3.30-3.60 (15H); 4.15-4.30 (1H); 7.73-8.13 (4H).

LC/MS (ESI): 612.7; (calculated ([M+H]⁺): 612.9).

### EXAMPLE AA8: MOLECULE AA8

### Molecule A15: Product Obtained by the Reaction Between L-Leucine and Palmitoyl Chloride.

By a process similar to the one used in the preparation of molecule A1 applied to L-leucine (15.0 g, 114.4 mmol) and to palmitoyl chloride (34.5 g, 125 mmol), a white solid is obtained by trituration in diisopropyl ether.

Yield: 13.0 g (31%)

NMR ¹H (CDCl₃, ppm): 0.88 (3H); 0.96 (6H); 1.16-1.35 (24H); 1.55-1.77 (5H); 2.23 (2H); 4.55-4.60 (1H); 5.88 (1H).

### Molecule A16: Product Obtained by the Reaction Between Molecule a 15 and the Methyl Ester of L-Proline

By a process similar to that used for the preparation of molecule A2 applied to molecule A15 (6.00 g, 16.2 mmol) and the methyl ester of L-proline (3.23 g, 19.5 mmol), a colorless oil is obtained after purification by flash chromatography (methanol, dichloromethane).

Yield: 5.8 g (74%)

NMR ¹H (CDCl₃, ppm): 0.83-1.00 (9H); 1.18-1.32 (24H); 1.40-1.73 (5H); 1.84-2.33 (6H); 3.47-3.89 (2H); 3.70 (1.14H); 3.71 (1.21H); 3.74 (0.53H); 3.76 (0.12H); 4.40-4.56 (1H); 4.63-4.67 (0.04H); 4.84 (0.38); 4.90 (0.40); 5.06 (0.18); 5.99 (0.18H); 6.08-6.21 (0.82).

LC/MS (ESI): 481.6; (calculated ([M+H]⁺): 481.4).

### Molecule A17: Product Obtained by the Saponification of the Methyl Ester of Molecule A16.

To a solution of molecule A16 (5.8 g, 12.06 mmol) in 30 mL of methanol is added 1N sodium hydroxide (13.5 mL, 13.5 mmol). After stirring for 20 h at room temperature, the solution is diluted with water, then acidified with 20 mL of 1N hydrochloric acid at 0° C. The precipitate is filtered, then rinsed with water (50 ml) before being solubilized in 50 ml of dichloromethane. The organic phase is dried over Na₂SO₄, filtered, then concentrated under reduced pressure to yield a colorless oil.

Yield: 4.5 g (80%)

NMR ¹H (CDCl₃, ppm): 0.85-0.99 (9H); 1.14-1.41 (24H); 1.43-1.72 (5H); 1.87-2.47 (7H); 3.48-3.55 (0.6H); 3.56-3.62 (0.4H); 3.83-3.90 (0.4H); 3.90-3.96 (0.6H); 4.52-4.56 (0.6H); 4.56-4.59 (0.4H); 4.80-4.86 (0.4H); 4.86-4.91 (0.6H); 6.05 (0.4H); 6.11 (0.61H).

LC/MS (ESI): 467.6; (calculated ([M+H]+): 467.4).

### Molecule A18: Product Obtained by Reaction Between N-Boc-Ethylenediamine and Molecule A17.

By a process similar to that used for the preparation of molecule A2 applied to molecule A17 (4.5 g, 9.64 mmol) and BocEDA (1.70 g, 10.61 mmol), a colorless oil is obtained after purification by flash chromatography (methanol, dichloromethane).

Yield: 2.0 g (34%)

NMR ¹H (CDCl₃, ppm): 0.83-0.99 (9H); 1.19-1.32 (24H); 1.44 (9H); 1.48-2.37 (14H); 3.09-3.99 (4H); 4.28-5.01 (2H); 5.64-6.04 (1H); 6.87-7.06 (1H).

LC/MS (ESI): 609.7; (calculated ([M+H]⁺): 609.5).

### Molecule AA8

By a process similar to the one used in the preparation of molecule AA1 applied to molecule A18 (2 g, 3.28 mmol), a white solid of molecule AA8 in the form of a hydrochloride salt is obtained after purification by flash chromatography (methanol, dichloromethane).

Yield: 1.5 g (90%)

NMR ¹H (CDCl₃, ppm): 0.83-1.00 (9H); 1.18-1.32 (24H); 1.37-1.77 (5H); 1.93-2.41 (6H); 3.07-3.97 (6H); 4.44-4.77 (2H); 7.66-8.21 (2H).

LC/MS (ESI); 509.6; (calculated ([M+H]⁺): 509.4).

### EXAMPLE AA9: MOLECULE AA9

### Molecule A19: Product Obtained by the Reaction Between Lauric Acid and L-Phenylalanine.

By a process similar to that used for the preparation of A5 molecule applied to lauric acid (8.10 g, 40.45 mmol) and L-phenylalanine (7 g, 42.38 mmol), a white solid is obtained.

Yield: 12.7 g (98%)

NMR ¹H (DMSO-d₆, ppm): 0.86 (3H); 1.10-1.30 (16H); 1.36 (2H); 2.02 (2H); 2.82 (1H); 3.05 (1); 4.42 (1H); 7.15-7.30 (5H); 8.05 (1H); 12.61 (1H).

LC/MS (ESI): 348.2; (calculated ([M+H]⁴): 348.5).

### Molecule A20; Product Obtained by the Reaction Between Molecule A19 and L-Proline Methyl Ester Hydrochloride Salt.

By a process similar to that used in the preparation of molecule A6 applied to molecule A19 (9.98 g, 28.72 mmol) and to L-proline methyl ester hydrochloride salt (5.23 g, 31.59 mmol), a colorless oil is obtained after purification by chromatographic column on silica gel (cyclohexane, ethyl acetate).

Yield: 5.75 g (44%)

NMR ¹H (CDCl₃, ppm): 0.88 (3H); 1.10-1.30 (16H); 1.50-1.75 (311); 1.80-2.02 (3H); 2.17 (2H); 2.65 (0.5H); 2.95 (1H); 3.05-3.20 (1.5H); 3.50-3.65 (1H); 3.75 (3H); 4.29 (0.5H); 4.46 (0.5H); 4.70 (0.1H); 4.95 (0.9H); 6.20-6.30 (1H); 7.15-7.30 (5H).

LC/MS (ESI): 459.2; (calculated ([M+H]⁴): 459.6).

### Molecule A21: Product Obtained by Saponification of Molecule A20.

Lithium hydroxide (LiOH) (600.49 mg, 25.07 mmol) is added to a solution of molecule A20 (5.75 g, 12.54 mmol) in a THF/methanol/water mixture (40/40/40 mL) at 0° C., then the mixture is stirred at room temperature for 20 hours. After evaporation of the organic solvents under vacuum, the aqueous solution is diluted in water, acidified with an 1N HCl aqueous solution to a pH of 1. The product is then extracted with ethyl acetate. The combined organic phases are washed with a saturated aqueous NaCl solution, dried over Na₂SO₄, filtered and concentrated under reduced pressure to yield a colorless oil.

Yield: 5.7 g (quantitative)

NMR ¹H (CDCl₃, ppm): 0.88 (3H); 1.10-1.30 (16H); 1.50-1.80 (3H); 1.67-2.02 (2H); 2.20 (2H); 2.25 (0.4H); 2.60 (0.6H); 2.85-3.10 (2.6H); 3.55-3.65 (1.4H); 4.35 (0.6H); 4.55 (0.4H); 4.94 (1H); 6.28 (0.4H); 6.38 (0.6H); 7.20-7.30 (5H).

LC/MS (ESI): 445.2; (calculated ([M+H]⁺): 445.6).

### Molecule A22: Product Obtained by Reaction Between N-Boc-Ethylenediamine and Molecule A21.

By a process similar to the one used in the preparation of molecule A6 applied to molecule A21 (5.67 g, 12.75 mmol) and BocEDA (2.25 g, 14.03 mmol), a colorless oil is obtained after purification by chromatography column on silica gel (dichloromethane, methanol).

Yield: 5.7 g (76%)

NMR ¹H (CDCl₃, ppm): 0.88 (3H); 1.25 (16H); 1.43 (9H); 1.58 (2.6H); 1.75-1.95 (1.4H); 2.15-2.30 (3H); 2.64 (0.5H); 2.95-3.10 (2.5H); 3.20-3.40 (4H); 3.45 (0.5H); 3.55 (0.2H); 3.66 (1H); 4.44 (1H); 4.50 (0.2H); 4.60 (0.6H); 4.99 (0.7H); 5.54 (0.5H); 5.95 (0.2H); 6.17 (1H); 6.60 (0.5H); 7.07 (0.5H); 7.20-7.40 (5H).

LC/MS (ESI): 587.4; (calculated ([M+H]⁺): 587.8).

### Molecule AA9

Following a process similar to that used for the preparation of molecule AA2 applied to molecule A22 (5.66 g, 9.65 mmol), the residue obtained after concentration of the reaction medium under vacuum is dissolved in methanol and evaporated under reduced pressure; the operation being repeated 4 times to produce a white foam of molecule AA9 in the form of hydrochloride salt.

Yield: 4.9 g (97%)

NMR ¹H (DMSO-d₆, 120° C., ppm): 0.89 (3H); 1.26 (16H); 1.43 (2H); 1.68 (0.6H); 1.75-2.00 (3H); 2.05-2.25 (2.4H); 2.82-3.05 (5H); 3.38 (2H); 3.50-3.70 (1.4H); 4.25 (0.6H); 4.63 (0.4H); 4.77 (0.6H); 7.25-7.50 (5H); 7.55-8.20 (4H).

LC/MS (ESI): 487.4; (calculated ([M+H]⁺): 487.7).

### EXAMPLE AA10: MOLECULE AA10

### Molecule A23: Product Obtained by the Reaction Between Molecule B7 and N-Boc-Ethylenediamine

HOBt (8.94 g, 58.37 mmol), then BocEDA (112.20 g, 700.00 mmol) in solution in DCM (150 mL) at 0° C. are successively added to a solution of molecule B7 (190.00 g, 583.73 mmol) in DCM (2.9 L). EDC (123.10 g, 642.00 mmol) is then added, then the mixture is stirred for 17 hours between 0° C. and at room temperature. The reaction mixture is then washed with a saturated aqueous NaHCO₃ (2×1.5 L) solution, an 1N HCl aqueous solution (2×1.5 L), then a saturated aqueous NaCl solution (1.5 L), dried over Na₂SO₄, filtered and concentrated under reduced pressure. A white solid is obtained after recrystallization in acetonitrile.

Yield: 256.50 g (93%)

NMR ¹H (CDCl₃, ppm): 0.88 (3H); 1.16-1.38 (20H); 1.44 (9H); 1.56-1.71 (2H); 1.78-2.45 (6H); 3.11-3.72 (6H); 4.30 (0.1H); 4.51 (0.9H); 4.87 (0.11H); 5.04 (0.9H); 6.87 (0.1H); 7.23 (0.9H). LC/MS (ESI): 468.0; (calculated ([M+H]⁺): 468.4).

### Molecule AA10

Following a process similar to that used in the preparation of molecule AA1 applied to molecule A23 (256.50 g, 548.43 mmol), a white solid of molecule AA10 in the form of a hydrochloride salt is obtained by trituration in pentane (1.6 L) and drying under reduced pressure at 40° C.

Yield: 220.00 g (99%)

NMR ¹H (MeOD-d4, ppm): 0.90 (3H); 1.21-1.43 (20H); 1.54-1.66 (2H); 1.85-2.28 (4H); 2.39 (2H); 3.00-3.17 (2H); 3.30-3.40 (1H); 3.43-3.71 (3H); 4.29 (0.94H); 4.48 (0.06H).

LC/MS (ESI): 368.2; (calculated ([M+H]⁺): 368.3).

### EXAMPLE AA11: MOLECULE AA11

### Molecule A24: Product Obtained by Reaction Between Molecule B7 and Boc-1-Amino-4,7,10-Trioxa-13-Tridecane Amine.

By a process similar to that used in the preparation of molecule A23 applied to molecule B7 (24.00 g, 73.73 mmol) and Boc-1-amino-4,7,10-trioxa-13-tridecane amine (28.35 g, 88.48 mmol), an orange oil of molecule A24 is obtained.

Yield: 44.50 g (96%)

1H NMR (CDCl₃, ppm): 0.87 (3H); 1.08-1.56 (20H); 1.43 (9H); 1.58-1.67 (2H); 1.70-2.00 (6H); 2.04-2.41 (4H); 3.16-3.77 (18H); 4.26-4.29 (0.2H); 4.50-4.54 (0.8H); 4.68-5.10 (1H); 6.74 (0.2H); 7.19 (0.8H).

LC/MS (ESI): 628.4; (calculated ([M+H]): 628.5).

### Molecule AA11

Following a process similar to that used in the preparation of AA1 molecule applied to A24 molecule (43.40 g, 69.12 mmol), a white solid of molecule AA11 in the form of hydrochloride salt is obtained after trituration (3 times) in diethyl ether, solubilization of the residue in water and lyophilization.

Yield: 38.70 g (98%)

1H NMR (DMSO, ppm): 0.85 (3H); 1.07-1.38 (20H); 1.41-1.52 (2H); 1.55-1.66 (2H); 1.70-2.02 (6H); 2.08-2.30 (2H); 2.78-2.87 (2H); 3.00-3.16 (2H); 3.29-3.66 (14H); 4.16-4.22 (0.65 H); 4.25-4.30 (0.35H); 7.74 (0.65H); 7.86 (3H); 8.10 (0.35H).

LC/MS (ESI): 528.4; (calculated ([M+H]⁺): 528.4).

### EXAMPLE AA12: MOLECULE AA12

### Molecule A25: Product Obtained by the Reaction Between Molecule B4 and N-Boc-Ethylenediamine.

By a process similar to that used in the preparation of molecule A23 applied to molecule B4 (12.00 g, 40.35 mmol) and to Boc-ethylenediamine (7.76 g, 48.42 mmol), a colorless oil is obtained and used without further purification.

Yield: 17.40 g (94%)

1H NMR (CDCl₃, ppm): 0.86 (3H); 1.11-1.68 (18H); 1.41 (9H); 1.80-2.38 (6H); 3.06-3.35 (4H); 3.37-3.49 (1H); 3.51-3.73 (1H); 4.26-4.31 (0.1H); 4.45-4.52 (0.9H); 4.91-5.19 (1H); 6.97 (0.1H); 7.23 (0.9H). LC/MS (ESI): 440.4 (calculated ([M+H]J): 440.3).

### Molecule AA12

Following a process similar to that used in the preparation of molecule AA1 applied to molecule A25 (8.85 g, 20.13 mmol), a white solid of molecule AA12 is obtained after basic washing, concentration under reduced pressure, then recrystallization in acetonitrile.

Yield: 6.53 g (96%)

1H NMR (DMSO, ppm): 0.85 (3H); 1.07-1.56 (20H); 1.68-2.03 (4H); 2.09-2.29 (2H); 2.50-2.58 (2H); 2.96-3.11 (2H); 3.21-3.59 (2H); 4.17-4.21 (0.65H); 4.25-4.29 (0.35H); 7.68 (0.65H); 8.00 (0.35H)

LC/MS (ESI): 340.3; (calculated ([M+H]⁺): 340.3).

### EXAMPLE AA13: MOLECULE AA13

### Molecule A26: Product Obtained by Coupling Between Molecule B1 and the N-Boc-Ethylenediamine.

By a process similar to that used in the preparation of molecule A23 applied to molecule B1 (30.00 g, 111.36 mmol) and BocEDA (21.41 g, 133.64 mmol), a white solid is obtained after recrystallization in acetonitrile.

Yield: 34.90 g (76%)

1H NMR (CDCl₃, ppm): 0.88 (3H); 1.10-1.70 (14H); 1.43 (9H); 1.80-1.91 (1H); 1.92-2.01 (1H); 2.04-2.42 (4H); 3.13-3.70 (6H); 4.27-4.31 (0.15H); 4.47-4.53 (0.85H); 4.83 (0.15H); 5.02 (0.85H); 6.85 (0.15H); 7.21 (0.85H).

LC/MS (ESI): 412.2; (calculated ([M+H]⁺): 412.3).

### Molecule AA13

Following a process similar to that used in the preparation of molecule AA1 applied to molecule A26 (34.90 g, 84.79 mmol), a white solid of molecule AA13 in the form of hydrochloride salt is obtained after solubilization in a DCM/acetonitrile mixture and concentration under reduced pressure.

Yield: 29.50 g (99%)

1H NMR (DMSO, ppm): 0.85 (3H); 1.07-1.61 (14H); 1.70-2.06 (4H); 2.10-2.35 (2H); 2.76-2.87 (2H); 3.24-3.47 (3.25H); 3.56-3.64 (0.75H); 4.13-4.19 (0.75H); 4.31-4.36 (0.25H); 8.05-8.36 (3.75H); 8.50 (0.25H).

LC/MS (ESI): 312.2; (calculated ([M+H]⁺): 312.3).

### EXAMPLE AA14: MOLECULE AA14

### Molecule A27: Product Obtained by Hydrogenation of Phytol.

Platinum oxide (PtO₂, 1.15 g, 6.61 mmol) is added to a solution of phytol (30.00 g, 101.20 mmol) in THF (450 mL) under argon and the medium is placed under 1 bar of dihydrogen, then stirred for 4 hours at room temperature. After filtration through celite by rinsing with THF, a black oil of molecule A27 is obtained after concentration at reduced pressure.

Yield: 29.00 g (96%)

NMR ¹H (CDCl₃, ppm): 0.84 (6H); 0.86 (6H); 0.89 (3H); 1.00-1.46 (22H); 1.46-1.68 (3H); 3.61-3.73 (2H).

### Molecule A28: Product Obtained by Oxidation of Molecule A27

Tetrabutylammonium bromide (16.90 g, 52.45 mmol) acetic acid (150 mL, 2.62 mol) followed by KMnO₄ (46.05 g, 291.40 mmol) are successively added to a solution of molecule A27 (29.0 g, 97.13 mmol) in a dichloroethane/water mixture (485 mL/388 mL) in small portions keeping the temperature between 16 and 19° C. The reaction medium is then stirred for 4 hours 30 minutes at reflux, cooled to 10° C., then acidified until pH 1 with a solution of 6 N HCl (20 mL). Na₂SO₃ (53.90 g) is then added gradually while maintaining the temperature at 10° C., and the mixture is stirred until complete decolorization. Water (200 mL) is added, the phases are separated, and the aqueous phase is extracted with DCM (2×400 mL). The combined organic phases are washed with 10% HCl aqueous solution (20 mmL), water (2×200 mL), a saturated aqueous solution of NaCl (200 mL), dried over Na₂SO₄, filtered and concentrated under reduced pressure. A yellow oil of molecule A28 is obtained after purification by flash chromatography (eluent: cyclohexane, AcOEt).

Yield: 28.70 g (94%)

NMR ¹H (CDCl₃, ppm): 0.84 (6H); 0.86 (6H); 0.97 (3H); 1.00-1.41 (20H); 1.52 (1H); 1.96 (1H); 2.14 (1H); 2.35 (1H); 11.31 (1H).

LC/MS (ESI): 311.1 (calculated ([M−H]⁻): 311.3).

### Molecule A29: Product Obtained by Coupling Between Molecule A28 and Methyl L-Prolinate.

By a process similar to that used in the preparation of molecule A2 applied to molecule A28 (18.00 g, 57.59 mmol) and methyl L-prolinate hydrochloride (14.31 g, 86.39 mmol) a yellow oil of molecule A29 is obtained after washing the organic phase with a NaHCO₃ (2×150 mL) saturated aqueous solution, a 10% aqueous solution of HCl (2×150 mL), a saturated aqueous solution of NaCl (2×150 mL), then drying on Na₂SO₄, filtration and concentration under reduced pressure.

Yield: 23.20 g (95%)

NMR ¹H (DMSO-d₆, ppm): 0.78-0.89 (15H); 0.97-1.43 (20H); 1.43-1.56 (1H); 1.70-1.96 (4H); 1.96-2.32 (3H); 3.33-3.56 (2H); 3.59 (0.6H); 3.67 (2.4H); 4.27 (0.8H); 4.57 (0.2H). LC/MS (ESI): 424.4 (calculated ([M+H]⁺): 424.4).

### Molecule A30: Product Obtained by the Saponification of Molecule A29.

By a process similar to the one used in the preparation of molecule A21 applied to molecule A29 (21.05 g, 49.68 mmol), a yellow oil of molecule A30 is obtained.

Yield: 20.40 g (99%)

NMR ¹H (DMSO-d, ppm): 0.77-0.91 (15H); 0.97-1.43 (20H); 1.43-1.56 (1H); 1.67-1.96 (4H); 1.96-2.29 (3H); 3.26-3.56 (2H); 4.20 (0.8H); 4.41 (0.2H).

LC/MS (ESI): 410.3 (calculated ([M+H]⁺): 410.4).

### Molecule A31: Product Obtained by Reaction Between Molecule A30 and Boc-1-Amino-4,7,10-Trioxa-13-Tridecane Amine.

By a process similar to that used in the preparation of molecule A23 applied to molecule A30 (8.95 g, 21.85 mmol) and Boc-1-amino-4,7,10-trioxa-13-tridecane amine (8.40 g, 26.21 mmol), a colorless oil of molecule A31 is obtained after purification by flash chromatography (eluent: DCM, AcOEt, methanol).

Yield: 10.08 g (65%)

NMR ¹H (DMSO-d₆, ppm): 0.78-0.89 (15H); 0.97-1.43 (29H); 1.43-1.55 (1H); 1.55-1.66 (411); 1.71-2.30 (7H); 2.95 (2H); 3.00-3.19 (2H); 3.34-3.58 (14H); 4.17-4.29 (1H); 6.30-6.79 (1H); 7.67 (0.65H); 8.00 (0.35H).

LC/MS (ESI): 712.6 (calculated ([M+H]⁺): 712.6).

### Molecule AA14

Following a process similar to that used in the preparation of AA1 molecule applied to A31 molecule (10.08 g, 14.16 mmol), the residue obtained after concentration under reduced pressure is solubilized in DCM (200 mL). The organic phase is washed with an aqueous solution of 2N NaOH (2×100 mL), dried over Na₂SO₄, filtered and concentrated under reduced pressure. A colorless oil of AA14 molecule in neutral amine form is obtained.

Yield: 8.23 g (95%)

NMR ¹H (DMSO-d₆, ppm): 0.78-0.89 (15H); 0.97-1.43 (20H); 1.43-1.69 (6H); 1.69-2.30 (8H); 2.56 (2H); 2.99-3.19 (2H); 3.31-3.58 (14H); 4.15-4.29 (1H); 7.70 (0.65H); 8.04 (0.35H).

LC/MS (ESI): 612.5 (calculated ([M+H]⁺): 612.5).

### EXAMPLE AA15: MOLECULE AA15

AA15 molecule is obtained by the conventional method of solid phase peptide synthesis (SPPS) on 2-chlorotrityl resin.

DIPEA (8.64 mL, 49.60 mmol) is added to a solution of 4,7,10-trioxa-1,13-tridecanediamine (TOTA, 10.87 mL, 49.60 mmol) in DCM (50 mL). This solution is then poured onto 2-chlorotrityl resin (4.00 g, 1.24 mmol/g) previously washed with DCM in a reactor adapted to SPPS. After stirring for 2 hours at room temperature, methanol (0.8 mL/g, 3.2 mL) is added and the medium is stirred for 15 minutes. The resin is filtered, washed successively with DCM (3×50 mL), DMF (2×50 mL), DCM (2×50 mL), isopropanol (1×50 mL) and DCM (3×50 mL). Protected amino acids N-Fmoc-L-glycine and N-Fmoc-L-proline, then palmitic acid (3 equivalents) are coupled successively using 1-[bis (dimethylamino) methylene]-1H-1,2,3-triazolo [4.5-b] pyridinium 3-oxide hexafluorophosphate (HATU, 3 equivalents) as coupling agent in the presence of DIPEA (6 equivalents) in a DCM/DMF 1 mixture: 1. A solution of 20% piperidine in DMF is used for the cleavage steps of the Fmoc protecting group. The resin is washed with DCM, DMF and isopropanol after each coupling and deprotection step. Cleavage of the resin product is carried out using a TFA/DCM 1 mixture: 1. The solvents are evaporated under reduced pressure, the residue is solubilized in DCM (50 mL) and the organic phase is washed with 1N aqueous solution of NaOH (1×50 mL), then a saturated solution of NaCl (2×50 mL). After drying on Na₂SO₄, the organic phase is filtered, concentrated under reduced pressure and the residue is purified by chromatography on silica gel (dichloromethane, methanol, NH₄OH).

Yield: 1.65 g (54% overall over 7 steps).

NMR ¹H (CDCl₃, ppm): 0.88 (3H); 1.18-2.39 (38H); 2.79 (2H); 3.23-3.44 (2H); 3.47-3.69 (14H); 3.76 (0.92H); 3.82 (0.08H); 3.98 (0.08H); 4.03 (0.92H); 4.34 (0.08H); 4.39 (0.92H); 7.00-7.40 (2H).

LC/MS (ESI): 613.7; (calculated ([M+H]+): 613.5).

### EXAMPLE AA16: MOLECULE AA16

By a SPPS process similar to that used in the preparation of molecule AA15 and using the N-Fmoc-L-phenylalanine (3 equivalents) instead of N-Fmoc-L-glycine, molecule AA16 is obtained in the form of a yellow oil.

Yield: 14.07 g (69%)

NMR ¹H (CDCl₃, ppm): 0.88 (3H); 1.19-1.34 (24H); 1.41-1.61 (2H); 1.68-2.28 (12H); 2.84 (2H); 3.14 (2H); 3.23-3.67 (16H); 4.19-4.25 (0.1H); 4.38-4.45 (0.9H); 4.59-4.69 (1H); 6.86 (1H); 7.03 (1H); 7.12-7.30 (5H).

LC/MS (ESI): 703.5; (calculated ([M+H]⁺): 703.5).

### EXAMPLE AA17: MOLECULE AA17

Molecule AA17 is obtained in the form of a white solid through a SPPS process similar to that used in the preparation of AA15 molecule and using EDA (30.48 mL, 0.456 mol) instead of TOTA.

Yield: 9.19 g (89%)

NMR ¹H (MeOD-d4, ppm): 0.90 (3H); 1.22-1.43 (24H); 1.55-1.67 (2H); 1.91-2.04 (2H); 2.04-2.15 (1H); 2.17-2.29 (1H); 2.39 (2H); 2.69-2.82 (2H); 3.25-3.36 (2H); 3.58-3.70 (2H); 3.70-3.97 (2H); 4.25-4.34 (0.9H); 4.44-4.50 (0.1H).

LC/MS (ESI): 453.3; (calculated ([M+H]⁺): 453.4).

### EXAMPLE AA18: MOLECULE AA18

By a SPPS process similar to that used in the preparation of molecule AA15 and successively using ethylenediamine (20 equivalents), N-Fmoc-L-phenylalanine (1.5 equivalents) and molecule B7 (1.5 equivalents), molecule AA18 is obtained in the form of a white solid.

Yield: 12.76 g (85%)

NMR ¹H (MeOD-d4, ppm): 0.90 (3H); 1.14-1.65 (22H); 1.73-2.41 (6H); 2.56-2.70 (2H); 2.91-3.26 (4H); 3.41-3.63 (2H); 4.30 (0.8H); 4.39 (0.2H); 4.53 (0.8H); 4.61 (0.2H); 7.19-7.31 (5H).

LC/MS (ESI): 515.4; (calculated ([M+H]⁺): 515.4).

### AB: Synthesis of Co-Polyamino Acids

Statistical co-polyamino acids according to formula VII or VIIa

TABLE 1b
list of co-polyamino acids synthesized according to the invention

| No | CO-POLYAMINOACIDES BEARING CARBOXYLATE LOADS AND HYDROPHOBIC RADICALS |
|---|---|
| AB1 | 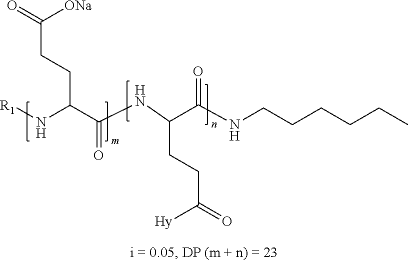 |
| | 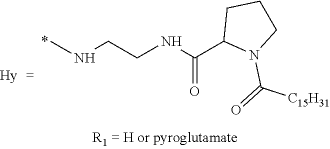 |
| AB2 | 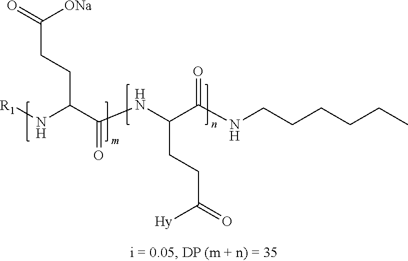 |
| | 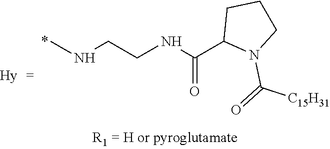 |
| AB3 | 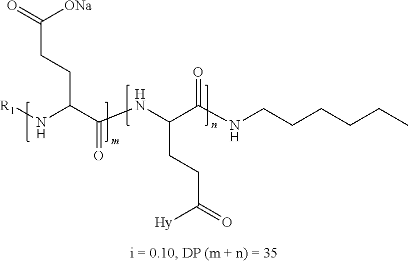 |
| | 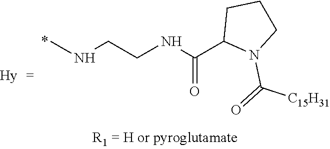 |
| AB4 | 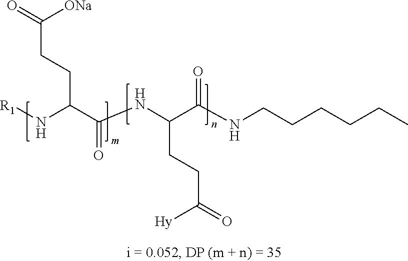 |
| | 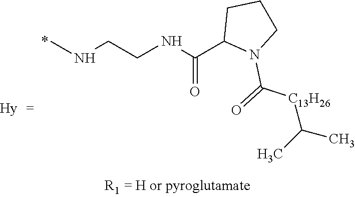 |
| AB5 | 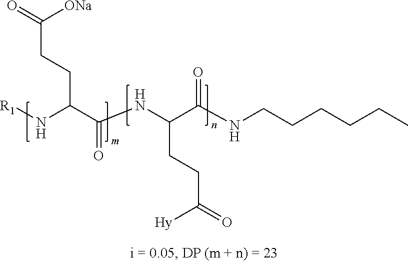 |
| | 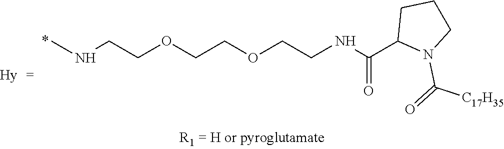 |
| AB6 | 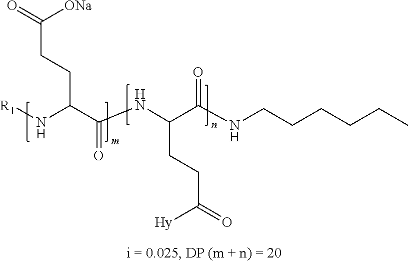 |
| | 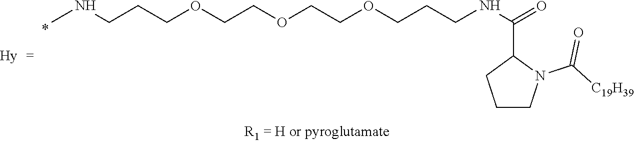 |
| AB7 | 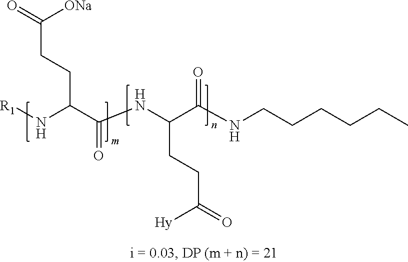 |
| | 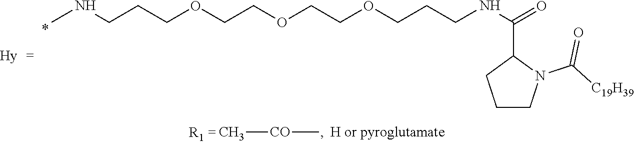 |
| AB8 | 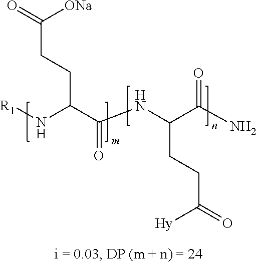 |
| | 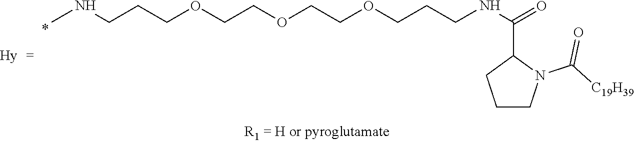 |
| AB9 | 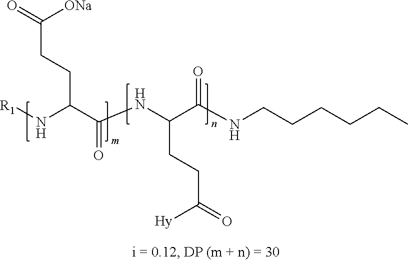 |
| | 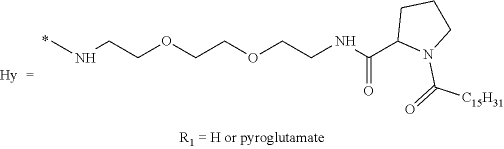 |
| AB10 | 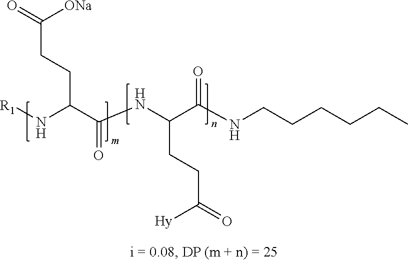 |
| | 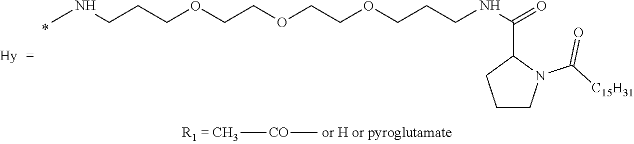 |
| AB11 | 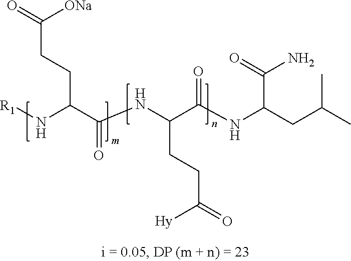 |
| | 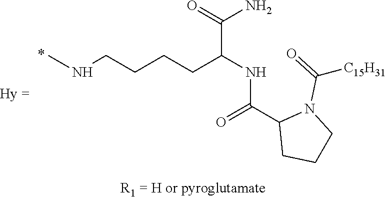 |
| AB12 | 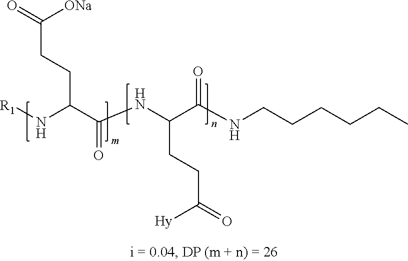 |
| | 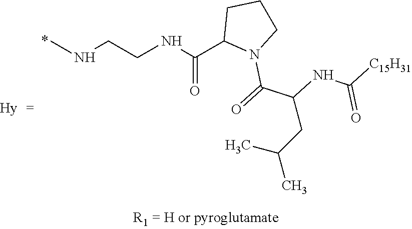 |
| AB13 | 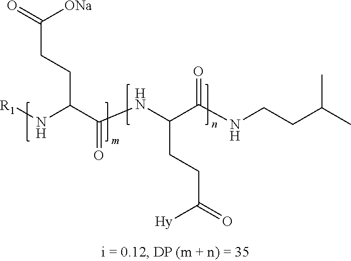 |
| | 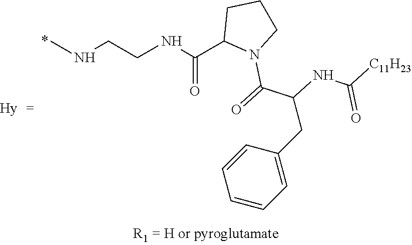 |
| AB21 | 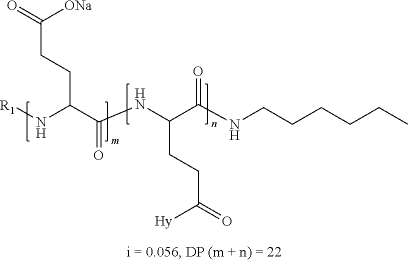 |
| | 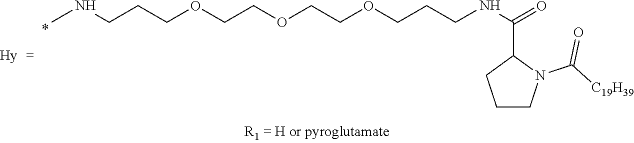 |
| AB22 | 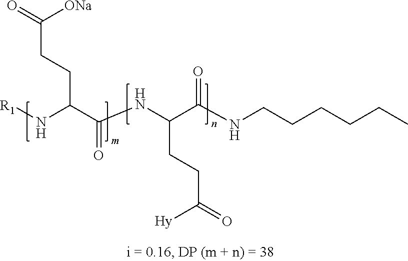 |
| | 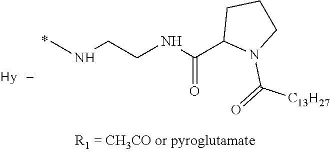 |
| AB23 | 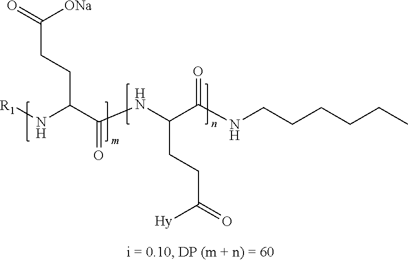 |
| | 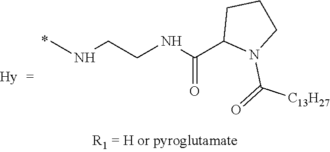 |
| AB24 | 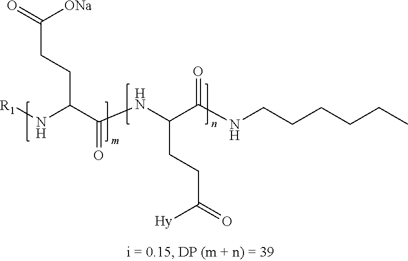 |
| | 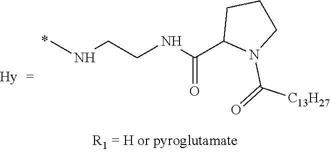 |
| AB25 | 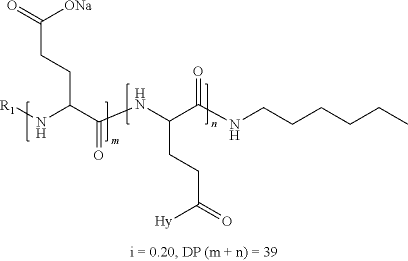 |
| | 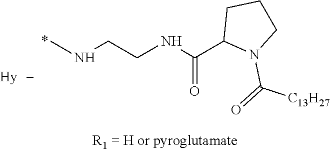 |
| AB26 | 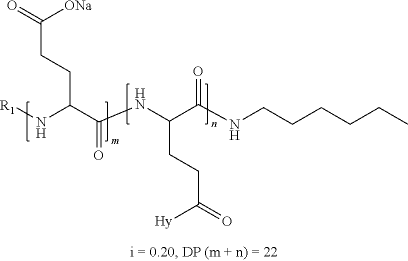 |
| | 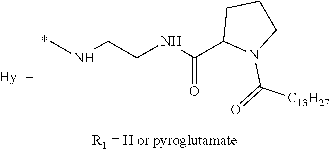 |
| AB27 | 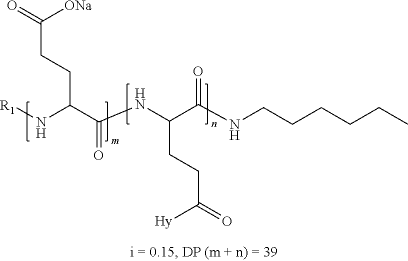 |
| | 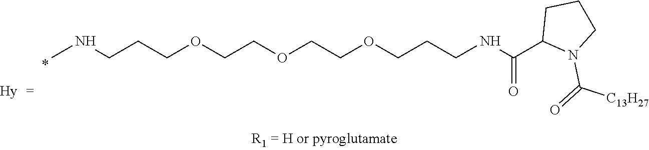 |
| AB28 | 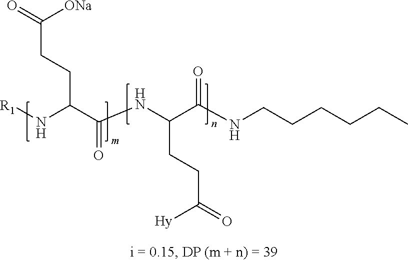 |
| | 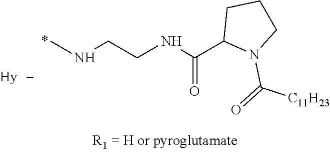 |
| AB29 | 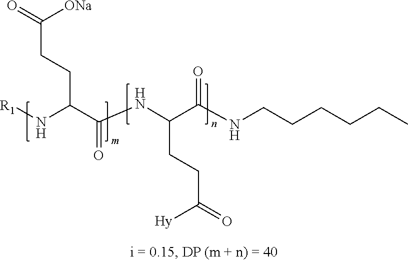 |
| | 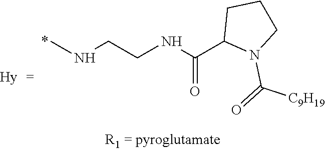 |
| AB30 | 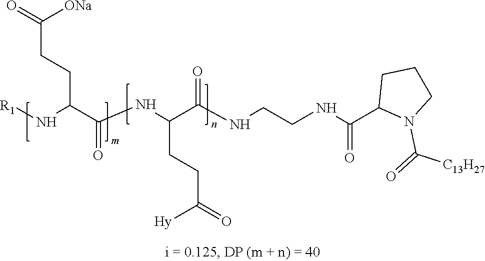 |
| | 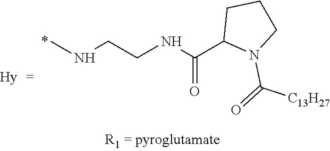 |
| AB31 | 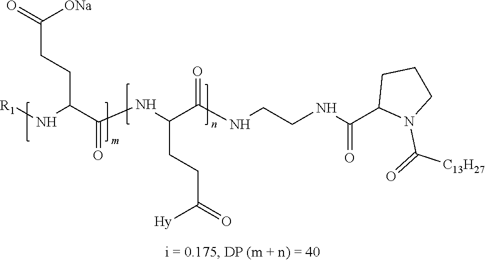 |
| | 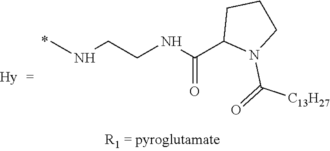 |
| AB32 | 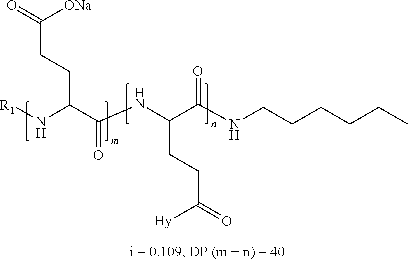 |
| | 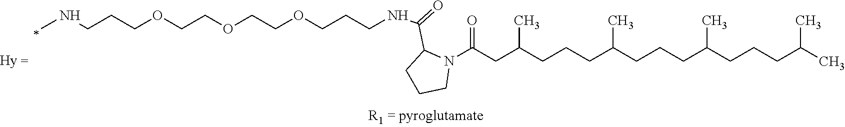 [SMILES: CNCCCOCCOCCOCCCNC(=O)C1CCCN1C(=O)CC(C)CCCC(C)CCCC(C)CCCC(C)C] |
| AB38 | 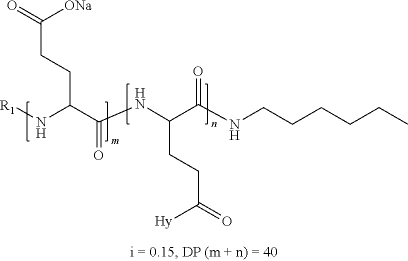 |
| | 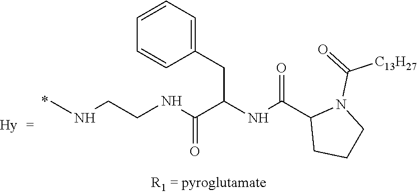 |

Co-polyamino acids according to formula VII or VIIb:

TABLE 1c
List of co-polyarnino acids synthesized according to the invention,

| No | CO-POLYAMINOACIDES BEARING CARBOXYLATE LOADS AND HYDROPHOBIC RADICALS |
|---|---|
| AD14 | 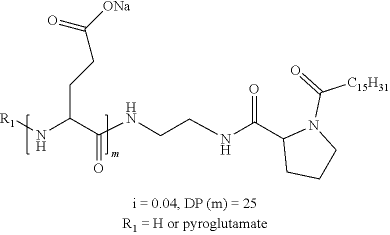 |
| AB15 | 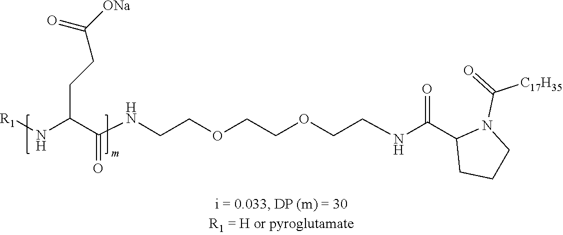 |
| AB16 | 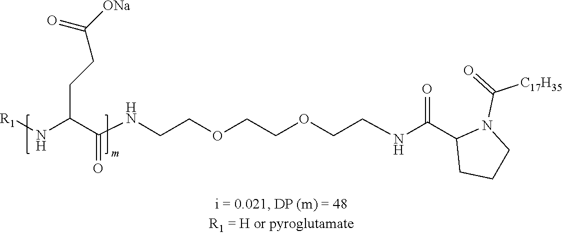 |
| AB17 | 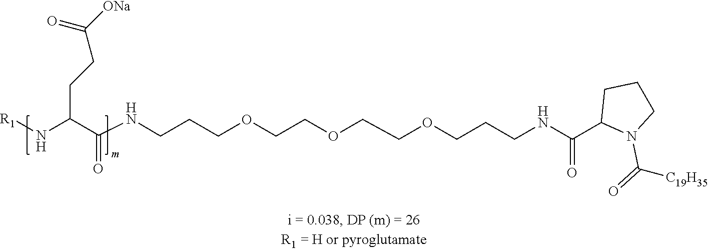 |
| AB18 | 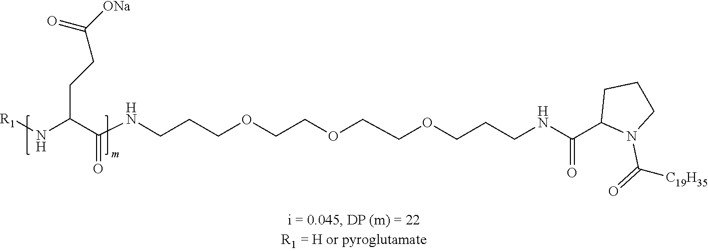 |
| AB19 | 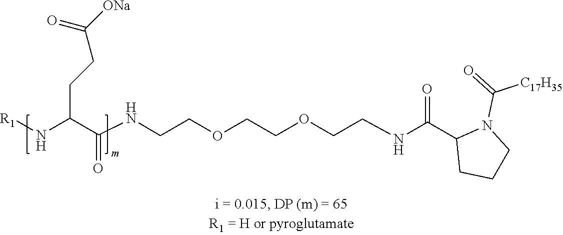 |
| AB20 | 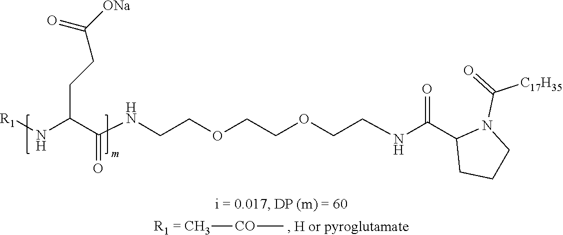 |
| AB33 | 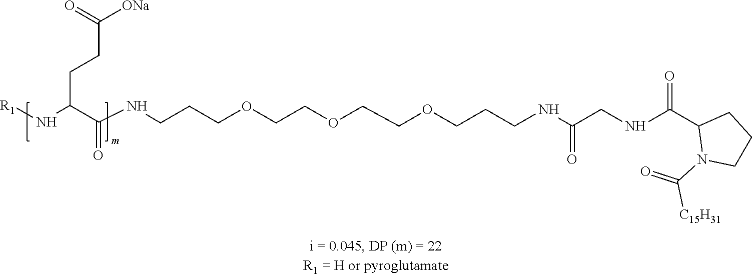 |
| AB34 | 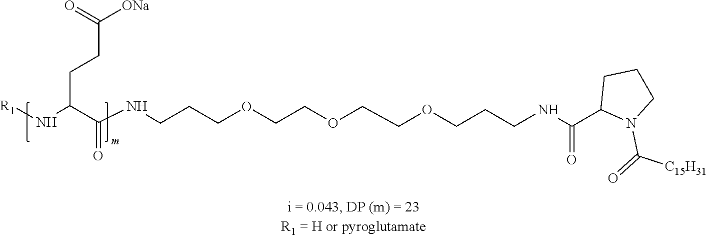 |
| AB35 | 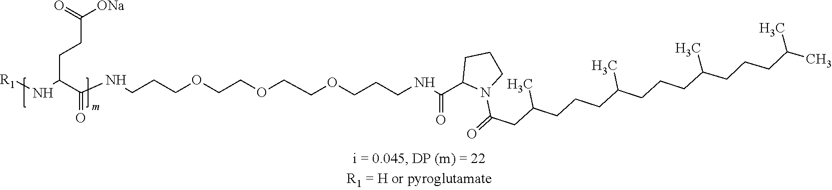 |
| AB36 | 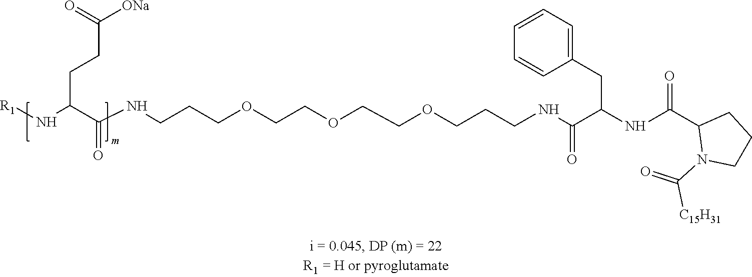 |
| AB37 | 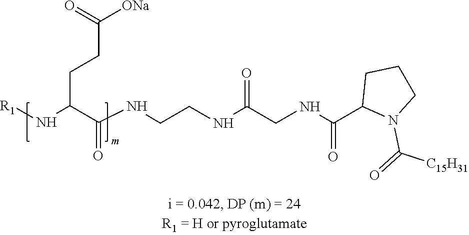 |

### Part AB: Synthesis of Co-Polyamino Acids

### EXAMPLE AB1: CO-POLYAMINO ACID AB1—SODIUM POLY-L-GLUTAMATE MODIFIED BY MOLECULE AA1 AND HAVING A NUMBER AVERAGE MOLECULAR WEIGHT (MN) OF 2900 G/MOL

Co-Polyamino Acid AB1-1: Poly-L-Glutamic Acid of Number-Average Molar Mass (Mn) 3861 g/Mol from the Polymerization of γ-Benzyl-L-Glutamate N-Carboxyanhydride Initiated by Hexylamine

In a previously oven-dried flask is placed under vacuum γ-benzyl-L-glutamate N-carboxyanhydride (89.9 g, 341 mmol) for 30 minutes, then anhydrous DMF (200 mL) is introduced. The mixture is then stirred under argon until complete dissolution, cooled to 4° C., then hexylamine (2.05 mL 15.5 mmol) is quickly introduced. The mixture is stirred at 4° C. and room temperature for 2 days. The reaction medium is then heated at 65° C. for 2 hours, cooled to room temperature, then poured dropwise into diisopropyl ether (3 L) with stirring. The white precipitate is recovered by filtration, washed with diisopropyl ether (2×200 mL), then dried under vacuum at 30° C. to give a poly (gamma-benzyl-L-glutamic acid) (PBLG).

A solution of hydrobromic acid (HBr) at 33% in acetic acid (240 mL, 1.37 mol) is added dropwise—to a solution of PBLG (74.8 g) in trifluoroacetic acid (TFA, 340 mL) at 4° C. The mixture is stirred at room temperature for 2 hours, then poured dropwise onto a 1: 1 (v/v) mixture of diisopropyl ether and water with stirring (4 L). After stirring for 2 hours, the heterogeneous mixture is allowed to stand overnight. The white precipitate is recovered by filtration, washed with a 1:1 (v/v) mixture of diisopropyl ether and water (340 mL), then with water (340 mL).

The obtained solid is solubilized in water (1.5 mL) by adjusting the pH to 7 by adding 10 N aqueous sodium hydroxide solution, then 1N aqueous sodium hydroxide solution. After solubilization, the theoretical concentration is adjusted to 20 g/L theoretical by addition of water to obtain a final volume of 2.1 mL.

The solution is filtered through a 0.45 μm filter, then purified by ultrafiltration against a solution of NaCl 0.9%, then water until the conductimetry of the permeate is less than 50 μS/cm. The solution of co-polyamino acid is then concentrated until a final volume of 1.8 L is obtained.

The aqueous solution is then acidified by adding a 37% hydrochloric acid solution until a pH of 2 is reached. After stirring for 4 hours, the precipitate obtained is filtered, washed with water (2×340 mL), then dried under vacuum at 30° C. to give a poly-L-glutamic acid of number-average molar mass (Mn) 3861 g/mol relative to a standard of polyoxyethylene (PEG).

### Co-Polyamino Acid AB1

Co-polyamino acid AB1-1 (10.0 g) is solubilized in DMF (700 mL) at 30-40° C., then cooled to 0° C. Molecule AA1 in the form of hydrochloride salt (1.64 g, 3.8 mmol) is suspended in DMF (23 mL) and triethylamine (0.39 g, 3.8 mmol) is then added and the mixture is slightly heated while stirring until complete dissolution. N-methylmorpholine (NMM, 7.6 g, 75 mmol) in DMF (14 mL) and ethyl chloroformate (ECF, 8.2 g, 75 mmol) are added to a solution of co-polyamino acid at 0° C. After 10 minutes at 0° C., the solution containing the molecule AA1 is added and the medium maintained at 30° C. for 2 hours. The reaction mixture is poured dropwise over 5.5 L of water containing 15% NaCl weight and HCl (pH 2), and left to stand overnight. The precipitate is recovered by filtration and dried under vacuum for about 30 minutes. The white solid obtained is taken up in water (500 ml) and the pH is adjusted to 7 by slow addition of a 1N aqueous solution of NaOH. After filtration on a 0.45 μm filter, the clear solution obtained is purified by ultrafiltration against 0.9% NaCl solution, then with water, until the conductimetry of the permeate is less than 50 μS/cm. After removal, the solution is filtered through a 0.2 μm filter and stored at 2-8° C.

Dry extract: 24.9 mg/g

A mean degree of polymerization (DP) of 23 is estimated by NMR ¹H in D20 comparing the integration of the signals from the grafted hydrophobe to that of the signals from the main chain.

Based on ¹H NMR: i=0.05

The calculated average molar mass of co-polyamino acid AB1 is calculated on the basis of the molar masses of radicals R₁ and R₂, aspartate and/or glutamate residues (including an amide linkage), hydrophobic radical, DS and DP.

The calculated average molar mass of the co-polyamino acid AB1 is 3945 g/mol.
Aqueous HPLC-SEC (PEG calibrant): Mn=2900 g/mol.

### EXAMPLE AB2: CO-POLYAMINO ACID AB2—SODIUM POLY-L-GLUTAMATE MODIFIED BY MOLECULE AA1 AND HAVING A NUMBER-AVERAGE MOLECULAR WEIGHT (MN) OF 3700 G/MOL

A sodium poly-L-glutamate modified by the molecule AA1 is obtained by a process similar to that used for the preparation of the co-polyamino acid AB1 applied to the hydrochloride salt of molecule AA1 (1.64 g, 3.8 mmol) and a poly-L-glutamic acid of Mn relative 5200 g/mol (10.0 g) obtained by a process similar to that used for the preparation of the co-polyamino acid AB1-1.

Dry extract: 14.1 mg/g
DP (estimated based on RMN ¹H): 35
Based on ¹H NMR: i=0.05
The calculated average molar mass of the co-polyamino acid AB2 is 5972 g/mol.
Aqueous HPLC-SEC (PEG calibrant): Mn=3700 g/mol.

### EXAMPLE AB3: CO-POLYAMINO ACID AB3—SODIUM POLY-L-GLUTAMATE MODIFIED BY MOLECULE AA1 AND HAVING A NUMBER-AVERAGE MOLECULAR WEIGHT (MN) OF 4900 G/MOL

A sodium poly-L-glutamate modified with molecule AA1 is obtained by a process similar to that used in the preparation of the co-polyamino acid AB applied to the hydrochloride salt of molecule AA1 (3.30 g, 7.6 mmol) and to a poly-L-glutamic acid of relative number-average molecular weight (Mn)/mol 5200 g/mol (10.0 g) obtained by a process similar to that used in the preparation of co-polyamino acid AB1-1.

Dry extract: 23.4 mg/g
DP (estimated based on RMN ¹H): 35
The calculated average molar mass of the co-polyamino acid AB3 is 6594 g/mol.
Based on ¹H NMR: i=0.10
Aqueous HPLC-SEC (PEG calibrant): Mn=4900 g/mol.

### EXAMPLE AB4: CO-POLYAMINO ACID AB4—SODIUM POLY-L-GLUTAMATE MODIFIED BY MOLECULE AA2 AND HAVING A NUMBER-AVERAGE MOLECULAR WEIGHT (MN) OF 1800 G/MOL

By a process similar to that used in the preparation of co-polyamino acid AB1 applied to the hydrochloride salt of molecule AA2 (1.09 g, 2.4 mmol) and a poly-L-glutamic acid of average mass Mn=5600 g/mol (6.3 g) obtained by a process similar to that used in the preparation of co-polyamino acid AB1-1 but with a benzyl ester deprotection step using trimethylsilane iodide according to the protocol described in publication J. Am. Chem. Soc. 2000, 122, 26-34 (Subramanian G., et al.), A sodium poly-L-glutamate modified with AA2 molecule is obtained.

Dry extract: 21.5 mg/g
DP (estimated based on RMN ¹H): 35
Based on ¹H NMR: i=0.052
The calculated average molar mass of the co-polyamino acid AB4 is 6022 g/mol.
Aqueous HPLC-SEC (PEG calibrant): Mn=1800 g/mol.

### EXAMPLE AB5: CO-POLYAMINO ACID AB5—SODIUM POLY-L-GLUTAMATE MODIFIED BY MOLECULE AA6 AND HAVING A NUMBER-AVERAGE MOLECULAR WEIGHT (MN) OF 2600 G/MOL

A sodium poly-L-glutamate modified with molecule AA6 is obtained by a process similar to that used in the preparation of co-polyamino acid AB1 applied to the hydrochloride salt of molecule AA6 (2.06 g, 3.8 mmol) and to a poly-L-glutamic acid (9.8 g) obtained by a process similar to that used in the preparation of polyamino acid AB1-1.

Dry extract: 20.9 mg/g
DP (estimated based on RMN ¹H): 23
Based on ¹H NMR: i=0.05
The calculated average molar mass of the co-polyamino acid AB5 is 4079 g/mol.
Aqueous HPLC-SEC (PEG calibrant): Mn=2600 g/mol.

### EXAMPLE AB6: CO-POLYAMINO ACID AB6—SODIUM POLY-L-GLUTAMATE MODIFIED BY MOLECULE AA7 AND HAVING A NUMBER-AVERAGE MOLECULAR WEIGHT (MN) OF 4000 G/MOL

A poly-L-glutamic acid of average mass Mn=3500 g/mol and a polymerization degree of 22 (10.0 g) obtained by a process similar to that used in the preparation of co-polyamino acid AB1-1 is solubilized in DMF (420 mL) at 30-40° C. and maintained at this temperature. In parallel, the hydrochloride salt of the molecule AA7 (1.47 g, 2.3 mmol) is suspended in DMF (12 mL) and triethylamine (0.23 g, 2.3 mmol) is added then the mixture is gently heated with stirring until completely dissolved. NMM (7.6 g, 75 mmol), solution of AA7, then 2-hydroxypyridine N-oxide (HOPO, 0.84 g, 7.5 mmol) are successively added to the co-polyamino acid solution in DMF. The reaction medium is then cooled to 0° C., then EDC (1.44 g, 7.5 mmol) is added and the medium is raised to room temperature for 2 hours. The reaction medium is filtered through a 0.2 mm woven filter and poured drop by drop onto 3.5 L of water containing NaCl 15% by weight and HCl (pH 2) with stirring. At the end of the addition, the pH is readjusted to 2 with a 37% HCl solution, and the suspension is allowed to stand overnight. The precipitate is recovered by filtration, then rinsed with 100 ml of water. The white solid obtained is solubilized in 500 mL of water by slowly adding a 1N aqueous NaOH solution to pH 7 with stirring, then the solution is filtered through a 0.45 μm filter. The clear solution obtained is purified by ultrafiltration against 0.9% NaCl solution, then with water, until the conductimetry of the permeate is less than 50 μS/cm. The solution is filtered through a 0.2 μm filter and stored at 2-8° C.

Dry extract: 21.6 mg/g
DP (estimated based on RMN ¹H): 20
Based on ¹H NMR: i=0.025
The calculated average molar mass of the co-polyamino acid AB6 is 3369 g/mol.
Aqueous HPLC-SEC (PEG calibrant): Mn=4000 g/mol.

### EXAMPLE AB7: CO-POLYAMINO ACID AB7—SODIUM POLY-L-GLUTAMATE CAPPED AT ONE OF ITS ENDS BY AN ACETYL GROUP AND MODIFIED BY MOLECULE AA7 AND HAVING A NUMBER-AVERAGE MOLECULAR WEIGHT (MN) OF 3300 G/MOL

Co-Polyamino Acid AB7-1: Poly-L-Glutamic Acid of Number-Average Molar Mass (Mn) Relative to 3600 g/Mol and of DP 21 Resulting from the Polymerization of γ-Benzyl-L-Glutamate N-Carboxyanhydride Initiated by the Hexylamine and Capped at on One End by an Acetyl Group

In a previously oven-dried flask, γ-benzyl-L-glutamate N-carboxyanhydride (Glu (OBn)-NCA, 100.0 g, 380 mmol) is placed under vacuum for 30 minutes, then anhydrous DMF (225 mL) is added. The mixture is then stirred under argon until complete dissolution, cooled to 4° C., then hexylamine (1.78 g, 17 mmol) is quickly introduced. The mixture is stirred between 4° C. and room temperature for 2 days, then precipitated in diisopropyl ether (3.4 L). The precipitate is collected by filtration, washed twice with diisopropyl ether (225 mL), then dried to give a white solid which is dissolved in 450 mL of THF. DIPEA (31 mL, 176 mmol), then acetic anhydride (17 mL, 176 mmol) are successively added to this solution. After stirring overnight at room temperature, the solution is slowly poured into diisopropyl ether (3 L) under stirring. After stirring for 1 hour, the precipitate is filtered off, washed twice with diisopropyl ether (250 mL), then dried under vacuum at 30° C. to give a poly (gamma-benzyl-L-glutamic acid) capped on one of its ends. by an acetyl group.

A solution of the above hydrobromic acid (HBr) at 33% in acetic acid (235 mL) is added dropwise-to-a solution of the above co-polyamino acid (72 g) in trifluoroacetic acid (TFA, 335 mL) at 4° C. The mixture is stirred at room temperature for 3 hours 30 minutes and then poured dropwise onto a 1:1 (v/v) mixture of diisopropyl ether and water with stirring (4 L). After stirring for 2 hours, the heterogeneous mixture is allowed to stand overnight. The white precipitate is recovered by filtration, washed with a 1:1 (v/v) mixture of diisopropyl ether and water (340 mL), then with water (340 mL).

The obtained solid is then solubilized in water (1.5 L) by adjusting the pH to 7 by adding a 10N aqueous solution of sodium hydroxide, then a 1N aqueous sodium hydroxide solution. After solubilization, the solution is diluted by adding water to obtain a final volume of 2.1 L. The solution is filtered through a 0.45 μm filter, then purified by ultrafiltration against a solution of NaCl 0.9%, then water until the conductimetry of the permeate is less than 50 μS/cm. The solution of co-polyamino acid is then concentrated until a final volume of 1.8 L is obtained.

The aqueous solution is then acidified by adding a 37% hydrochloric acid solution until a pH of 2 is reached. After 4 hours of stirring, the precipitate obtained is filtered, washed with water (330 mL) and then dried under vacuum at 30° C. to produce a poly-L-glutamic acid of number-average molar mass (Mn) 3600 g/mol relative to a standard of polyoxyethylene (PEG), and average polymerization degree of 21.

### Co-Polyamino Acid AB7:

A sodium poly-L-glutamate acid modified with the molecule AA7 is obtained by a process similar to that used in the preparation of the co-polyamino acid AB6 applied to the hydrochloride salt of molecule AA7 (1.43 g, 2.2 mmol) and the co-polyamino acid AB7-1 (10.0 g).

Dry extract: 24.3 mg/g
DP (estimated based on RMN ¹H): 21
Based on ¹H NMR: i=0.03
The calculated average molar mass of the co-polyamino acid AB7 is 3677 g/mol.
Aqueous HPLC-SEC (PEG calibrant): Mn=3300 g/mol.

### EXAMPLE AB8: CO-POLYAMINO ACID AB8—SODIUM POLY-L-GLUTAMATE MODIFIED BY MOLECULE AA7 AND HAVING A NUMBER-AVERAGE MOLECULAR WEIGHT (MN) OF 3600 G/MOL

Co-Polyamino Acid AB8-1: Poly-L-Glutamic Acid of Number-Average Molar Mass (Mn) 3800 g/Mol and Degree of Polymerization 24 Resulting from the Polymerization of γ-Methyl-L-Glutamate N-Carboxyanhydride Initiated by Ammonia

A poly-L-glutamic acid is obtained by a process similar to that described in patent application FR-A-2 801 226 applied to γ-methyl-L-glutamic acid N-carboxyanhydride (25.0 g, 133.6 mmol) and 0.5 N ammonia solution in dioxane (12.1 mL, 6.05 mmol).

### Co-Polyamino Acid AB8:

A sodium poly-L-glutamate acid modified with the molecule AA7 is obtained by a process similar to that used in the preparation of the co-polyamino acid AB6 applied to the hydrochloride salt of molecule AA7 (2.1 g, 3.24 mmol) and co-polyamino acid AB8-1 (14.3 g).

Dry extract: 25.2 mg/g
DP (estimated based on RMN ¹H): 24
Based on ¹H NMR: i=0.03
The calculated average molar mass of the co-polyamino acid AB8 is 4099 g/mol.
Aqueous HPLC-SEC (PEG calibrant): Mn=3600 g/mol.

### EXAMPLE AB9: CO-POLYAMINO ACID AB9—SODIUM POLY-L-GLUTAMATE MODIFIED BY MOLECULE AA3 AND HAVING A NUMBER-AVERAGE MOLECULAR WEIGHT (MN) OF 3200 G/MOL

A sodium poly-L-glutamate modified by molecule AA3 is obtained by a process similar to the one used in the preparation of co-polyamino acid AB1 applied to the hydrochloride salt of molecule AA3 and to a poly-L-glutamic acid obtained by a process similar to the one used in the preparation of co-polyamino acid AB1-1.

Dry extract: 14.7 mg/g
DP (estimated based on RMN ¹H): 30
Based on ¹H NMR: i=0.12
The calculated average molar mass of the co-polyamino acid AB9 is 6192 g/mol.
Aqueous HPLC-SEC (PEG calibrant): Mn=3200 g/mol.

### EXAMPLE AB10: CO-POLYAMINO ACID AB10—SODIUM POLY-L-GLUTAMATE MODIFIED BY MOLECULE AA4 AND HAVING A NUMBER-AVERAGE MOLECULAR WEIGHT (MN) OF 2600 G/MOL

A sodium poly-L-glutamate modified by molecule AA4 is obtained by a process similar to the one used in the preparation of co-polyamino acid AB7 applied to the hydrochloride salt of molecule AA4 and to a poly-L-glutamic acid obtained by a process similar to the one used in the preparation of co-polyamino acid AB1-1.

Dry extract: 18.3 mg/g
DP (estimated based on RMN ¹H): 25
Based on ¹H NMR: i=0.08
The calculated average molar mass of the co-polyamino acid AB10 is 4870 g/mol.
Aqueous HPLC-SEC (PEG calibrant): Mn=2600 g/mol.

### EXAMPLE AB11: CO-POLYAMINO ACID AB11—SODIUM POLY-L-GLUTAMATE MODIFIED BY MOLECULE AA5 AND HAVING A NUMBER-AVERAGE MOLECULAR WEIGHT (MN) OF 2700 G/MOL

A sodium poly-L-glutamate modified by molecule AA5 is obtained by a process similar to the one used in the preparation of co-polyamino acid AB6 applied to the hydrochloride salt of molecule AA5 and to a poly-L-glutamic acid obtained by a process similar to the one used in the preparation of co-polyamino acid AB1-1.

Dry extract: 20.2 mg/g
DP (estimated based on RMN ¹H): 23
Based on ¹H NMR: i=0.05
The calculated average molar mass of the co-polyamino acid AB11 is 4072 g/mol.
Aqueous HPLC-SEC (PEG calibrant): Mn=2700 g/mol.

### EXAMPLE AB12: CO-POLYAMINO ACID AB12—SODIUM POLY-L-GLUTAMATE MODIFIED BY MOLECULE AA8 AND HAVING A NUMBER-AVERAGE MOLECULAR WEIGHT (MN) OF 3000 G/MOL

A sodium poly-L-glutamate modified by molecule AA8 is obtained by a process similar to the one used in the preparation of co-polyamino acid AB1 applied to the hydrochloride salt of molecule AA8 and to a poly-L-glutamic acid obtained by a process similar to the one used in the preparation of co-polyamino acid AB1-1.

Dry extract: 19.5 mg/g
DP (estimated based on RMN ¹H): 26
Based on ¹H NMR: i=0.04
The calculated average molar mass of the co-polyamino acid AB12 is 4477 g/mol.
Aqueous HPLC-SEC (PEG calibrant): Mn=3000 g/mol.

### EXAMPLE AB13: CO-POLYAMINO ACID AB13—SODIUM POLY-L-GLUTAMATE MODIFIED BY MOLECULE AA9 AND HAVING A NUMBER-AVERAGE MOLECULAR WEIGHT (MN) OF 3300 G/MOL

By a process similar to that used in the preparation of co-polyamino acid AB6 applied to the hydrochloride salt of molecule AA9 and a poly-L-glutamic acid obtained by a process similar to that used in the preparation of co-polyamino acid AB1-1 Using isoamylamine as the initiator in place of hexylamine, a sodium poly-L-glutamate modified with molecule AA9 is obtained.

Dry extract: 22.3 mg/g
DP (estimated based on RMN ¹H): 35
Based on ¹H NMR: i=0.12
The calculated average molar mass of the co-polyamino acid AB13 is 7226 g/mol.
Aqueous HPLC-SEC (PEG calibrant): Mn=3300 g/mol.

### EXAMPLE AB21: CO-POLYAMINO ACID AB21—SODIUM POLY-L-GLUTAMATE MODIFIED BY MOLECULE AA7 AND HAVING A NUMBER-AVERAGE MOLECULAR WEIGHT (MN) OF 3400 G/MOL

A sodium poly-L-glutamate modified with molecule AA7 is obtained by a process similar to that used in the preparation of co-polyamino acid AB6 applied to the hydrochloride salt of molecule AA7 (2.44 g, 2.4 mmol) and to a poly-L-glutamic acid (10 g) obtained by a process similar to that used in the preparation of polyamino acid AB1-1.

Dry extract: 22.7 mg/g
DP (estimated based on RMN ¹H): 22
Based on ¹H NMR: i=0.056
The calculated average molar mass of the co-polyamino acid AB21 is 4090 g/mol.
Aqueous HPLC-SEC (PEG calibrant): Mn=3400 g/mol.

### EXAMPLE AB22: CO-POLYAMINO ACID AB22—SODIUM POLY-L-GLUTAMATE CAPPED AT ONE OF ITS ENDS BY AN ACETYL GROUP AND MODIFIED BY MOLECULE AA10 AND HAVING A NUMBER-AVERAGE MOLECULAR WEIGHT (MN) OF 4000 G/MOL

The hydrochloride salt of molecule AA10 (4.56 g, 11.29 mmol) is dissolved in chloroform (60 mL) and triethylamine (1.14 g, 11.29 mmol) is added. NMM (7.6 g, 75.26 mmol), then HOPO (2.51 g, 22.58 mmol) are successively added to a co-polyamino acid (10.0 g, 75.3 mmol) solution obtained according to a process similar to that used in the preparation of co-polyamino acid B7-1 in DMF (420 mL). The reaction medium is then cooled to 0° C., then EDC (4.33 g, 22.58 mmol) is added, the medium is stirred for 1 h at 0° C., then the solution of the molecule AA10 is added. The reaction mixture is stirred for 2 hours at between 0° C. and room temperature. The reaction medium is filtered through a 0.2 mm woven filter and poured drop by drop onto 3.95 L of water containing NaCl 15% by weight and HCl (pH 2) with stirring. At the end of the addition, the pH is readjusted to 2 with a 37% HCl solution, and the suspension is allowed to stand overnight. The precipitate is recovered by filtration, then solubilized in 780 mL of water by slow addition of a 1N aqueous NaOH solution to pH 7 with stirring. After filtration through a 0.45 μm filter, the solution is diluted by adding water, then acetone is added to obtain a solution containing 30% mass of acetone. This solution is filtered through an activated charcoal filter, then the acetone is distilled (40° C., 100 mbar). After filtration through a 0.45 μm filter, the product is purified by ultrafiltration against a 0.9% NaCl aqueous solution, a carbonate buffer solution (150 mM), a 0.9% NaCl aqueous solution, a phosphate buffer (150 mM) solution, a 0.9% NaCl aqueous solution, then water until the conductimetry of the permeate is less than 50 μS/cm. The solution is then concentrated, filtered through a 0.2 m filter and stored at 2-8° C.

Dry extract: 19.7 mg/g
DP (estimated based on RMN ¹H): 38
Based on ¹H NMR: i=0.16
The calculated average molar mass of the co-polyamino acid AB22 is 7877 g/mol.
Organic HPLC-SEC (PEG Calibrator): Mn=4000 g/mol.

### EXAMPLE AB23: CO-POLYAMINO ACID AB23—SODIUM POLY-L-GLUTAMATE MODIFIED BY MOLECULE AA10 AND HAVING A NUMBER-AVERAGE MOLECULAR WEIGHT (MN) OF 7600 G/MOL

Co-Polyamino Acid AB23-1: Poly-L-Glutamic Acid from the Polymerization of γ-Benzyl-L-Glutamate N-Carboxyanhydride Initiated by Hexylamine and Capped at One End by a Pyroglutamate Group

A poly-L-glutamic acid (20.0 g) obtained by a process similar to that used in the preparation of the co-polyamino acid AB1-1 is solubilized in DMF at 80° C., then maintained at this temperature. After 24 hours, the reaction medium is poured into a solution of NaCl at 15% and at pH 2. After 4 hours, the white solid is recovered by filtration, rinsed with water, then dried under vacuum at 30° C.

### Co-Polyaminoamide AB23

A sodium poly-L-glutamate acid modified with the AA10 molecule is obtained by a process similar to that used in the preparation of the co-polyamino acid AB22 applied to the hydrochloride salt of molecule AA10 (2.742 g, 6.79 mmol) and the co-polyamino acid AB23-1 (9.0 g).

Dry extract: 21.9 mg/g
DP (estimated based on RMN ¹H): 60
Based on ¹H NMR: i=0.1
The calculated average molar mass of the co-polyamino acid AB23 is 11034 g/mol.
Organic HPLC-SEC (PEG Calibrator): Mn=7600 g/mol.

### EXAMPLE AB24: CO-POLYAMINO ACID AB24—SODIUM POLY-L-GLUTAMATE MODIFIED BY MOLECULE AA10 AND HAVING A NUMBER-AVERAGE MOLECULAR WEIGHT (MN) OF 4300 G/MOL

A sodium poly-L-glutamate modified by molecule AA10 is obtained by a process similar to the one used in the preparation of co-polyamino acid AB23 applied to the hydrochloride salt of molecule AA10 and to a poly-L-glutamic acid obtained by a process similar to the one used in the preparation of co-polyamino acid AB23-1.

Dry extract: 22.9 mg/g
DP (estimated based on RMN ¹H): 39
Based on ¹H NMR: i=0.15
The calculated average molar mass of the co-polyamino acid AB24 is 7870 g/mol.
Organic HPLC-SEC (PEG Calibrator): Mn=4300 g/mol.

### EXAMPLE AB25: CO-POLYAMINO ACID AB25—SODIUM POLY-L-GLUTAMATE MODIFIED BY MOLECULE AA10 AND HAVING A NUMBER-AVERAGE MOLECULAR WEIGHT (MN) OF 4200 G/MOL

A sodium poly-L-glutamate modified by molecule AA10 is obtained by a process similar to the one used in the preparation of co-polyamino acid AB23 applied to the hydrochloride salt of molecule AA10 and to a poly-L-glutamic acid obtained by a process similar to the one used in the preparation of co-polyamino acid AB23-1.

Dry extract: 25.9 mg/g
DP (estimated based on RMN ¹H): 39
Based on ¹H NMR: i=0.2
The calculated average molar mass of the co-polyamino acid AB25 is 8509 g/mol.
Organic HPLC-SEC (PEG Calibrator): Mn=4200 g/mol.

### EXAMPLE AB26: CO-POLYAMINO ACID AB26—SODIUM POLY-L-GLUTAMATE MODIFIED BY MOLECULE AA10 AND HAVING A NUMBER-AVERAGE MOLECULAR WEIGHT (MN) OF 2700 G/MOL

A sodium poly-L-glutamate modified by molecule AA10 is obtained by a process similar to the one used in the preparation of co-polyamino acid AB23 applied to the hydrochloride salt of molecule AA10 and to a poly-L-glutamic acid obtained by a process similar to the one used in the preparation of co-polyamino acid AB23-1.

Dry extract: 23.9 mg/g
DP (estimated based on RMN ¹H): 22
Based on ¹H NMR: i=0.21
The calculated average molar mass of the co-polyamino acid AB26 is 4899 g/mol.
Organic HPLC-SEC (PEG Calibrator): Mn=2700 g/mol.

### EXAMPLE AB27: CO-POLYAMINO ACID AB27—SODIUM POLY-L-GLUTAMATE MODIFIED BY MOLECULE AA11 AND HAVING A NUMBER-AVERAGE MOLECULAR WEIGHT (MN) OF 4500 G/MOL

A sodium poly-L-glutamate modified by molecule AA11 is obtained by a process similar to the one used in the preparation of co-polyamino acid AB23 applied to the hydrochloride salt of molecule AA11 and to a poly-L-glutamic acid obtained by a process similar to the one used in the preparation of co-polyamino acid AB23-1.

Dry extract: 26.8 mg/g
DP (estimated based on RMN ¹H): 39
Based on ¹H NMR: i=0.15
The calculated average molar mass of the co-polyamino acid AB27 is 8808 g/mol.
Organic HPLC-SEC (PEG Calibrator): Mn=4500 g/mol.

### EXAMPLE AB28: CO-POLYAMINO ACID AB28—SODIUM POLY-L-GLUTAMATE MODIFIED BY MOLECULE AA12 AND HAVING A NUMBER-AVERAGE MOLECULAR WEIGHT (MN) OF 4000 G/MOL

A sodium poly-L-glutamate modified by molecule AA12 is obtained by a process similar to the one used in the preparation of co-polyamino acid AB23 applied to the hydrochloride salt of molecule AA12 and to a poly-L-glutamic acid obtained by a process similar to the one used in the preparation of co-polyamino acid AB23-1.

Dry extract: 22.9 mg/g
DP (estimated based on RMN ¹H): 39
Based on ¹H NMR: i=0.15
The calculated average molar mass of the co-polyamino acid AB28 is 7706 g/mol.
Organic HPLC-SEC (PEG Calibrator): Mn=4000 g/mol.

### EXAMPLE AB29: CO-POLYAMINO ACID AB29—SODIUM POLY-L-GLUTAMATE MODIFIED BY MOLECULE AA13 AND HAVING A NUMBER-AVERAGE MOLECULAR WEIGHT (MN) OF 4000 G/MOL

Co-Polyamino Acid AB29-1: Poly-L-Glutamic Acid from the Polymerization of γ-Benzyl-L-Glutamate N-Carboxyanhydride Initiated by Hexylamine

In a double-jacket reactor, γ-benzyl-L-glutamate N-carboxyanhydride (500 g, 1.90 mol) is solubilized in anhydrous DMF (1100 mL). The mixture is then stirred until complete dissolution, cooled to 0° C., then hexylamine (6.27 mL, 47.5 mmol) is introduced rapidly. The mixture is stirred at 0° C. for 5 hours, between 0° C. and 20° C. for 7 hours, then at 20° C. for 7 hours. The reaction medium is then heated at 65° C. for 2 hours, cooled to 55° C. and methanol (3300 mL) is introduced after 1 hour 30 minutes. The reaction mixture is then cooled to 0° C. and left under stirring for 18 hours. The white precipitate is collected by filtration, washed with diisopropyl ether (2×800 mL), then dried under vacuum at 30° C. to give a poly (gamma-benzyl-L-glutamic acid) (PBLG).

Pd/Al₂O₃ (36 g) is added to a PBLG (180 g) solution in N,N-dimethylacetamide (DMAc, 450 mL) under an argon atmosphere. The mixture is placed in a hydrogen atmosphere (10 bar) and stirred at 60° C. for 24 hours. After cooling at room temperature and filtration of the catalyst on P4 sinter and PTFE Omnipore hydrophilic membrane 0.2 μm, a water solution at pH 2 (2700 mL) is poured dropwise on the DMAc solution, on a 45 min period with stirring. After stirring for 18 hours, the white precipitate is recovered by filtration, washed with water, then dried under reduced pressure at 30° C.

### Co-Polyamino Acid AB29

A sodium poly-L-glutamate modified by molecule AA13 is obtained by a process similar to that used in the preparation of co-polyamino acid AB23 applied to the hydrochloride salt of molecule AA13 and co-polyamino acid AB29-1.

Dry extract: 16.1 mg/g
DP (estimated based on RMN ¹H): 40
Based on ¹H NMR: i=0.15
The calculated average molar mass of the co-polyamino acid AB29 is 7734 g/mol.
Organic HPLC-SEC (PEG Calibrator): Mn=4000 g/mol.

### EXAMPLE AB30: CO-POLYAMINO ACID AB30—SODIUM POLY-L-GLUTAMATE MODIFIED BY MOLECULE AA10 AND HAVING A NUMBER-AVERAGE MOLECULAR WEIGHT (MN) OF 4300 G/MOL

A sodium poly-L-glutamate modified with molecule AA10 is obtained by a process similar to that used in the preparation of co-polyamino acid AB29 applied to the hydrochloride salt of molecule AA10 and a poly-L-glutamic acid obtained by a process similar to that used in the preparation of co-polyamino acid AB29-1 using molecule AA10 as the initiator in place of hexylamine.

Dry extract: 29.2 mg/g
DP (estimated based on RMN ¹H): 40
Based on ¹H NMR: i=0.125
The calculated average molar mass of the co-polyamino acid AB30 is 7682 g/mol.
Organic HPLC-SEC (PEG Calibrator): Mn=4300 g/mol.

### EXAMPLE AB31: CO-POLYAMINO ACID AB30—SODIUM POLY-L-GLUTAMATE MODIFIED BY MOLECULE AA10 AND HAVING A NUMBER-AVERAGE MOLECULAR WEIGHT (MN) OF 6300 G/MOL

A sodium poly-L-glutamate modified with molecule AA10 is obtained by a process similar to that used in the preparation of co-polyamino acid AB29 applied to the hydrochloride salt of molecule AA10 and a poly-L-glutamic acid obtained by a process similar to that used in the preparation of co-polyamino acid AB29-1 using molecule AA10 as the initiator in place of hexylamine.

Dry extract: 23.1 mg/g
DP (estimated based on RMN ¹H): 40
Based on ¹H NMR: i=0.175
The calculated average molar mass of the co-polyamino acid AB31 is 8337 g/mol.
Organic HPLC-SEC (PEG Calibrator): Mn=6300 g/mol.

### EXAMPLE AB32: CO-POLYAMINO ACID AB32—SODIUM POLY-L-GLUTAMATE MODIFIED BY MOLECULE AA14 AND HAVING A NUMBER-AVERAGE MOLECULAR WEIGHT (MN) OF 4700 G/MOL

A sodium poly-L-glutamate modified by molecule AA14 is obtained by a process similar to that used in the preparation of co-polyamino acid AB29 applied to molecule AA14 and poly-L-glutamic acid AB29-1.

Dry extract: 13.5 mg/g
DP (estimated based on RMN ¹H): 40
Based on ¹H NMR: i=0.109
The calculated average molar mass of the co-polyamino acid AB32 is 8599 g/mol.
Organic HPLC-SEC (PEG Calibrator): Mn=4700 g/mol.

### EXAMPLE AB38: CO-POLYAMINO ACID AB38—SODIUM POLY-L-GLUTAMATE MODIFIED BY MOLECULE AA18 AND HAVING A NUMBER-AVERAGE MOLECULAR WEIGHT (MN) OF 4700 G/MOL

A sodium poly-L-glutamate modified by molecule AA18 is obtained by a process similar to that used in the preparation of co-polyamino acid AB29 applied to molecule AA18 and poly-L-glutamic acid AB29-1.

Dry extract: 25 mg/g
DP (estimated based on RMN ¹H): 40
Based on ¹H NMR: i=0.15
The calculated average molar mass of the co-polyamino acid AB38 is 8954 g/mol.
Organic HPLC-SEC (PEG Calibrator): Mn=4700 g/mol.

### Co-Polyamino Acids According to Formula VII or VIIb

### EXAMPLE AB14: CO-POLYAMINO ACID AB14—SODIUM POLY-L-GLUTAMATE MODIFIED AT ONE OF ITS ENDS BY THE MOLECULE AA1 AND HAVING A NUMBER-AVERAGE MOLECULAR WEIGHT (MN) OF 3400 G/MOL

Hydrochloride salt of molecule AA1 (2.03 g, 4.70 mmol), chloroform (5 mL), molecular sieve 4 Å (1.3 g), as well as Amberlite IRN 150 ion exchange resin (1.3 g) are successively added to a suitable container. After 1 hour of stirring on rollers, the medium is filtered and the resin is rinsed with chloroform. The mixture is evaporated, then co-evaporated with toluene. The residue is solubilized in anhydrous DMF (30 mL) for direct use in the polymerization reaction.

γ-benzyl-L-glutamate N-carboxyanhydride Carboxyanhydride (25.59 g, 97.2 mmol) is placed under vacuum for 30 minutes in an oven-dried flask, then anhydrous DMF (140 mL) is added. The mixture is stirred under argon until complete solubilization, cooled to 4° C., then the solution of the molecule AA1 prepared as described above is rapidly introduced. The mixture is stirred at 4° C. and room temperature for 2 days, then heated at 65° C. for 2 hours. The reaction mixture is then cooled to room temperature, then poured dropwise into diisopropyl ether (1.7 L) with stirring. The white precipitate is recovered by filtration, washed twice with diisopropyl ether (140 mL), then dried under vacuum at 30° C. to obtain a white solid. The solid is diluted in TFA (160 mL), and a solution of 33% hydrobromic acid (HBr) in acetic acid (62 mL, 354 mmol) is then added dropwise—at 0° C. The solution is stirred for 2 hours at room temperature and is then poured dropwise on a mixture of 1:1 (v/v) diisopropyl ether/water and with stirring (1.9 L). After stirring for 2 hours, the heterogeneous mixture is allowed to stand overnight. The white precipitate is recovered by filtration, washed successively with a mixture 1: 1 (v/v) diisopropyl ether and water (280 mL) followed by water (140 mL). The obtained solid is solubilized in water (530 mL) by adjusting the pH to 7 by adding 10 N aqueous sodium hydroxide solution, then 1N aqueous sodium hydroxide solution. After solubilization, the theoretical concentration is adjusted to 20 g/L theoretical by addition of water to obtain a final volume of 800 mL. The mixture is filtered through a 0.45 μm filter, then purified by ultrafiltration against a 0.9% NaCl solution, then water until the conductimetry of the permeate is less than 50 μS/cm. The co-polyamino acid solution is then concentrated to about 30 g/L theoretical and the pH is adjusted to 7.0. The aqueous solution is filtered through 0.2 μm and stored at 4° C.

Dry extract: 24.1 mg/g
DP (estimated by ¹H) NMR=25 where i=0.04
The calculated average molar mass of the co-polyamino acid AB14 is 3378 g/mol.
Aqueous HPLC-SEC (PEG calibrant): Mn=3400 g/mol.

### EXAMPLE AB15: CO-POLYAMINO ACID AB15—SODIUM POLY-L-GLUTAMATE MODIFIED AT ONE OF ITS ENDS BY THE MOLECULE AA6 AND HAVING A NUMBER-AVERAGE MOLECULAR WEIGHT (MN) 4100 G/MOL

A poly-L-glutamate sodium modified on end by molecule AA6 is obtained by a process similar to that used in the preparation of co-polyamino acid AB14 applied to the hydrochloride salt of molecule AA6 (2.16 g, 3.94 mmol) and 25.58 g (97.2 mmol) of γ-benzyl-L-glutamate N-carboxyanhydride.

Dry extract: 45.5 mg/g
DP (estimated by ¹H) NMR=30 where i=0.033
The calculated average molar mass of the co-polyamino acid AB15 is 5005 g/mol.
Aqueous HPLC-SEC (PEG calibrant): Mn=4100 g/mol.

### EXAMPLE AB16: CO-POLYAMINO ACID AB16—SODIUM POLY-L-GLUTAMATE MODIFIED AT ONE END BY THE MOLECULE AA6 AND HAVING A NUMBER-AVERAGE MOLECULAR WEIGHT (MN) OF 6500 G/MOL

A poly-L-glutamate sodium modified on end by molecule AA6 is obtained by a process similar to that used in the preparation of co-polyamino acid AB14 applied to the hydrochloride salt of molecule AA6 (2.39 g, 4.36 mmol) and 50.0 g (189.9 mmol) of γ-benzyl-L-glutamate N-carboxyanhydride.

Dry extract: 28.5 mg/g
DP (estimated by ¹H) NMR=48 where i=0.021
The calculated average molar mass of the co-polyamino acid AB16 is 7725 g/mol.
Aqueous HPLC-SEC (PEG calibrant): Mn=6500 g/mol.

### EXAMPLE AB17: CO-POLYAMINO ACID AB17—SODIUM POLY-L-GLUTAMATE MODIFIED AT ONE END BY THE MOLECULE AA7 AND HAVING A NUMBER-AVERAGE MOLECULAR WEIGHT (MN) OF 3500 G/MOL

A poly-L-glutamate sodium modified at one of its end by molecule AA7 is obtained by a process similar to that used in the preparation of co-polyamino acid AB14 applied to the hydrochloride salt of molecule AA7 (2.80 g, 4.32 mmol) and 25.0 g (94.9 mmol) of γ-benzyl-L-glutamate N-carboxyanhydride.

Dry extract: 25.2 mg/g
DP (estimated by ¹H) NMR=26 where i=0.038
The calculated average molar mass of the co-polyamino acid AB17 is 4500 g/mol.
Aqueous HPLC-SEC (PEG calibrant): Mn=3500 g/mol.

### EXAMPLE AB18: CO-POLYAMINO ACID AB18—SODIUM POLY-L-GLUTAMATE MODIFIED AT ONE END BY THE MOLECULE AA7 AND HAVING A NUMBER-AVERAGE MOLECULAR WEIGHT (MN) OF 3700 G/MOL

A sodium poly-L-glutamate modified on one end by molecule AA7 is obtained by polymerization of glutamic acid γ-methyl N-carboxyanhydride (25.0 g, 133.6 mmol) using the hydrochloride salt of molecule AA7 (2.80 g, 4.32 mmol) as initiator and by deprotecting the methyl esters by using a 37% hydrochloric acid solution according to the process described in patent application FR-A-2 801 226.

Dry extract: 44.3 mg/g
DP (estimated by ¹H) NMR=22 where i=0.045
The calculated average molar mass of the co-polyamino acid AB18 is 3896 g/mol.
Aqueous HPLC-SEC (PEG calibrant): Mn=3700 g/mol.

### EXAMPLE AB19: CO-POLYAMINO ACID AB19—SODIUM POLY-L-GLUTAMATE MODIFIED AT ONE END BY THE MOLECULE AA6 AND HAVING A NUMBER-AVERAGE MOLECULAR WEIGHT (MN) OF 10500 G/MOL

A sodium poly-L-glutamate modified at one end by molecule AA6 is obtained by a process similar to that used in the preparation of co-polyamino acid AB14 applied to the hydrochloride salt of molecule AA6 (1.64 g, 2.99 mmol) and to au γ-benzyl-L-glutamate N-carboxyanhydride (49.3 g, 187 mmol).

Dry extract: 23.4 mg/g
DP (estimated by ¹H) NMR=65 where i=0.015
The calculated average molar mass of the co-polyamino acid AB19 is 10293 g/mol.
Aqueous HPLC-SEC (PEG calibrant): Mn=10500 g/mol.

### EXAMPLE AB20: CO-POLYAMINO ACID AB20—SODIUM POLY-L-GLUTAMATE CAPPED ON ONE END BY AN ACETYL GROUP AND MODIFIED AT ONE END BY MOLECULE AA6 AND HAVING A NUMBER-AVERAGE MOLECULAR WEIGHT (MN) OF 10,400 G/MOL

Hydrochloride salt of molecule AA6 (0.545 g, 1.00 mmol), chloroform (10 mL), molecular sieve 4 Å (3 g), as well as Amberlite IRN 150 ion exchange resin (3 g) are successively added to a suitable container. After 1 hour of stirring on rollers, the medium is filtered and the resin is rinsed with chloroform. The mixture is evaporated, then co-evaporated with toluene. The residue is solubilized in anhydrous DMF (10 mL) for direct use in the polymerization reaction.

γ-benzyl-L-glutamate N-carboxyanhydride (17.0 g, 64.6 mmol) is placed under vacuum for 30 minutes in an oven-dried flask, then anhydrous DMF (30 mL) is added. The mixture is stirred under argon until complete solubilization, cooled to 4° C., then the solution of molecule AA6 prepared as described above is rapidly introduced. The mixture is stirred between 4° C. and room temperature for 2 days, then precipitated in diisopropyl ether (0.6 L). The precipitate is collected by filtration, washed twice with diisopropyl ether (40 mL), then dried to give a white solid which is dissolved in 80 mL of THF. DIPEA (1.7 mL, 9.8 mmol), then acetic anhydride (0.9 mL, 9.5 mmol) are successively added to this solution. After stirring overnight at room temperature, the solution is slowly poured into diisopropyl ether (480 mL) over a period of 30 minutes with stirring. After 1 hour of stirring, the precipitate is filtered, washed twice with diisopropyl ether (80 ml), then dried under vacuum at 30° C. to obtain a poly (gamma-benzyl-L-glutamic acid) capped at one end by an acetyl group and modified at the other end by molecule AA6 in the form of a white solid.

The solid is diluted in TFA (65 mL), and a solution of 33% hydrobromic acid (HBr) in acetic acid (45 mL, 257.0 mmol) is then added dropwise—at 4° C. The solution is stirred for 2 hours at room temperature and is then poured dropwise on a mixture of 1:1 (v/v) diisopropyl ether/water and with stirring (780 mL). After stirring for 2 hours, the heterogeneous mixture is allowed to stand overnight. The white precipitate is recovered by filtration, washed successively with a mixture of 1:1 (v/v) diisopropyl ether and water (70 mL) and then with water (70 mL). The obtained solid is solubilized in water (300 mL) by adjusting the pH to 7 by adding 10 N aqueous sodium hydroxide solution, then 1N aqueous sodium hydroxide solution. After solubilization, the theoretical concentration is adjusted to 20 g/L theoretical by addition of water to obtain a final volume of 440 mL. The mixture is filtered through a 0.45 μm filter, then purified by ultrafiltration against a 0.9% NaCl solution, then water until the conductimetry of the permeate is less than 50 μS/cm. The co-polyamino acid solution is then concentrated to about 30 g/L theoretical and the pH is adjusted to 7.0. The aqueous solution is filtered through 0.2 μm and stored at 4° C.

Dry extract: 21.5 mg/g
DP (estimated by ¹H) NMR=60 where i=0.017
The calculated average molar mass of the co-polyamino acid AB20 is 9619 g/mol.
Aqueous HPLC-SEC (PEG calibrant): Mn=10400 g/mol.

### EXAMPLE AB33: CO-POLYAMINO ACID AB33—SODIUM POLY-L-GLUTAMATE MODIFIED AT ONE END BY THE MOLECULE AA15 AND HAVING A NUMBER-AVERAGE MOLECULAR WEIGHT (MN) OF 1800 G/MOL

By a process similar to that used in the preparation of co-polyamino acid AB14 applied to molecule AA15 (0.82 g, 1.34 mmol) and 7.75 g (29.4 mmol) of γ-benzyl-L-glutamate N-carboxyanhydride, a solution of sodium poly-L-glutamate modified at one end by molecule AA15 is obtained.

Dry extract: 16.8 mg/g
DP (estimated by ¹H) NMR=22 where i=0.045
The calculated average molar mass of the co-polyamino acid AB33 is 3897 g/mol.
Aqueous HPLC-SEC (PEG calibrant): Mn=1800 g/mol.

### EXAMPLE AB34: CO-POLYAMINO ACID AB34—SODIUM POLY-L-GLUTAMATE MODIFIED AT ONE END BY THE MOLECULE AA4 AND HAVING A NUMBER-AVERAGE MOLECULAR WEIGHT (MN) OF 2600 G/MOL

In a previously oven-dried flask, γ-benzyl-L-glutamate N-carboxyanhydride (70.9 g, 269.3 mmol) is solubilized in anhydrous DMF (125 mL). The mixture is cooled to 4° C., then a solution of molecule AA4 in the form of neutral amine (6.80 g, 12.23 mmol) in DMF (35 mL) is introduced rapidly. The mixture is stirred between 4° C. and room temperature for 18 h, then heated at 65° C. for 2 hours. The reaction mixture is then cooled to room temperature, then poured dropwise into diisopropyl ether (2.4 L) with stirring. The white precipitate is recovered by filtration, washed with diisopropyl ether (2×125 mL), then dried under reduced pressure at 30° C. to give a white solid. The solid is solubilized in N,N-dimethylacetamide (DMAc, 150 mL), then Pd/Al₂O₃ (6 g) is added under an argon atmosphere. The mixture is placed in a hydrogen atmosphere (10 bar) and stirred at 60° C. for 24 hours. After cooling to room temperature and filtration of the catalyst on P4 sinter and PTFE Omnipore hydrophilic membrane 0.2 μm, a water solution at pH 2 (900 mL) is poured dropwise on the DMAc solution, on a 45 min period while stirring. After 18 hours of, the white precipitate is recovered by filtration, washed with water, then dried under reduced pressure at 30° C. The obtained solid is solubilized in water (1.25 L) by adjusting the pH to 7 by addition of a 1N aqueous sodium hydroxide solution. The pH is then adjusted to pH 12 and the solution is kept under stirring for 1 hour. After neutralization at pH 7, the solution is filtered through 0.2 μm, diluted with ethanol to obtain a solution containing 30% mass of ethanol, then filtered on an activated carbon filter (3M R53SLP). The solution obtained is filtered through 0.45 μm and purified by ultrafiltration against a 0.9% NaCl solution, then water until the conductimetry of the permeate is less than 50 μS/cm. The co-polyamino acid solution is then concentrated to about theoretical 30 g/L and the pH is adjusted to 7. The aqueous solution is filtered through 0.2 μm and stored at 4° C.

Dry extract: 38.1 mg/g
DP (estimated by ¹H) NMR=23 where i=0.043
The calculated average molar mass of the co-polyamino acid AB34 is 3991 g/mol.
Organic HPLC-SEC (PEG Calibrator): Mn=2600 g/mol.

### EXAMPLE AB35: CO-POLYAMINO ACID AB35—SODIUM POLY-L-GLUTAMATE MODIFIED AT ONE END BY THE MOLECULE AA14 AND HAVING A NUMBER-AVERAGE MOLECULAR WEIGHT (MN) OF 2600 G/MOL

A solution of sodium poly-L-glutamate modified at one end by molecule AA14 is obtained by a process similar to that used in the preparation of co-polyamino acid AB34 applied to molecule AA14 (0.4 g, 0.65 mmol) in solution in chloroform (6.5 mL) and 3.79 g (14.4 mmol) of γ-benzyl-L-glutamate N-carboxyanhydride dissolved in DMF (6.5 mL), and skipping the activated carbon filtration stop.

Dry extract: 21.0 mg/g
DP (estimated by ¹H) NMR=22 where i=0.045
The calculated average molar mass of the co-polyamino acid AB35 is 3896 g/mol.
Organic HPLC-SEC (PEG Calibrator): Mn=2600 g/mol.

### EXAMPLE AB36: CO-POLYAMINO ACID AB36—SODIUM POLY-L-GLUTAMATE MODIFIED AT ONE END BY THE MOLECULE AA16 AND HAVING A NUMBER-AVERAGE MOLECULAR WEIGHT (MN) OF 2800 G/MOL

By a process similar to that used in the preparation of co-polyamino acid AB34 applied to molecule AA16 (3.28 g, 4.67 mmol) and 27.02 g (102.6 mmol) of γ-benzyl-L-glutamate N-carboxyanhydride, a solution of sodium poly-L-glutamate modified at one end by molecule AA16 is obtained.

Dry extract: 23.9 mg/g
DP (estimated by ¹H) NMR=22 where i=0.045
The calculated average molar mass of the co-polyamino acid AB36 is 3987 g/mol.
Organic HPLC-SEC (PEG Calibrator): Mn=2800 g/mol.

### EXAMPLE AB37: CO-POLYAMINO ACID AB37—SODIUM POLY-L-GLUTAMATE MODIFIED AT ONE OF ITS ENDS BY THE MOLECULE AA17 AND HAVING A NUMBER-AVERAGE MOLECULAR WEIGHT (MN) OF 2800 G/MOL

By a process similar to that used in the preparation of co-polyamino acid AB34 applied to molecule AA17 (4.50 g, 9.73 mmol) and 56.33 g (214.0 mmol) of γ-benzyl-L-glutamate N-carboxyanhydride, a solution of sodium poly-L-glutamate modified at one end by molecule AA17 is obtained.

Dry extract: 26.8 mg/g
DP (estimated by 1H) NMR=24 where i=0.042
The calculated average molar mass of the co-polyamino acid AB37 is 4049 g/mol.
Organic HPLC-SEC (PEG Calibrator): Mn=2800 g/mol.

### Part B

### BB: Synthesis of Intermediate Hydrophobic Hy Compounds to Obtain the -Hy Radicals Wherein p=2

The hydrophobic intermediate compounds are represented in the following table by the corresponding hydrophobic molecule before co-polyamino acid grafting.

TABLE 1d
List of hydrophobic intermediates synthesized according to the invention.

| No | HYDROPHOBIC INTERMEDIATE COMPOUNDS |
|---|---|
| BA1 | 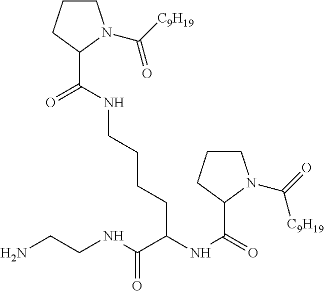 |
| BA2 | 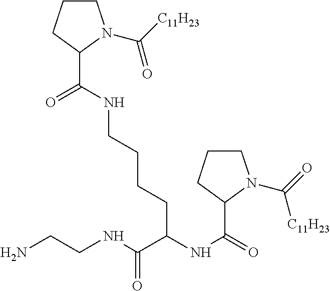 |
| BA3 | 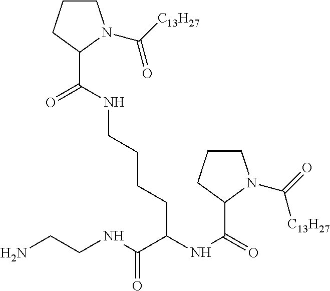 |
| BA4 | 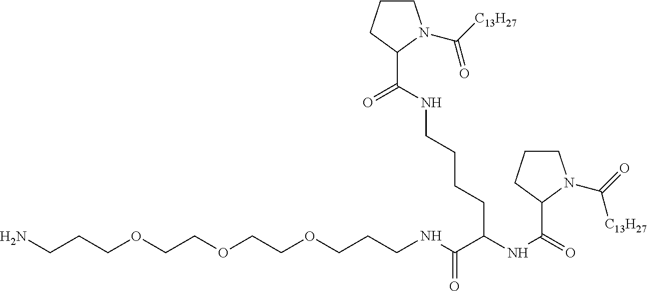 |
| BA5 | 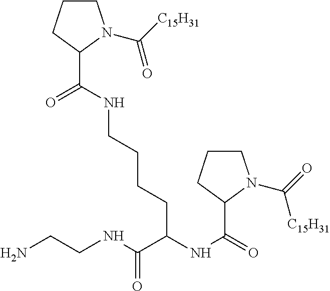 |
| BA6 | 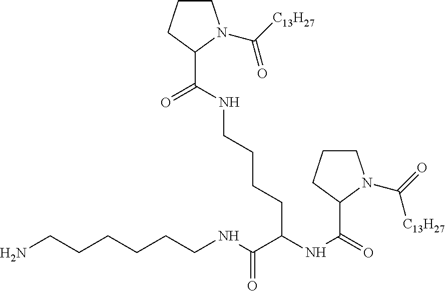 |
| BA7 | 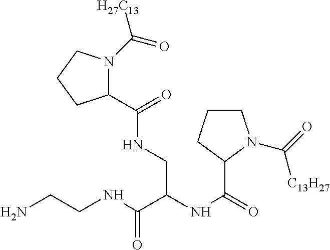 |

### Part BA: Synthesis of Hydrophobic Intermediates Wherein p=2

### EXAMPLE BA1: MOLECULE BA1

### Molecule B1: Product Obtained by the Reaction Between Decanoic Acid and L-Proline.

Dicyclohexyl carbodiimide (DCC) (16.29 g, 78.96 mmol) and N-hydroxysuccinimide (NHS) (9.09 g, 78.96 mmol) are successively added to a solution of decanoic acid (14.28 g, 82.91 mmol) in THF (520 mL) at 0° C. After stirring for 60 hours at room temperature, the medium is cooled to 0° C. for 20 minutes, filtered on sinter. L-proline (10 g, 86.86 mmol), diisopropylethylamine (DIPEA) (68.8 mL) and water (60 mL) are added to the filtrate. After stirring for 24 hours at room temperature, the medium is diluted with water (300 mL). The aqueous phase is washed with ethyl acetate (2×250 ml), acidified to pH ˜1 with a 1N HCl aqueous solution, then extracted with dichloromethane (3×150 ml). The combined organic phases are dried over Na₂SO₄, filtered, concentrated under vacuum, and the residue is purified by chromatography on silica gel (cyclohexane, ethyl acetate).

Yield: 14.6 g (69%)

NMR ¹H (CDCl₃, ppm): 0.87 (3H); 1.26 (12H); 1.65 (2H); 2.02 (3H); 2.34 (211); 2.41 (1H); 3.48 (1H); 3.56 (1H); 4.58 (1H).

LC/MS (ESI): 270.2; (calculated ([M+H]): 270.4).

### Molecule B2: Product Obtained by the Reaction Between Molecule B1 and L-Lysine.

By a process similar to the one used in the preparation of molecule B1 applied to molecule B1 (14.57 g, 54.07 mmol) and to L-lysine (4.15 g, 28.39 mmol), a yellow oil is obtained.

Yield: 16.4 g (93%)

NMR ¹H (CDCl₃, ppm): 0.88 (6H); 1.26 (24H); 1.35-1.65 (8H); 1.85-2.35 (12H); 2.53 (0.2H); 2.90 (0.8H); 3.45-3.75 (5H); 4.50-4.70 (3H); 7.82 (III).

LC/MS (ESI): 649.6; (calculated ([M+H]⁺): 649.9).

### Molecule B3: Product Obtained by Reaction Between Molecule B2 and N-Boc-Ethylenediamine.

DIPEA (8.80 mL) and 2-(1H-benzotriazol-1-yl)-1 1,3,3-tetramethyluronium tetrafluoroborate (TBTU, 8.52 g, 26.54 mmol) are added at room temperature to a solution of molecule B2 (16.4 g, 25.27 mmol) in THF (170 mL). After stirring for 30 minutes, BocEDA (4.45 g, 27.8 mmol) is added. After stirring at room temperature for 2 hours, the solvent is evaporated under reduced pressure and the residue is diluted with ethyl acetate (400 mL). The organic phase is washed with water (250 mL), saturated aqueous solution of NaHCO₃ (250 ml), an aqueous solution of 1 N HCl (250 ml), a saturated aqueous solution of NaCl (250 ml) and is dried over Na₂SO₄. After filtration and concentration under vacuum, the residue obtained is purified by chromatography on silica gel (ethyl acetate, methanol) to produce a colorless oil.

Yield: 12.8 g (64%)

NMR ¹H (CDCl₃, ppm): 0.87 (6H); 1.25-1.60 (42H); 1.80-2.05 (4H); 2.15-2.45 (9H); 3.10-3.75 (10H); 4.30 (1H); 4.50 (2H); 5.50 (0.6H); 5.89 (0.2H); 6.15 (0.2H); 7.03 (HI); 7.47 (1H).

LC/MS (ESI): 791.8; (calculated ([M+H]⁺): 792.1).

### Molecule BA1

A 4 N HCl solution in dioxane (20.2 mL) is added to a molecule B3 (12.78 g, 16.15 mmol) solution in dichloromethane (110 mL) at 5° C. After 20 hours of stirring at 5° C., the medium is concentrated under vacuum. The residue obtained is dissolved in methanol and evaporated under vacuum, this operation being repeated 4 times to give a white solid of molecule BA1 in the form of hydrochloride salt.

Yield: 11.4 g (97%)

NMR ¹H (DMSO-d₆, ppm): 0.85 (6H); 1.25-1.50 (33H); 1.57 (1H); 1.70-2.40 (12H); 2.82 (2H); 3.00 (2H); 3.25-3.70 (6H); 4.05-4.50 (3H); 7.75-8.45 (6H). LC/MS (ESI): 691.6; (calculated ([M+H]+): 692.0).

### EXAMPLE BA2: MOLECULE BA2

### Molecule B4: Product Obtained by the Reaction Between Lauric Acid and L-Proline.

By a process similar to the one used in the preparation of molecule B1, applied to lauric acid (31.83 g, 157.9 mmol) and to L-proline (20 g, 173.7 mmol), a yellow oil is obtained.

Yield: 34.3 g (73%)

NMR ¹H (CDCl₃, ppm): 0.87 (3H); 1.26 (16H); 1.70 (2H); 1.90-2.10 (3H); 2.35 (2H); 2.49 (1H); 3.48 (1H); 3.56 (1H); 4.60 (1H).

LC/MS (ESI): 298.2; (calculated ([M+1H]): 298.4).

### Molecule B5: Product Obtained by the Reaction Between Molecule B4 and L-Lysine.

A white solid is obtained by a process similar to the one used in the preparation of molecule B1 applied to molecule B4 (33.72 g, 113.36 mmol) and to L-lysine (8.70 g, 59.51 mmol).

Yield: 26.2 g (66%)

NMR ¹H (CDCl₃, ppm): 0.88 (6H); 1.26 (32H); 1.35-1.65 (8H); 1.85-2.35 (15H); 2.87 (1H); 3.40-3.75 (5H); 4.50-4.75 (3H); 7.87 (1H).

LC/MS (ESI): 705.6; (calculated ([M+H]⁺): 706.0).

### Molecule B6: Product Obtained by Reaction Between N-Boc-Ethylenediamine and Molecule B5.

A colorless oil colorless is obtained by a process similar to that used in the preparation of molecule B3 applied to molecule B5 (25.74 g, 36.51 mmol) and BocEDA (6.43 g, 40.16 mmol).

Yield: 30.9 g (quantitative)

NMR ¹H (CDCl₃, ppm): 0.88 (6H); 1.35-1.65 (50H); 1.85-2.35 (13H); 3.05-3.75 (10H); 4.25-4.65 (3H); 5.50 (0.4H); 5.88 (0.2H); 6.16 (0.2H); 7.08 (1H); 7.26 (1H); 7.49 (0.2H).

LC/MS (ESI): 847.8; (calculated ([M+H]⁺): 848.2).

### Molecule BA2

Following a process similar to the one used in the preparation of molecule BA1 applied to molecule B6 (30.9 g, 36.47 mmol), the residue obtained after concentration under vacuum is dissolved in methanol and evaporated under vacuum, this operation being repeated 4 times to yield a white solid of molecule BA2 in the form of a hydrochloride salt after drying under reduced pressure.

Yield: 27.65 g (97%)

NMR ¹H (DMSO-d₆, ppm): 0.85 (6H); 1.10-2.40 (54H); 2.75-3.15 (4H); 3.25-3.60 (6H); 4.05-4.50 (3H); 7.50-8.50 (6H).

LC/MS (ESI): 747.6; (calculated ([M+H]⁺): 748.1).

### EXAMPLE BA3: MOLECULE BA3

### Molecule B7: Product Obtained by the Reaction Between Myristic Acid and L-Proline.

A yellow oil is obtained by a process similar to the one used in the preparation of molecule B1, applied to lauric acid (18.93 g, 82.91 mmol) and to L-proline (10 g, 86.86 mmol).

Yield: 20 g (78%)

NMR ¹H (CDCl₃, ppm): 0.88 (3H); 1.28 (20H); 1.70 (2H); 1.90-2.10 (3H); 2.36 (2H); 2.51 (1H); 3.47 (1H); 3.56 (1H); 4.61 (H).

LC/MS (ESI): 326.2; (calculated ([M+H]⁺): 326.6).

### Molecule B8: Product Obtained by the Reaction Between Molecule B7 and L-Lysine

A white solid is obtained by a process similar to the one used in the preparation of molecule B1 applied to molecule B7 (20.02 g, 61.5 mmol) and to L-lysine (4.72 g, 32.29 mmol).

Yield: 12.3 g (53%)

NMR ¹H (DMSO-d₆, ppm): 0.85 (6H); 1.26 (40H); 1.35-1.50 (6H); 1.50-2.10 (10H); 2.10-2.25 (4H); 3.01 (2H); 3.31-3.55 (4H); 4.10-4.40 (3H); 7.68 (0.6H); 7.97 (11); 8.27 (0.4H); 12.50 (1H).

LC/MS (ESI): 761.8; (calculated ([M+H]+): 762.1).

### Molecule B9: Product Obtained by Reaction Between N-Boc-Ethylenediamine and Molecule B8.

By a process similar to the one used in the preparation of molecule B3 applied to molecule B8 (12 g, 15.77 mmol) and BocEDA (3.03 g, 18.92 mmol), a colorless oil is obtained after purification by chromatography column on silica gel (dichloromethane, methanol).

Yield: 12.5 g (88%)

NMR ¹H (DMSO-d₆, ppm): 0.85 (6H); 1.20-1.55 (55H); 1.50-2.25 (14H); 2.95-3.10 (6H); 3.31-3.55 (4H); 4.10-4.40 (3H); 6.74 (1H); 7.60-8.25 (3H).

LC/MS (ESI): 904.1; (calculated ([M+H]⁺): 904.3).

### Molecule BA3

Following a process similar to the one used in the preparation of molecule BA1 applied to molecule B9 (12.5 g, 13.84 mmol), the residue obtained after concentration under vacuum is dissolved in methanol and evaporated under vacuum, this operation being repeated 4 times to yield a white solid of molecule BA3 in the form of a hydrochloride salt after drying under reduced pressure.

Yield: 9.2 g (79%)

NMR ¹H (DMSO-d₆, ppm): 0.85 (6H); 1.10-1.65 (48H); 1.70-2.35 (12H); 2.85 (2H); 3.01 (2H); 3.25-3.65 (6H); 4.10-4.50 (3H); 7.70-8.40 (6H).

LC/MS (ESI): 803.9; (calculated ([M+H]⁺): 804.2).

### EXAMPLE BA4: MOLECULE BA4

### Molecule B10: Product Obtained by Reaction Between Molecule B8 and Boc-1-Amino-4,7,10-Trioxa-13-Tridecane Amine.

A thick colorless oil is obtained by a process similar to that used in the preparation of molecule B3 applied to molecule B8 (29.80 g, 39.15 mmol) and Boc-1-amino-4,7,10-trioxa-13-tridecane amine (15.05 g, 46.96 mmol).

Yield: 25.3 g (61%)

NMR ¹H (DMSO-d₆, ppm): 0.85 (6H); 1.25-2.35 (75H); 2.85-3.20 (6H); 3.25-3.65 (16H); 4.10-4.45 (3H); 6.38 (0.1H); 6.72 (0.9H); 7.50-8.25 (3H).

LC/MS (ESI): 1064.2; (calculated ([M+H]⁺): 1064.5).

### Molecule BA4

Following a process similar to the one used in the preparation of molecule BA1 applied to molecule B10 (25.3 g, 23.8 mmol), the residue obtained after concentration under vacuum is dissolved in methanol and evaporated under vacuum, this operation being repeated 4 times to yield a white solid of molecule BA4 in the form of a hydrochloride salt after drying under reduced pressure.

Yield: 20.02 g (84%)

NMR ¹H (DMSO-d₆, ppm): 0.85 (6H); 1.15-2.35 (66H); 2.80-3.20 (6H); 3.30-3.65 (16H); 4.10-4.45 (3H); 7.55-8.60 (6H).

LC/MS (ESI): 964.9; (calculated ([M+H]+): 964.6).

### EXAMPLE BA5: MOLECULE BA5

### Molecule B11: Product Obtained by Reaction Between Molecule A1 and L-Lysine

By a process similar to that used in the preparation of molecule BI applied to molecule A1 (19.10 g, 54.02 mmol) and L-lysine (4.15 g, 28.36 mmol), an oily residue is obtained after concentration of the reaction medium under reduced pressure. This residue is diluted in water (150 mL), washed with ethyl acetate (2×75 mL), then the aqueous phase is acidified to pH 1 by slow addition of 6N HCl. The product is extracted 3 times with dichloromethane, the organic phase is dried over Na₂SO₄ then filtered and concentrated under reduced pressure to give 11.2 g of the yellow oily residue. Simultaneously, the organic phase of the above ethyl acetate is washed with an aqueous solution of 2N HCl (2×75 ml), a saturated aqueous solution of NaCl (75 ml), dried over Na₂SO₄ filtered and concentrated to give 10.2 g of yellow oily residue. A white solid is obtained after recrystallization of each of these residues in acetone.

Yield: 11.83 g (54%)

¹H NMR (CDCl₃, ppm): 0.87 (6H); 1.06-2.44 (70H); 2.78-2.96 (1H); 3.35-3.75 (5H); 4.28-4.43 (0.1H); 4.43-4.52 (0.2H); 4.52-4.61 (1.8H); 4.61-4.75 (0.9H); 7.74-8.02 (2H).

LC/MS (ESI): 818.0; (calculated ([M+H]+): 818.7).

### Molecule B12: Product Obtained by Coupling Between Molecule B11 and N-Boc-Ethylenediamine

By a process similar to that used in the preparation of molecule B3 applied to molecule B11 (18.00 g, 22.02 mmol) solution in THF and BocEDA (4.23 g, 26.43 mmol), a white solid is obtained after 2 recrystallizations in acetonitrile.

Yield: 17.5 g (83%)

¹H NMR (DMSO-d6, ppm): 0.85 (611); 1.15-2.29 (79H); 2.92-3.12 (61); 3.30-3.59 (4H); 4.06-4.13 (0.65H); 4.16-4.29 (2H); 4.38-4.42 (0.35H); 6.71-6.76 (18); 7.60-7.69 (1.3H); 7.76-7.81 (0.65H); 7.93-7.97 (0.35H); 8.00-8.04 (0.35H); 8.10-8.17 (0.35H).

LC/MS (ESI): 960.4; (calculated ([M+H]⁺): 960.8).

### Molecule BA5

By a process similar to that used in the preparation of molecule BA1 applied to molecule B12 (24.4 g, 25.43 mmol), the residue obtained after concentration in vacuum is solubilized in dichloromethane (150 mL), the organic phase is washed twice with 2N aqueous sodium hydroxide solution (90 mL). Acetonitrile (120 mL) is added and dichloromethane is removed by concentration under reduced pressure. The medium is then left standing for 72 hours and a white solid is obtained after filtration and rinsing with acetonitrile, then drying under reduced pressure. This operation is repeated 4 times.

Yield: 14.28 g (65%)

¹H NMR (DMSO-d6, ppm): 0.85 (6H); 1.06-2.32 (70H); 2.53-2.63 (2H); 2.89-3.61 (10H); 4.04-4.43 (3H); 7.55-7.62 (0.65H); 7.65-7.72 (0.65H); 7.80 (0.65H); 7.91 (0.35H); 8.03 (0.35H); 8.14-8.23 (0.35H).

LC/MS (ESI): 860.0; (calculated ([M+H]⁺): 860.8).

### EXAMPLE BA6: MOLECULE BA6

### Molecule B13: Product Obtained by the Reaction Between N-(Tert-Butoxycarbonyl)-1,6-Diaminohexane and Molecule B8.

By a process similar to the one used in the preparation of molecule B3 applied to molecule B8 (10 g, 13.14 mmol) and to N-(tert-butoxycarbonyl)-1.6-diaminohexane (3.41 g, 15.77 mmol) in dichloromethane, a white solid is obtained after recrystallization in acetonitrile.

Yield: 10.7 g (85%)

NMR ¹H (CDCl₃, ppm): 0.88 (6H); 1.17-2.40 (79H); 3.00-3.71 (10H); 4.26-4.58 (3H); 4.67 (1H); 6.74 (1H); 7.34-7.49 (2H).

LC/MS (ESI): 959.9; (calculated ([M+H]⁺): 959.8).

### Molecule BA6

Following a process similar to that used in the preparation of molecule BA applied to molecule B13 (10.5 g, 10.94 mmol), an aqueous solution of 2N NaOH is added dropwise to the reaction medium cooled to 0° C. The aqueous phase is extracted with dichloromethane and the organic phase is washed thrice with 5% aqueous NaCl solution. After drying over Na₂SO₄, the organic phase is filtered, concentrated under vacuum, and the residue is recrystallized in acetonitrile.

Yield: 5.4 g (58%)

NMR ¹H (CDCl₃, ppm): 0.88 (6H); 1.19-2.40 (72H); 2.67 (2H); 3.03-3.70 (8H); 4.26-4.57 (3H); 6.71 (1H); 7.39-7.49 (2H).

LC/MS (ESI): 859.8; (calculated ([M+H]+): 859.7).

### EXAMPLE BA7: MOLECULE BA7

### Molecule B14: Product Obtained by Coupling Between Molecule B7 and 2,3-Diaminopropionic Acid

By a process similar to the one used in the preparation of molecule B1 applied to molecule B7 (80.00 g, 245.78 mmol) and to the dihydrochloride of 2,3-diaminopropionic acid (22.84 g, 129.04 mmol), a white solid is obtained after recrystallization in acetonitrile.

Yield: 69 g (78%)

¹H NMR (DMSO-d6, ppm): 0.86 (6H); 1.08-1.38 (40H); 1.40-1.55 (4H); 1.68-2.30 (12H); 3.16-3.66 (6H); 4.20-4.39 (3H); 7.67-8.31 (2H); 12.70 (1H).

LC/MS (ESI): 719.4; 741.5; (calculated ([M+H]⁺): 719.6; ([M+Na]⁺: 741.6).

### Molecule B15: Product Obtained by Coupling Between Molecule B114 and N-Boc-Ethylenediamine

By a process similar to that used in the preparation of molecule B3 applied to molecule B14 (32.00 g, 44.50 mmol) solution in dichloromethane and BocEDA (8.56 g, 53.40 mmol), a colorless oil is obtained after purification by chromatography on silica gel (ethyl acetate, methanol).

Yield: 24.5 g (64%)

¹H NMR (DMSO-d6, ppm): 0.85 (6H); 1.16-2.42 (65H); 2.89-3.14 (411); 3.17-3.66 (6H); 4.11-4.43 (3H); 6.77 (1H); 7.38-8.23 (3H).

LC/MS (ESI): 861.7; (calculated ([M+H]⁺): 861.7).

### Molecule BA7

After a process similar to that used in the preparation of molecule BA1 applied to molecule B15 (24.50 g, 28.45 mmol), the reaction medium is concentrated under reduced pressure, the residue is solubilized in dichloromethane, the phase organic is washed with an aqueous solution of NaOH 2 N), dried over Na₂SO₄, filtered and concentrated under reduced pressure. A white solid is obtained after recrystallization in acetonitrile.

Yield: 19.7 g (91%)

¹H NMR (DMSO-d6, ppm): 0.85 (6H); 1.10-2.40 (58H); 2.51-2.62 (2H); 2.90-3.16 (2H); 3.16-3.67 (6H); 4.04-4.47 (3H); 7.33-8.27 (3H).

LC/MS (ESI): 761.5; (calculated ([M+H]⁺): 761.6).

### BB: Synthesis of Co-Polyamino Acids

### Co-Polyamino Acids According to Formula VII or VIIa

TABLE 1e
List of co-polyamino acids synthesized according to the invention

| No | CO-POLYAMINO ACIDS BEARING CARBOXYLATE CHARGES AND HYDROPHOBIC RADICALS |
|---|---|
| BB1 | 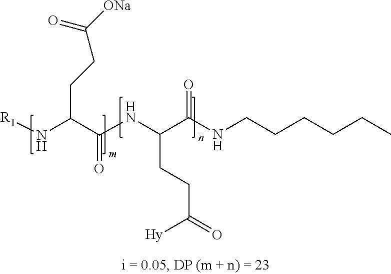 |
| | 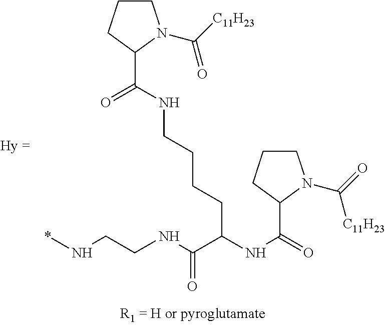 |
| BB2 | 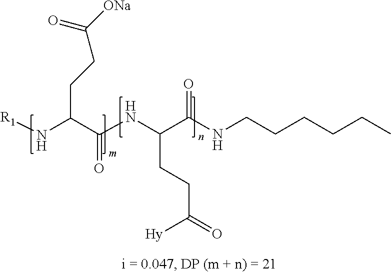 |
| | 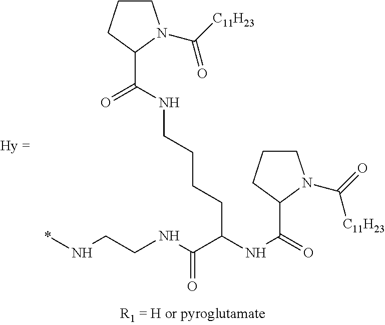 |
| BB3 | 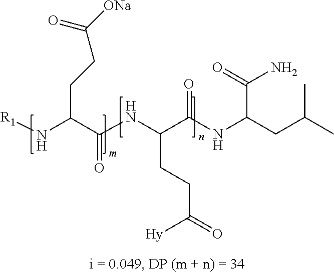 |
| | 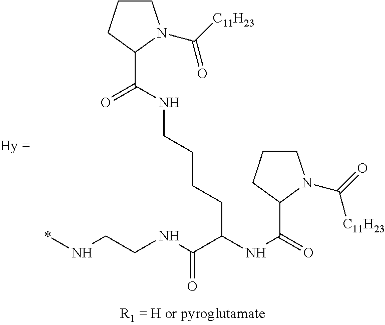 |
| BB4 | 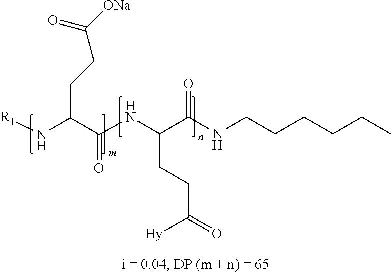 |
| | 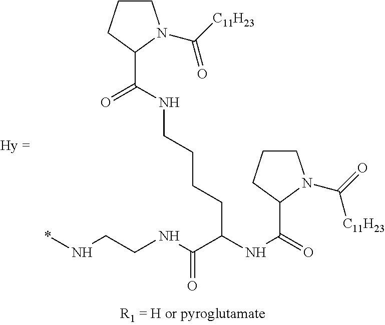 |
| BB5 | 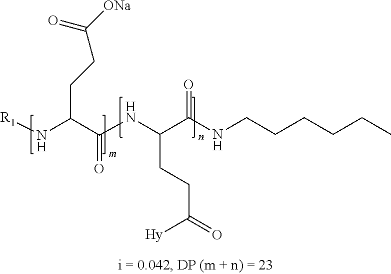 |
| | 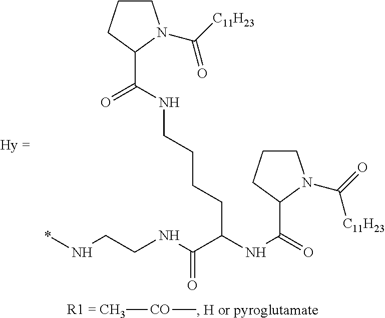 |
| BB6 | 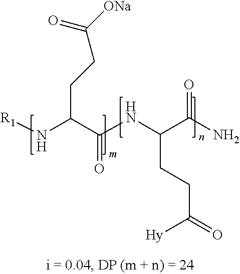 |
| | 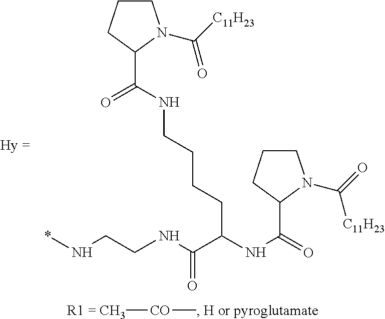 |
| BB7 | 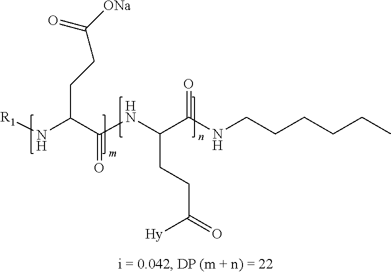 |
| | 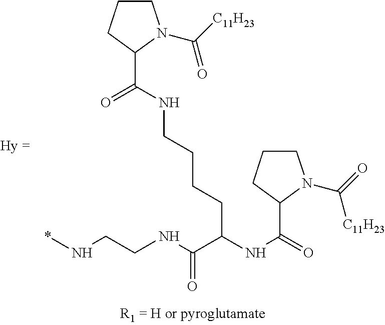 |
| BB8 | 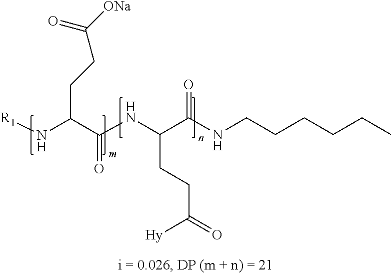 |
| | 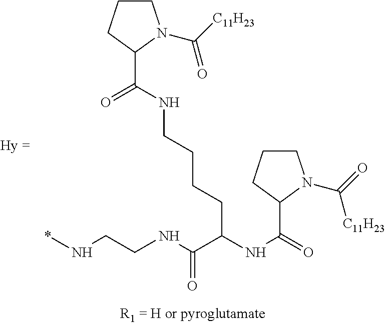 |
| BB9 | 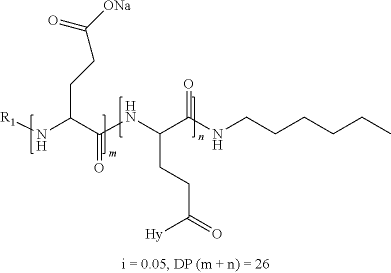 |
| | 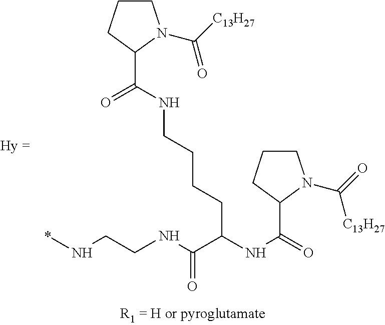 |
| BB10 | 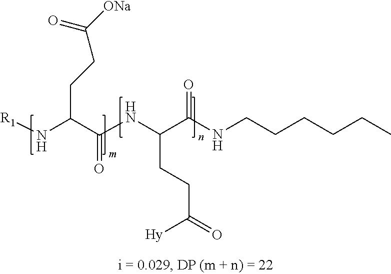 |
| | 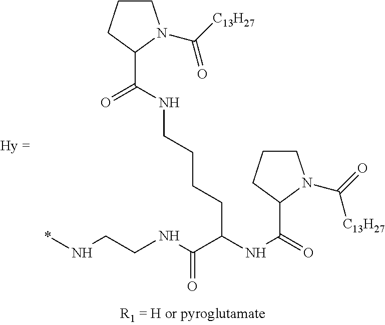 |
| BB11 | 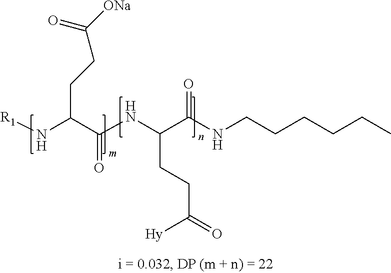 |
| | 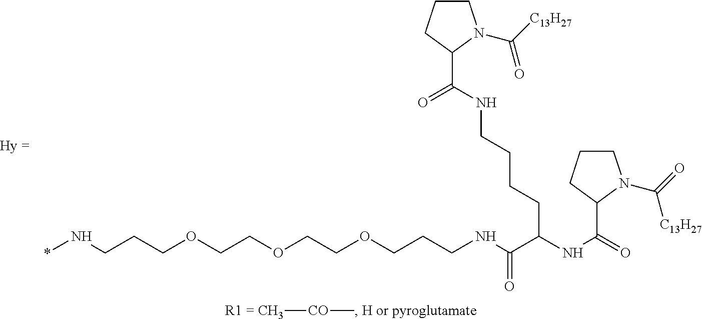 |
| BB12 | 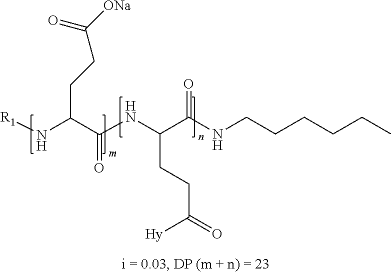 |
| | 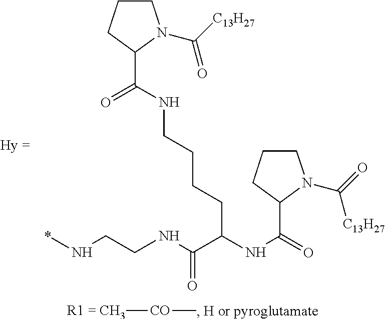 |
| BB13 | 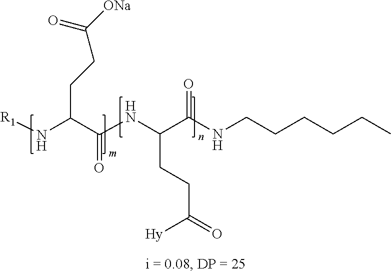 |
| | 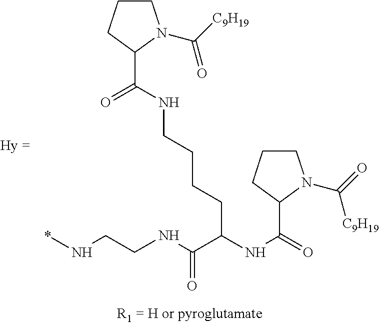 |
| BB19 | 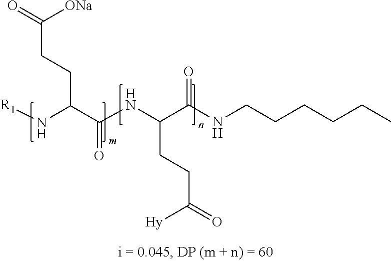 |
| | 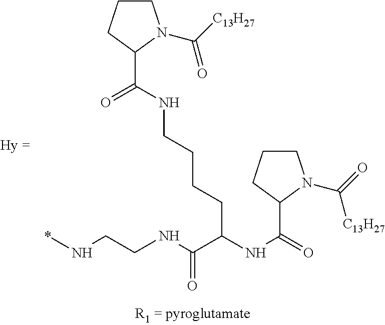 |

### Co-Polyamino Acids According to Formula VII or VIIb

TABLE 1f
List of co-polyamino acids synthesized according to the invention.

| No | CO-POLYAMINOACIDES BEARING CARBOXYLATE LOADS AND HYDROPHOBIC RADICALS |
|---|---|
| BB14 | 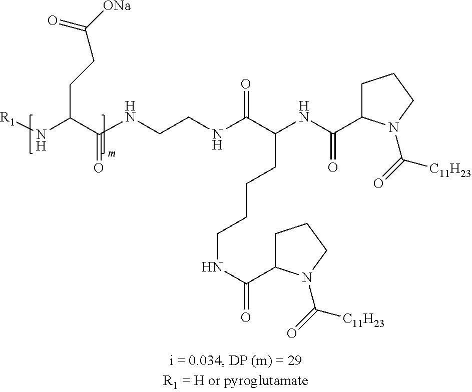 |
| BB15 | 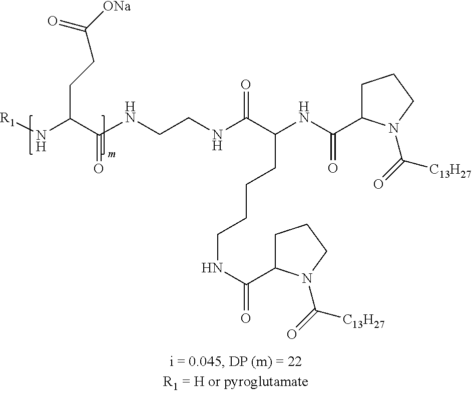 |
| BB16 | 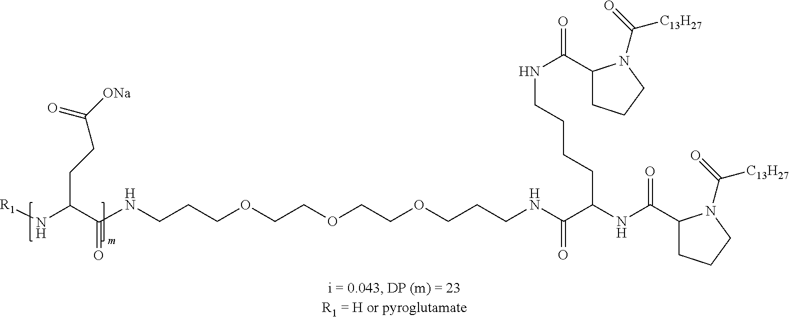 |
| BB17 | 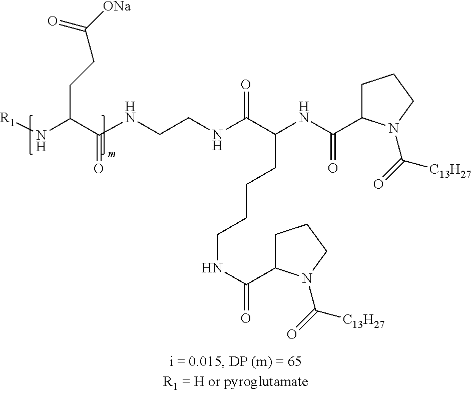 |
| BB18 | 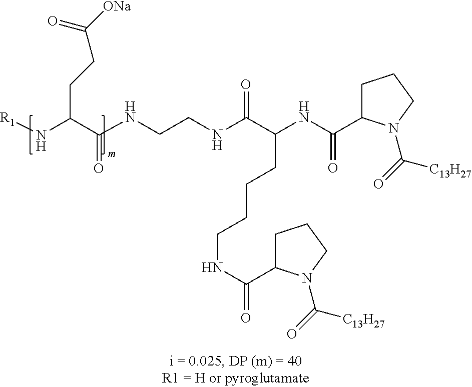 |
| BB20 | 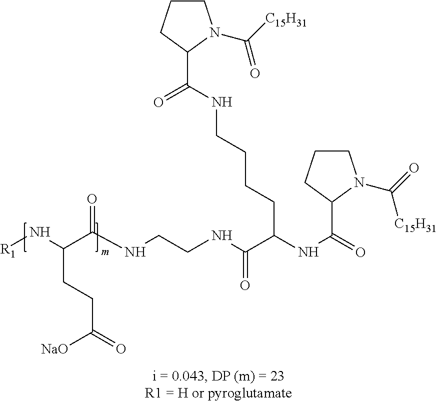 |
| BB21 | 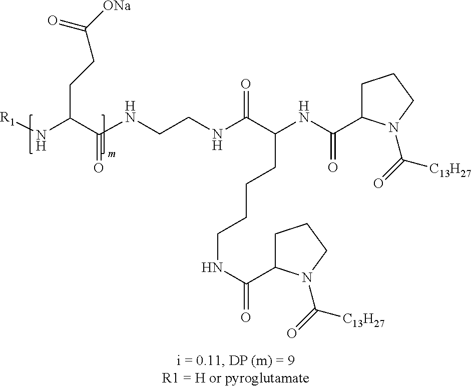 |
| BB22 | 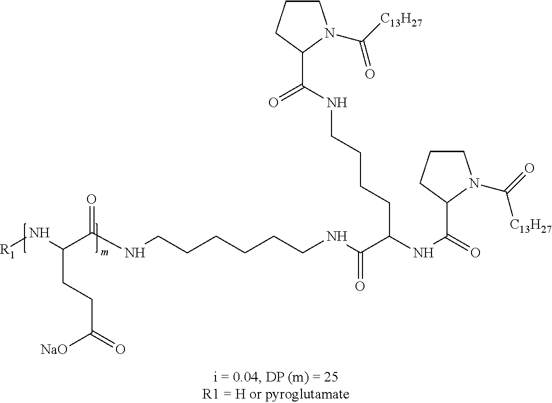 |
| B23 | 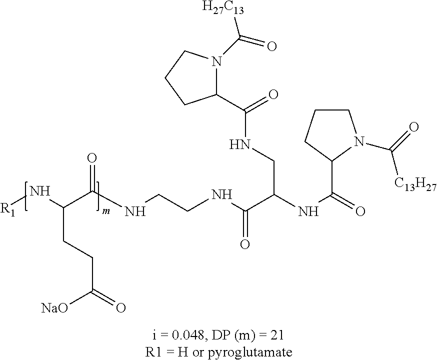 |
| BB24 | 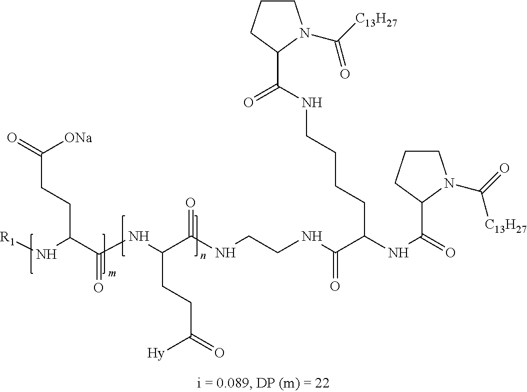 |
| | 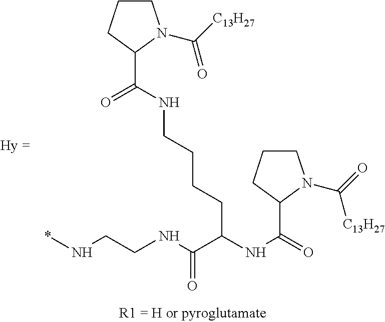 |
| BB25 | 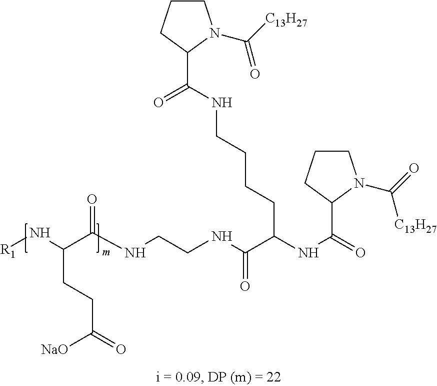 |
| | 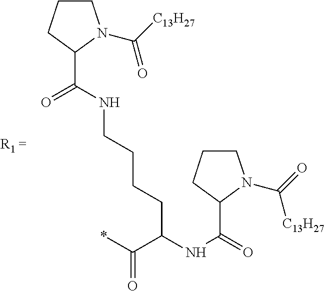 |

### Part BB: Synthesis of Co-Polyamino Acids

### EXAMPLE BB1: CO-POLYAMINO ACID BB1—SODIUM POLY-L-GLUTAMATE MODIFIED BY MOLECULE BA2 AND HAVING A NUMBER-AVERAGE MOLECULAR WEIGHT (MN) OF 2400 G/MOL

Co-Polyamino Acid BB11-1: Poly-L-Glutamic Acid of Number-Average Molar Mass (Mn) 3860 g/Mol from the Polymerization of γ-Benzyl-L-Glutamate N-Carboxyanhydride Initiated by Hexylamine

In a previously oven-dried flask is placed under vacuum γ-benzyl-L-glutamate N-carboxyanhydride (90.0 g, 342 mmol) for 30 minutes, then anhydrous DMF (465 mL) is introduced. The mixture is then stirred under argon until complete dissolution, cooled to 4° C., then hexylamine (1.8 mL 14 mmol) is quickly introduced. The mixture is stirred at 4° C. and room temperature for 2 days. The reaction medium is then heated at 65° C. for 4 hours, cooled to room temperature and then poured dropwise into diisopropyl ether (6 L) with stirring. The white precipitate is recovered by filtration, washed with diisopropyl ether (500×250 mL), then dried under vacuum at 30° C. to give a poly (gamma-benzyl-L-glutamic acid) (PBLG).

A solution of hydrobromic acid (HBr) at 33% in acetic acid (135 mL, 0.77 mol) is added dropwise—to a solution of PBLG (42.1 g) in trifluoroacetic acid (TFA, 325 mL) at 4° C. The mixture is stirred at room temperature for 2 hours, then poured dropwise onto a 1: 1 (v/v) mixture of diisopropyl ether and water with stirring (1.6 L). After stirring for 1 hour 30 minutes, the heterogeneous mixture is allowed to stand overnight. The white precipitate is recovered by filtration, washed with a 1:1 (v/v) mixture of diisopropyl ether and water (200 mL).

The obtained solid is solubilized in water (1 mL) by adjusting the pH to 7 by adding 10 N aqueous sodium hydroxide solution, then 1N aqueous sodium hydroxide solution. After solubilization, the theoretical concentration is adjusted to 25 g/L theoretical by addition of water to obtain a final volume of 1.5 mL.

The solution is filtered through a 0.45 m filter, then purified by ultrafiltration against a solution of NaCl 0.9%, then water until the conductimetry of the permeate is less than 50 μS/cm.

The aqueous solution is then acidified by adding a 37% hydrochloric acid solution until a pH of 2 is reached. After stirring for 4 hours, the precipitate obtained is filtered, then dried under vacuum at 30° C. to give a poly-L-glutamic acid with a number-average molar mass (Mn) of 3860 g/mol relative to a polyoxyethylene standard (PEG).

### Co-Polyamino Acid BB1

Co-polyamino acid BB1-1 (10.0 g) is solubilized in DMF (700 mL) at 30-40° C., then cooled to 0° C. The hydrochloride salt of molecule BA2 (2.95 g, 3.8 mmol) is suspended in DMF (45 mL) and triethylamine (0.39 g, 3.8 mmol) is then added to this suspension and the mixture is slightly heated with stirring until complete dissolution. N-methylmorpholine (NMM, 7.6 g, 75 mmol) in DMF (14 mL) and ethyl chloroformate (ECF, 8.1 g, 75 mmol) are added to a solution of co-polyamino acid at 0° C. After 10 minutes at 0° C., the BA2 molecule solution is added and the medium maintained at 30° C. for 1 hour. The reaction mixture is poured dropwise over 6 L of water containing 15% NaCl weight and HCl (pH 2), and left to stand overnight. The precipitate is recovered by filtration, washed with sodium chloride solution at pH 2 (1 L) and dried under vacuum for about 1 hour. The white solid obtained is taken up in water (600 mL) and the pH is adjusted to 7 by slowly adding a 1 N aqueous solution of NaOH. The volume is adjusted to 700 mL by addition of water. After filtering on a 0.45 μm filter, the clear solution obtained is purified by ultrafiltration against a solution of NaCl 0.9%, then water, until the conductimetry of the permeate is less than 50 μS/cm. After removal, the solution is filtered through a 0.2 m filter and stored at 2-8° C.

Dry extract: 19.7 mg/g
DP (estimated based on RMN ¹H): 23
Based on ¹H NMR: i=0.05
The calculated average molar mass of the co-polyamino acid BB1 is 4350 g/mol.
Aqueous HPLC-SEC (PEG calibrant): Mn=2400 g/mol.

### EXAMPLE BB2: CO-POLYAMINO ACID BB2—SODIUM POLY-L-GLUTAMATE MODIFIED BY MOLECULE BA2 AND HAVING A NUMBER-AVERAGE MOLECULAR WEIGHT (MN) OF 4900 G/MOL

A poly-L-glutamic acid of number-average molecular weight (Mn) 4100 g/mol (5.0 g) obtained by a process similar to that used for the preparation of the co-polyamino acid BB1-1 is solubilized in DMF (205 mL) at 30-40° C. and maintained at this temperature. In parallel, the hydrochloride salt of the BA2 molecule (1.44 g, 1.84 mmol) is suspended in DMF (10 mL) and triethylamine (0.19 g, 1.84 mmol) is added then the mixture is gently heated with stirring until completely dissolved. Solution of molecule BA2 then 2-hydroxypyridine N-oxide (HOPO, 0.31 g, 2.76 mmol) are successively added to the co-polyamino acid solution in DMF, NMM (3.7 g, 36.7 mmol). The reaction medium is then cooled to 0° C., then EDC (0.53 g, 2.76 mmol) is added and the medium is raised to room temperature for 3 hours. The reaction mixture is poured dropwise over 1.55 L of water containing NaCl 15% by weight and HCl (pH 2) with stirring. At the end of the addition, the pH is readjusted to 2 with a N 1 HCl solution, and the suspension is allowed to stand overnight. The precipitate is recovered by filtration, then rinsed with 100 ml of water. The white solid obtained is solubilized in 200 mL of water by slowly adding a 1N aqueous NaOH solution to pH 7 with stirring, then the solution is filtered through a 0.45 μm filter. The clear solution obtained is purified by ultrafiltration against 0.9% NaCl solution, then with water, until the conductimetry of the permeate is less than 50 μS/cm. The obtained solution is filtered through a 0.2 μm filter and stored at 2-8° C.

Dry extract: 16.3 mg/g
DP (estimated based on RMN ¹H): 21
Based on ¹H NMR: i=0.047
The calculated average molar mass of the co-polyamino acid BB2 is 3932 g/mol.
Aqueous HPLC-SEC (PEG calibrant): Mn=4900 g/mol.

### EXAMPLE BB3: CO-POLYAMINO ACID BB3—SODIUM POLY-L-GLUTAMATE MODIFIED BY MOLECULE BA2 AND HAVING A NUMBER-AVERAGE MOLECULAR WEIGHT (MN) OF 6400 G/MOL

Co-Polyamino Acid BB3-1: Poly-L-Glutamic Acid of Number-Average Molecular Weight (Mn) 17500 g/Mol from the Polymerization of γ-Methyl-L-Glutamate N-Carboxyanhydride Triggered by L-Leucinamide

A poly-L-glutamic acid of number-average mass (Mn) 17500 g/mol relative to a standard polymethyl methacrylate (PMMA) is obtained by polymerization of γ-methyl N-carboxyanhydride of glutamic acid using L-leucinamide as an initiator and by deprotecting the methyl esters using a 37% hydrochloric acid solution according to the process described in patent application FR-A-2 801 226.

A sodium poly-L-glutamate acid modified with the molecule BA2 is obtained by a process similar to that used in the preparation of the co-polyamino acid BB2 applied to the hydrochloride salt of molecule BA2 (3.23 g, 4.1 mmol) and co-polyamino acid BB3-1 (11 g).

Dry extract: 27.5 mg/g
DP (estimated based on RMN ¹H): 34
Based on ¹H NMR: i=0.049
The calculated average molar mass of the co-polyamino acid BB3 is 6405 g/mol.
Aqueous HPLC-SEC (PEG calibrant): Mn=6400 g/mol.

### EXAMPLE BB4: CO-POLYAMINO ACID BB4—SODIUM POLY-L-GLUTAMATE MODIFIED BY MOLECULE BA2 AND HAVING A NUMBER-AVERAGE MOLECULAR WEIGHT (MN) OF 10500 G/MOL

A sodium poly-L-glutamate modified with molecule BA2 is obtained by a process similar to that used in the preparation of the co-polyamino acid BB2 applied to the hydrochloride salt of molecule BA2 (5 g, 6.35 mmol) and to a poly-L-glutamic acid of number-average molecular weight Mn=10800 g/mol (21.7 g) obtained by a process similar to that used in the preparation of co-polyamino acid BB11-1.

Dry extract: 28.2 mg/g
DP (estimated based on RMN ¹H): 65
Based on ¹H NMR: i=0.04
The calculated average molar mass of the co-polyamino acid BB4 is 11721 g/mol.
Aqueous HPLC-SEC (PEG calibrant): Mn=10500 g/mol.

### EXAMPLE BB5: CO-POLYAMINO ACID BB5—SODIUM POLY-L-GLUTAMATE CAPPED AT ONE OF ITS ENDS BY AN ACETYL GROUP AND MODIFIED BY MOLECULE BA2 AND HAVING A NUMBER-AVERAGE MOLECULAR WEIGHT (MN) OF 3600 G/MOL

Co-Polyamino Acid B5-1 Poly-L-Glutamic Acid of Mn 3700 g/Mol from the Polymerization of γ-Benzyl-L-Glutamate N-Carboxyanhydride Initiated by Hexylamine and Capped at One End by an Acetyl Group

In a previously oven-dried flask is placed under vacuum γ-benzyl-L-glutamate N-carboxyanhydride (100.0 g, 380 mmol) for 30 minutes then anhydrous DMF (250 mL) is introduced. The mixture is then stirred under argon until complete dissolution, cooled to 4° C., then hexylamine (2.3 mL 17 mmol) is quickly introduced. The mixture is stirred between 4° C. and room temperature for 2 days, then precipitated in diisopropyl ether (3.4 L). The precipitate is collected by filtration, washed twice with diisopropyl ether (225 mL), then dried to give a white solid which is dissolved in 450 mL of THF. N,N-diisopropylethylamine (DIPEA, 31 mL, 176 mmol), then acetic anhydride (17 mL, 176 mmol) are successively added to this solution. After stirring overnight at room temperature, the solution is poured slowly into diisopropyl ether (3 L) over a period of 30 minutes with stirring. After stirring for 1 hour, the precipitate is filtered off, washed twice with diisopropyl ether (200 mL) and then dried under vacuum at 30° C. to give a poly (γ-benzyl-L-glutamic acid) capped at one end by an acetyl group.

A solution of hydrobromic acid (HBr) at 33% in acetic acid (235 mL, 1.34 mol) is added dropwise—to a solution of co-polyamino acid capped (72 g) in trifluoroacetic acid (TFA, 335 mL) at 4° C. The mixture is stirred at room temperature for 3 hours 30 minutes and then poured dropwise onto a 1:1 (v/v) mixture of diisopropyl ether and water with stirring (4 L). After stirring for 2 hours, the heterogeneous mixture is allowed to stand overnight. The white precipitate is recovered by filtration, washed with a 1:1 (v/v) mixture of diisopropyl ether and water (340 mL), then with water (340 mL). The obtained solid is then solubilized in water (1.5 L) by adjusting the pH to 7 by adding 10 aqueous solution of sodium hydroxide, then a 1N aqueous sodium hydroxide solution. After solubilization, the theoretical concentration is adjusted to 20 g/L theoretical by adding water to obtain a final volume of 2.1 L. The solution is filtered through a 0.45 μm filter and then purified by ultrafiltration against a solution of NaCl 0.9%, and then water until the conductimetry of the permeate is less than 50 μS/cm. The co-polyamino acid solution is then concentrated until a final volume of 1.8 L. The aqueous solution is then acidified by adding 37% hydrochloric acid solution until a pH of 2 is reached. After stirring for 4 hours, the precipitate obtained is filtered, washed with water (330 mL) and then dried under vacuum at 30° C. to give a poly-L-glutamic acid of number-average molar mass (Mn) 3700 g/mol relative to a standard of polyoxyethylene (PEG).

### Co-Polyamino Acid BB5

By a process similar to that used in the preparation of co-polyamino acid BB2 applied to the hydrochloride salt of molecule BA2 (6.92 g, 8.8 mmol) and co-polyamino acid BB5-1 (30.0 g), a sodium poly-L-glutamate capped at one end by an acetyl group and modified by molecule BA2 is obtained.

Dry extract: 29.4 mg/g
DP (estimated based on RMN ¹H): 23
Based on ¹H NMR: i=0.042
The calculated average molar mass of the co-polyamino acid BB5 is 4302 g/mol.
Aqueous HPLC-SEC (PEG calibrant): Mn=3600 g/mol.

### EXAMPLE BB6: CO-POLYAMINO ACID BB6—SODIUM POLY-L-GLUTAMATE CAPPED AT ONE OF ITS ENDS BY AN ACETYL GROUP AND MODIFIED BY MOLECULE BA2 AND HAVING A NUMBER-AVERAGE MOLECULAR WEIGHT (MN) OF 4100 G/MOL

A sodium poly-L-glutamate capped at one end by an acetyl group and modified by molecule BA2 is obtained by a process similar to that used in the preparation of the co-polyamino acid BB2 applied to the hydrochloride salt of molecule BA2 (5.8 g, 7.4 mmol) and to a poly-L-glutamic acid of number-average molecular weight Mn=3800 g/mol (25 g) obtained by a process similar to that used in the preparation of co-polyamino acid BB5-1 using ammonia instead of hexylamine.

Dry extract: 27.6 mg/g
DP (estimated based on RMN ¹H): 24
Based on ¹H NMR: i=0.04
The calculated average molar mass of the co-polyamino acid BB6 is 4387 g/mol.
Aqueous HPLC-SEC (PEG calibrant): Mn=4100 g/mol.

### EXAMPLE BB7: CO-POLYAMINO ACID BB7—SODIUM POLY-L-GLUTAMATE MODIFIED BY MOLECULE BA2 AND HAVING A NUMBER-AVERAGE MOLECULAR WEIGHT (MN) OF 4200 G/MOL

A sodium poly-L-glutamate modified with molecule BA2 is obtained by a process similar to that used in the preparation of the co-polyamino acid BB2 applied to the hydrochloride salt of molecule BA2 (7.07 g, 9.0 mmol) and to a poly-L-glutamic acid of number-average molecular weight Mn=3600 g/mol (30.0 g) obtained by a process similar to that used in the preparation of co-polyamino acid BB1-1.

Dry extract: 28.3 mg/g
DP (estimated based on RMN ¹H): 22
Based on ¹H NMR: i=0.042
The calculated average molar mass of the co-polyamino acid BB7 is 4039 g/mol.
Aqueous HPLC-SEC (PEG calibrant): Mn=4200 g/mol.

### EXAMPLE BB8: CO-POLYAMINO ACID BB8—SODIUM POLY-L-GLUTAMATE MODIFIED BY MOLECULE BA2 AND HAVING A NUMBER-AVERAGE MOLECULAR WEIGHT (MN) OF 5200 G/MOL

A sodium poly-L-glutamate modified with molecule BA2 is obtained by a process similar to that used in the preparation of the co-polyamino acid BB2 applied to the hydrochloride salt of molecule BA2 (0.85 g, 1.1 mmol) and to a poly-L-glutamic acid of number-average molecular weight Mn=4100 g/mol (5.0 g) obtained by a process similar to that used in the preparation of co-polyamino acid BB1-1.

Dry extract: 28.6 mg/g
DP (estimated based on RMN ¹H): 21
Based on ¹H NMR: i=0.026
The calculated average molar mass of the co-polyamino acid BB8 is 3620 g/mol.
Aqueous HPLC-SEC (PEG calibrant): Mn=5200 g/mol.

### EXAMPLE BB9: CO-POLYAMINO ACID BB9—SODIUM POLY-L-GLUTAMATE MODIFIED BY MOLECULE BA3 AND HAVING A NUMBER-AVERAGE MOLECULAR WEIGHT (MN) OF 4700 G/MOL

A sodium poly-L-glutamate modified with molecule BA3 is obtained by a process similar to that used in the preparation of the co-polyamino acid BB2 applied to the hydrochloride salt of molecule BA3 (3.05 g, 3.6 mmol) and to a poly-L-glutamic acid of number-average molecular weight Mn=4100 g/mol (10.0 g) obtained by a process similar to that used in the preparation of co-polyamino acid BB1-1.

Dry extract: 28.6 mg/g
DP (estimated based on RMN ¹H): 26
Based on ¹H NMR: i=0.05
The calculated average molar mass of the co-polyamino acid BB9 is 4982 g/mol.
Aqueous HPLC-SEC (PEG calibrant): Mn=4700 g/mol.

### EXAMPLE BB10: CO-POLYAMINO ACID BB10—SODIUM POLY-L-GLUTAMATE MODIFIED BY MOLECULE BA3 AND HAVING A NUMBER-AVERAGE MOLECULAR WEIGHT (MN) OF 4200 G/MOL

A sodium poly-L-glutamate modified with molecule BA3 is obtained by a process similar to that used in the preparation of the co-polyamino acid BB2 applied to the hydrochloride salt of molecule BA3 (1.90 g, 2.3 mmol) and to a poly-L-glutamic acid of number-average molecular weight Mn=3500 g/mol (10.0 g) obtained by a process similar to that used in the preparation of co-polyamino acid BB1-1.

Dry extract: 25.9 mg/g
DP (estimated based on RMN ¹H): 22
Based on ¹H NMR: i=0.029
The calculated average molar mass of the co-polyamino acid BB10 is 3872 g/mol.
Aqueous HPLC-SEC (PEG calibrant): Mn=4200 g/mol.

### EXAMPLE BB11: CO-POLYAMINO ACID BB1—SODIUM POLY-L-GLUTAMATE CAPPED AT ONE OF ITS ENDS BY AN ACETYL GROUP AND MODIFIED BY MOLECULE BA4 AND HAVING A NUMBER AVERAGE MOLECULAR WEIGHT (MN) OF 3900 G/MOL

A sodium poly-L-glutamate capped at one end by an acetyl group and modified by molecule BA4 is obtained by a process similar to that used in the preparation of the co-polyamino acid BB2 applied to the hydrochloride salt of molecule BA4 (2.21 g, 2.2 mmol) and to a poly-L-glutamic acid of number-average molecular weight Mn=3700 g/mol (10 g) obtained by a process similar to that used in the preparation of co-polyamino acid BB5-1 using ammonia instead of hexylamine.

Dry extract: 28.1 mg/g
DP (estimated based on RMN ¹H): 22
Based on ¹H NMR: i=0.032
The calculated average molar mass of the co-polyamino acid BB111 is 4118 g/mol.
Aqueous HPLC-SEC (PEG calibrant): Mn=3900 g/mol.

### EXAMPLE BB12: CO-POLYAMINO ACID BB12—SODIUM POLY-L-GLUTAMATE CAPPED AT ONE OF ITS ENDS BY AN ACETYL GROUP AND MODIFIED BY MOLECULE BA3 AND HAVING A NUMBER AVERAGE MOLECULAR WEIGHT (MN) OF 3900 G/MOL

A sodium poly-L-glutamate capped at one end by an acetyl group and modified by molecule BA3 is obtained by a process similar to that used in the preparation of the co-polyamino acid BB2 applied to the hydrochloride salt of molecule BA3 (1.9 g, 2.3 mmol) and to a poly-L-glutamic acid of number-average molecular weight Mn=3600 g/mol (10 g) obtained by a process similar to that used in the preparation of co-polyamino acid BB5-1 using ammonia instead of hexylamine.

Dry extract: 26.7 mg/g
DP (estimated based on RMN ¹H): 23
Based on ¹H NMR: i=0.03
The calculated average molar mass of the co-polyamino acid BB12 is 4145 g/mol.
Aqueous HPLC-SEC (PEG calibrant): Mn=3900 g/mol.

### EXAMPLE BB13: CO-POLYAMINO ACID BB13—SODIUM POLY-L-GLUTAMATE MODIFIED BY MOLECULE BA1 AND HAVING A NUMBER-AVERAGE MOLECULAR WEIGHT (MN) OF 2800 G/MOL

A sodium poly-L-glutamate modified with molecule BA1 is obtained by a process similar to that used in the preparation of the co-polyamino acid BB1 applied to the hydrochloride salt of molecule BA (3.65 g, 5 mmol) and to a poly-L-glutamic acid of number-average molecular weight Mn=3600 g/mol (10 g) obtained by a process similar to that used in the preparation of co-polyamino acid BB1-1.

Dry extract: 25.6 mg/g
DP (estimated based on RMN ¹H): 25
Based on ¹H NMR: i=0.08
The calculated average molar mass of the co-polyamino acid BB13 is 5253 g/mol.
Aqueous HPLC-SEC (PEG calibrant): Mn=2800 g/mol.

### EXAMPLE BB19: CO-POLYAMINO ACID BB19—SODIUM POLY-L-GLUTAMATE MODIFIED BY MOLECULE BA3 AND HAVING A NUMBER-AVERAGE MOLECULAR WEIGHT (MN) OF 7700 G/MOL

A sodium poly-L-glutamate modified by molecule BA3 is obtained by a process similar to that used in the preparation of co-polyamino acid AB23 applied to the hydrochloride salt of molecule BA3 and co-polyamino acid AB23-1.

Dry extract: 25.3 mg/g
DP (estimated based on RMN ¹H): 60
Based on ¹H NMR: i=0.045
The calculated average molar mass of the co-polyamino acid BB19 is 11188 g/mol.
Organic HPLC-SEC (PEG Calibrator): Mn=7700 g/mol.

### EXAMPLE BB14: CO-POLYAMINO ACID BB14—SODIUM POLY-L-GLUTAMATE MODIFIED AT ONE OF ITS ENDS BY MOLECULE BA2 AND HAVING A NUMBER AVERAGE MOLECULAR WEIGHT (MN) OF 4020 G/MOL

Hydrochloride salt of molecule BA2 (2.12 g, 2.70 mmol), chloroform (40 mL), molecular sieve 4 Å (1.5 g), as well as Amberlite IRN 150 ion exchange resin (1.5 g) are successively added to a suitable container. After 1 hour of stirring on rollers, the medium is filtered and the resin is rinsed with chloroform. The mixture is evaporated, then co-evaporated with toluene. The residue is solubilized in anhydrous DMF (20 mL) for direct use in the polymerization reaction.

γ-benzyl-L-glutamate N-carboxyanhydride (18 g, 68.42 mmol) is placed under vacuum for 30 minutes in an oven-dried flask and then anhydrous DMF (100 mL) is added. The mixture is stirred under argon until complete solubilization, cooled to 4° C., then the solution of molecule BA2 prepared as described above is rapidly introduced. The mixture is stirred at 4° C. and room temperature for 2 days, then heated at 65° C. for 2 hours. The reaction mixture is then cooled to room temperature, then poured dropwise into diisopropyl ether (1.2 L) with stirring. The white precipitate is recovered by filtration, washed twice with diisopropyl ether (100 mL), then dried under vacuum at 30° C. to obtain a white solid. The solid is diluted in TFA (105 mL), and a solution of 33% hydrobromic acid (HBr) in acetic acid (38 mL, 220 mmol) is then added dropwise—at 0° C. The solution is stirred for 2 hours at room temperature and is then poured dropwise on a mixture of 1:1 (v/v) diisopropyl ether/water and with stirring (600 mL). After stirring for 2 hours, the heterogeneous mixture is allowed to stand overnight. The white precipitate is recovered by filtration, washed successively with a mixture of 1:1 (v/v) diisopropyl ether and water (200 mL) and then with water (100 mL). The obtained solid is solubilized in water (450 mL) by adjusting the pH to 7 by adding 10 N aqueous sodium hydroxide solution, then 1 N aqueous sodium hydroxide solution. The mixture is filtered through a 0.45 μm filter, then is purified by ultrafiltration against 0.9% NaCl solution, then water until the conductimetry of the permeate is less than 50 μS/cm. The co-polyamino acid solution is then concentrated to about 30 g/L theoretical and the pH is adjusted to 7.0. The aqueous solution is filtered through 0.2 μm and stored at 4° C.

Dry extract: 22.3 mg/g
DP (estimated by ¹H) NMR=29 where i=0.034
The calculated average molar mass of the co-polyamino acid BB14 is 5089 g/mol.
Aqueous HPLC-SEC (PEG calibrant): Mn=4020 g/mol.

### EXAMPLE BB15: CO-POLYAMINO ACID BB15—SODIUM POLY-L-GLUTAMATE MODIFIED AT ONE OF ITS ENDS BY MOLECULE BA3 AND HAVING A NUMBER AVERAGE MOLECULAR WEIGHT (MN) OF 3389 G/MOL

By a process similar to that used in the preparation of co-polyamino acid BB14 applied to the hydrochloride salt of molecule BA3 (3.62 g, 4.32 mmol) and 25.0 g (94.97 mmol) of γ-benzyl-L-glutamate N-carboxyanhydride, a sodium poly-L-glutamate modified at one end by molecule BA3 is obtained.

Dry extract: 30.4 mg/g
DP (estimated by ¹H) NMR=24 where i=0.042
The calculated average molar mass of the co-polyamino acid BB15 is 4390 g/mol.
Aqueous HPLC-SEC (PEG calibrant): Mn=3389 g/mol.

### EXAMPLE BB16: CO-POLYAMINO ACID BB16—SODIUM POLY-L-GLUTAMATE MODIFIED AT ONE OF ITS ENDS BY MOLECULE BA4 AND HAVING A NUMBER AVERAGE MOLECULAR WEIGHT (MN) OF 3300 G/MOL

By a process similar to that used in the preparation of co-polyamino acid BB14 applied to the hydrochloride salt of molecule BA4 (5.70 g, 5.70 mmol) and 29.99 g (113.9 mmol) of γ-benzyl-L-glutamate N-carboxyanhydride, a sodium poly-L-glutamate modified at one end by molecule BA4 is obtained.

Dry extract: 32.3 mg/g
DP (estimated by ¹H) NMR=23 where i=0.043
The calculated average molar mass of the co-polyamino acid BB16 is 4399 g/mol.
Aqueous HPLC-SEC (PEG calibrant): Mn=3300 g/mol.

### EXAMPLE BB17: CO-POLYAMINO ACID BB17—SODIUM POLY-L-GLUTAMATE MODIFIED AT ONE OF ITS ENDS BY MOLECULE BA3 AND HAVING A NUMBER AVERAGE MOLECULAR WEIGHT OF 10700 G/MOL

By a process similar to that used in the preparation of co-polyamino acid BB14 applied to the hydrochloride salt of molecule BA3 (2.51 g, 3 mmol) and 52.7 g (200 mmol) of γ-benzyl-L-glutamate N-carboxyanhydride, a sodium poly-L-glutamate modified at one end by molecule BA3 is obtained.

Dry extract: 24.5 mg/g
DP (estimated by ¹H) NMR=65 where i=0.015
The calculated average molar mass of the co-polyamino acid BB17 is 10585 g/mol.
Aqueous HPLC-SEC (PEG calibrant): Mn=10700 g/mol.

### EXAMPLE BB18: CO-POLYAMINO ACID BB18—SODIUM POLY-L-GLUTAMATE MODIFIED AT ONE OF ITS ENDS BY MOLECULE BA3 AND HAVING A NUMBER AVERAGE MOLECULAR WEIGHT OF 6600 G/MOL

By a process similar to that used in the preparation of co-polyamino acid BB14 applied to the hydrochloride salt of molecule BA3 (2.51 g, 3 mmol) and 31.6 g (120 mmol) of γ-benzyl-L-glutamate N-carboxyanhydride, a sodium poly-L-glutamate modified at one end by molecule BA3 is obtained.

Dry extract: 27.3 mg/g
DP (estimated by ¹H) NMR=40 where i=0.025
The calculated average molar mass of the co-polyamino acid BB118 is 6889 g/mol.
Aqueous HPLC-SEC (PEG calibrant): Mn=6600 g/mol.

### EXAMPLE BB20: CO-POLYAMINO ACID BB20—SODIUM POLY-L-GLUTAMATE MODIFIED AT ONE OF ITS ENDS BY MOLECULE BA5 AND HAVING A NUMBER AVERAGE MOLECULAR WEIGHT (MN) OF 2800 G/MOL

A sodium poly-L-glutamate modified at one end by molecule BA5 is obtained by a process similar to that used in the preparation of the co-polyamino acid BB14 applied to molecule BA5 in the form of free amine (1.70 g, 1.98 mmol) and γ-benzyl-L-glutamate N-carboxyanhydride (11.46 g, 43.5 mmol).

Dry extract: 20.7 mg/g
DP (estimated by ¹H) NMR=23 where i=0.043
The calculated average molar mass of the co-polyamino acid BB20 is 4295 g/mol.
Aqueous HPLC-SEC (PEG calibrant): Mn=2800 g/mol.

### EXAMPLE BB21: CO-POLYAMINO ACID BB21—SODIUM POLY-L-GLUTAMATE MODIFIED AT ONE OF ITS ENDS BY MOLECULE BA3 AND HAVING A NUMBER AVERAGE MOLECULAR WEIGHT (MN) OF 1100 G/MOL

A sodium poly-L-glutamate modified at one end by molecule BA3 is obtained by a process similar to that used in the preparation of the co-polyamino acid BB14 applied to molecule BA3 in the form of free amine (3.814 g, 4.75 mmol) and γ-benzyl-L-glutamate N-carboxyanhydride (10.0 g, 38.0 mmol).

Dry extract: 16.1 mg/g
DP (estimated by ¹H) NMR=9 where i=0.11
The calculated average molar mass of the co-polyamino acid BB21 is 2123 g/mol.
Aqueous HPLC-SEC (PEG calibrant): Mn=1100 g/mol.

### EXAMPLE BB22: CO-POLYAMINO ACID BB22—SODIUM POLY-L-GLUTAMATE MODIFIED AT ONE OF ITS ENDS BY MOLECULE BA6 AND HAVING A NUMBER AVERAGE MOLECULAR WEIGHT (MN) OF 3300 G/MOL

A sodium poly-L-glutamate modified at one end by molecule BA6 is obtained by a process similar to that used in the preparation of co-polyamino acid BB14 applied to molecule BA6 as free amine (4.45 g, 5.18 mmol) and 30.0 g (113.96 mmol) of γ-benzyl-L-glutamate N-carboxyanhydride.

Dry extract: 29.0 mg/g
DP (estimated by ¹H) NMR=25 where i=0.04
The calculated average molar mass of the co-polyamino acid BB22 is 4597 g/mol.
Aqueous HPLC-SEC (PEG calibrant): Mn=3300 g/mol.

### EXAMPLE BB23: CO-POLYAMINO ACID BB23—SODIUM POLY-L-GLUTAMATE MODIFIED AT ONE OF ITS ENDS BY THE MOLECULE BA7 AND HAVING A NUMBER-AVERAGE MOLECULAR WEIGHT (MN) OF 2900 G/MOL

A sodium poly-L-glutamate modified at one end by molecule BA7 is obtained by a process similar to that used in the preparation of the co-polyamino acid BB14 applied to molecule BA7 in the form of free amine (3.05 g, 4.01 mmol) and γ-benzyl-L-glutamate N-carboxyanhydride (22.78 g, 86.5 mmol).

Dry extract: 16.9 mg/g
DP (estimated by ¹H) NMR=21 where i=0.048
The calculated average molar mass of the co-polyamino acid BB23 is 3894 g/mol.
Aqueous HPLC-SEC (PEG calibrant): Mn=2900 g/mol.

### EXAMPLE BB24: CO-POLYAMINO ACID BB24—SODIUM POLY-L-GLUTAMATE MODIFIED AT ONE END BY MOLECULE BA3 AND MODIFIED BY MOLECULE BA3 AND HAVING A NUMBER-AVERAGE MOLECULAR WEIGHT (MN) OF 2300 G/MOL

### Co-Polyamino Acid BB24-1: Poly-L-Glutamic Acid Modified at One End by Molecule BA3 and Capped at the Other End by Pidolic Acid.

γ-benzyl-L-glutamate N-carboxyanhydride (122.58 g, 466 mmol) is placed under vacuum for 30 minutes in an oven-dried flask and then anhydrous DMF (220 mL) is added. The mixture is stirred under argon until complete solubilization, cooled to 10° C., then a solution of molecule BA3 in the form of free amine (17.08 g, 21.3 mmol) in chloroform (40 ml) is quickly introduced. The mixture is stirred at 0° C. and room temperature for 2 days, then heated at 65° C. for 4 hours. The reaction mixture is then cooled to 25° C., then pidolic acid (13.66 g, 105.8 mmol) is added, HOBt (2.35 g, 15.3 mmol) and EDC (20.28 g, 105.8 mmol) are added. After 24 hours of stirring at 25° C., the solution is concentrated under vacuum to eliminate chloroform and 50% of DMF. The reaction mixture is then heated to 55° C. and 1150 mL of methanol is added after 1 hour. The reaction mixture is then cooled to 0° C. After 18 hours, the white precipitate is recovered by filtration, washed three times with 270 mL of diisopropyl ether, then dried under vacuum at 30° C. to obtain a white solid. The solid is diluted in TFA (390 mL), and a solution of 33% hydrobromic acid (HBr) in acetic acid (271 mL, 1547 mmol) is then added dropwise—at 0° C. The solution is stirred for 2 hours at room temperature and is then poured dropwise on a mixture of 1:1 (v/v) diisopropyl ether/water and with stirring (970 mL). After stirring for 2 hours, the heterogeneous mixture is allowed to stand overnight. The white precipitate is recovered by filtration, washed successively with diisopropyl ether (380 mL), then twice with water (380 mL). The obtained solid is solubilized in water (3.6 L) by adjusting the pH to 7 by adding a 10N aqueous solution of sodium hydroxide, then a 1N aqueous sodium hydroxide solution. The mixture is filtered through a 0.45 μm filter, then purified by ultrafiltration against 0.9% NaCl solution, 0.1N NaOH solution, 0.9% NaCl solution, phosphate buffer solution (150 mM), a solution of NaCl 0.9% then water until the conductimetry of the permeate is below 50 μS/cm. The co-polyamino acid solution is then concentrated to about 30 g/L theoretical, filtered on 0.2 microns and acidified to pH 2 with stirring by addition of a solution of HCl 37%. The precipitate is then recovered by filtration, washed twice with water, then dried under vacuum at 30° C. to obtain a white solid.

### Co-Polyamino Acid BB24

A sodium poly-L-glutamate modified at one end by molecule BA3 and modified by molecule BA3 is obtained by a process similar to that used in the preparation of co-polyamino acid BB2 applied to molecule BA3 as free amine (1.206 g, 1.50 mmol) and co-polyamino acid BB24-1 (5.5 g, 33.4 mmol).

Dry extract: 19.0 mg/g
DP (estimated based on RMN ¹H): 22
Based on ¹H NMR: i=0.089
The calculated average molar mass of the co-polyamino acid BB24 is 4826 g/mol.
Aqueous HPLC-SEC (PEG calibrant): Mn=2300 g/mol.

### EXAMPLE BB25: SODIUM CO-POLYAMINO ACID BB25—POLY-L-GLUTAMATE MODIFIED AT ONE END BY MOLECULE BA3 AND ON THE OTHER END BY MOLECULE B8 AND HAVING A NUMBER-AVERAGE MOLECULAR WEIGHT (MN) OF 2000 G/MOL

DCC (0.257 g, 1.24 mmol) and NHS (0.143 g, 1.24 mmol) are added to a solution of molecule B8 (0.946 g, 1.24 mmol) in DMF (8 mL). After stirring for 16 hours at room temperature, the solution is filtered to be used directly in the next reaction.

γ-benzyl-L-glutamate N-carboxyanhydride (6.0 g, 22.8 mmol) is placed under vacuum for 30 minutes in an oven-dried flask and then anhydrous DMF (14 mL) is added. The mixture is then stirred under argon until complete dissolution is complete, cooled to 0° C., then a solution of molecule BA3 in the form of a free amine (0.832 g, 1.04 mmol) in chloroform (2.0 mL) is quickly introduced. After 18 hours of stirring at 0° C., the previously prepared solution of molecule B8 is added. The solution is stirred—at between 0° C. and room temperature for 22 hours, then poured dropwise into diisopropylether (0.34 L) with stirring. The white precipitate is recovered by filtration, washed with diisopropyl ether (7×15 mL), then dried under reduced pressure at 30° C. to give a white solid. The solid is diluted in TFA (23 mL), then the solution is cooled to 4° C. A solution of HBr at 33% in acetic acid (15 mL, 85.7 mmol) is then added dropwise. The mixture is stirred at room temperature for 2 hours, then poured dropwise onto a 1:1 (v/v) mixture of diisopropyl ether and water with stirring (0.28 L). After stirring for 2 hours, the heterogeneous mixture is allowed to stand overnight. The white precipitate is recovered by filtration, washed twice with a 1:1 (v/v) mixture of diisopropyl ether and water (24 mL), then twice with water (24 mL). The obtained solid is then solubilized in water (0.16 L) by adjusting the pH to 12 by adding a 10N aqueous sodium hydroxide solution, then a 1N aqueous sodium hydroxide solution. After 30 minutes the pH is adjusted to 7 by slowly adding an aqueous solution of 1N HCl. The solution is filtered through a 0.45 μm filter, then purified by ultrafiltration against a solution of NaCl 0.9%, then water until the Permeate conductimetry is less than 50 μS/cm. The obtained solution is filtered through a 0.2 μm filter and stored at 2-8° C.

Dry extract: 18.9 mg/g
DP (estimated based on RMN ¹H): 22
Based on ¹H NMR: i₁=0.09
The calculated average molar mass of the co-polyamino acid BB25 is 4871 g/mol.
Aqueous HPLC-SEC (PEG calibrant): Mn=2000 g/mol.

### Part C Compositions

### EXAMPLE C1: RAPID INSULIN SOLUTION (HUMALOG®) 100 U/ML

This solution is a commercial insulin lispro solution marketed by ELI LILLY under the name Humalog. This product is a rapid analog insulin. The excipients in Humalog® are metacresol (3.15 mg/mL), glycerol (16 mg/mL), disodium phosphate (1.88 mg/mL), zinc oxide (to produce 0.0197 mg of zinc ion/mL), sodium hydroxide and hydrochloric acid for pH adjustment (pH 7-7.8) and water.

### EXAMPLE C2: RAPID INSULIN LISPRO SOLUTION—600 U/ML

This solution is an insulin solution prepared from insulin lispro powder. This product is a rapid analog insulin. The excipients used are meta-cresol, glycerol, zinc oxide, sodium hydroxide and hydrochloric acid for pH adjustment (pH 7-7.8) and water. The concentration of zinc oxide is 1800 μM and that of glycerol is 230 mM. The concentration of m-cresol varies according to the desired concentrations in the final preparations.

### EXAMPLE C3: BASAL ANALOG INSULIN SOLUTION (LANTUS®)—100 U/ML

This solution is a commercial insulin glargine solution marketed by SANOFI under the name Lantus®. This product is a basal analog insulin. Excipients of Lantus® are zinc chloride (460 μM), metacresol (2.7 mg/mL), glycerol (85%) (20 mg/mL), Tween 20 (16 μM), sodium hydroxide and hydrochloric acid for pH adjustment (pH 4) and water.

### EXAMPLE C4: GLARGINE INSULIN SOLUTION—100-400 U/ML

This solution is an insulin glargine solution prepared from insulin glargine powder. This product is a slow analog insulin. The excipients used are zinc chloride, metacresol, glycerol, sodium hydroxide and hydrochloric acid for pH adjustment (pH 4) and water. The concentration of zinc is 4.5 μM for 1 IU/ml of insulin. The concentration of glycerol and phenol excipients m-cresol and phenol vary according to the desired concentrations in the final preparations.

### EXAMPLE CA: DETERMINATION OF MINIMUM RATIOS TO SOLUBILIZE INSULIN GLARGINE

Protocol to Determine the Minimum Concentration to Solubilize 50 U/mL Insulin Glargine with a pH of 7.1.

Concentrated solutions of zinc chloride, sodium chloride, m-cresol and glycerin are added to a stock solution of co-polyamino acid with a pH of 7.2±0.3. 0.5 mL of an insulin glargine solution at a concentration of 100 U/mL, prepared according to example C3 or C4, is added to a volume of 0.5 mL of the co-polyamino acid solution to obtain a 50 U/mL co-polyamino acid/insulin glargine composition. The quantity of excipients added is selected in order to obtain a concentration of 0-1 mM zinc chloride, 0-10 mM sodium chloride, 35 Mm-cresol and 184 mM in the 50 U/mL co-polyamino acid/insulin glargine composition. The concentration of co-polyamino acid varies from one preparation to another: solutions having co-polyamino acid concentrations varying by no more than 0.25 mg/ml are prepared in this way.

Following the addition of the glargine solution, a turbidity appears. The pH is adjusted to pH 7.1 by adding concentrated NaOH and the solution is placed in an oven at 40° C. overnight. After the night at 40° C. the samples are visually inspected and subjected to a static light scattering measurement at a 173° angle using a zetasizer (Malvern). The minimum concentration of polyamino acid required to solubilize insulin glargine is defined as the lowest concentration at which the co-polyamino acid/insulin glargine with a pH of 7.1±0.1 is visually clear, does not contain any visible particles and has a diffused intensity of less than 1500 kcps.

TABLE 1
Minimum ratio for solubilizing insulin glargine.

| | | | | Concentration | |
|---|---|---|---|---|---|
| | | | | of co-polyamino | |
| | | | | acid (mg/mL) at | Ratio [Hy]/ |
| | Co- | | | the solubilization | [insulin glargine] |
| | polyamino | [ZnCl₂] | [NaCl] | threshold of 50 U/mL | (mol/mol) at the |
| Composition | acid | (mM) | (mM) | glargine with a pH of 7.1 | solubilization threshold |
| CA1 | AB14 | 0.23 | 0 | 4.6 | 4.6 |
| | | 0.98 | 10 | 2.5 | 2.5 |
| CA2 | AB37 | 0.23 | 0 | 4.25 | 3.8 |
| | | 0.95 | 0 | 2.7 | 2.4 |
| | | 0.77 | 10 | 2.00 | 1.8 |
| CA3 | AB35 | 0.23 | 0 | 1.5 | 1.3 |
| | | 0.23 | 10 | 1 | 0.9 |
| | | 0.42 | 10 | 0.75 | 0.6 |
| | | 0.45 | 5 | 0.87 | 0.7 |
| CA4 | AB33 | 0.23 | 0 | 4 | 3.4 |
| | | 0.76 | 10 | 1.75 | 1.5 |
| CA5 | AB36 | 0.23 | 0 | 4 | 3.3 |
| | | 0.23 | 10 | 1.25 | 1 |
| CA6 | AB24 | 0.23 | 0 | 1.2 | 3 |
| | | 0.23 | 5 | 1.1 | 2.7 |
| | | 0.23 | 10 | 1 | 2.5 |

The addition of zinc alone or in combination with salt reduces the concentration of co-polyamino acid required to solubilize insulin glargine.

### EXAMPLE CB: COMPOSITIONS COMPRISING INSULIN GLARGINE

Preparation Process CB1: Preparation of a Concentrated Co-Polyamino Acid/Insulin Glargine Composition with a pH of 7.2 Following a Process Using Insulin Glargine in Liquid Form (in Solution) and a Co-Polyamino Acid in Liquid Form (in Solution).

Concentrated solutions of NaCl and zinc chloride are added to a stock solution of co-polyamino acid with a pH of 7.1 in order to reach the target concentrations in the final composition. An insulin glargine solution as described in example C1 is added to this co-polyamino acid solution. A turbidity appears. The pH is adjusted to pH 7.5 by adding concentrated NaOH and the solution is placed under static conditions at +40° C. until complete solubilization. The solution obtained is visually clear and left to cool to 20-25° C. The pH is adjusted to 7.2 by adding a hydrochloric acid solution.

According to preparation process CB1, co-polyamino acid/insulin glargine compositions were prepared with insulin glargine concentrations between 50 IU/mL and 200 IU/mL.

### EXAMPLE CC: COMPOSITIONS COMPRISING INSULIN GLARGINE AND INSULIN LISPRO AT PH 7.2

Preparation process CC0: Preparation of a co-polyamino acid/insulin glargine/insulin lispro with a pH of 7.2

A lispro solution as described in example C2 is added to the concentrated co-polyamino acid/insulin glargine composition with a pH of 7.2 as described in example CB1 and, if necessary, water. The quantity of excipients added is selected in order to obtain a 0-1 mM concentration of zinc chloride, 0-10 mM of sodium chloride, 35 mM of m-cresol and 230 mM of glycerin in the lispro co-polyamino acid/insulin glargine composition. The solution obtained is clear. If necessary the pH is adjusted to the target of 7.2 by adding hydrochloric acid or sodium hydroxide solutions.

The compositions are filtered (0.22 μm) and stored at 4° C.

TABLE 2
Compositions of insulin glargine and insulin lispro in the presence of co-polyamino acid.

| | | Concentration of | Glargine | Lispro | | | |
|---|---|---|---|---|---|---|---|
| | Co-polyamino | co-polyamino acid | insulin | insulin | [ZnCl_{2]} | [NaCl] | |
| Composition | [symbol] | [symbol] | (IU/mL) | [symbol] | (mM) | (mM) | Visual |
| CC1 | AB35 | 3.1 | 75 | 25 | 0.5 | — | clear |
| CC2 | AB35 | 2.1 | 75 | 25 | 0.5 | 10 | clear |
| CC3 | AB35 | 1.5 | 75 | 25 | 0.7 | 10 | clear |
| CC4 | AB35 | 1.8 | 75 | 25 | 0.7 | 5 | clear |
| CC5 | AB35 | 4.2 | 150 | 50 | 1 | 10 | clear |
| CC6 | AB36 | 2.5 | 75 | 25 | 0.7 | 5 | clear |
| CC7 | AB24 | 1.7 | 75 | 25 | 0.5 | 10 | clear |

[symbol]

indicates data missing or illegible when filed

Co-polyamino acids according to the invention are used to solubilize insulin glargine in the presence of lispro insulin with a neutral pH and lead to clear solutions.

### Part CD: Results

### Part CD1: Demonstrating the Physical Stability of the Compositions According to the Invention by Studying the Previously Prepared Compositions

CD1 protocol: Study of the physical stability of insulin glargine/lispro insulin co-polyamino acid compositions with a pH of 7.2.

At least 5 glass 3 mL cartridges filled with 1 mL of co-polyamino acid/insulin glargine/prandial insulin are placed in an oven set to 30° C. in static conditions. The cartridges are inspected visually twice a month in order to detect the appearance of visible particles or turbidity. This inspection is carried out according to the recommendations of the European pharmacopoeia (EP 2.9.20): the cartridges are subjected to a lighting of at least 2,000 Lux and are observed on a white background and a black background. The number of weeks of stability corresponds to the duration from which most cartridges have visible particles or are turbid. The results are shown in table 3.

TABLE 3
Physical stability of the compositions of the invention CC1, CC2, CC5 and CC6.

| | | | Glargine | | | | Stability |
|---|---|---|---|---|---|---|---|
| | | Concentration of | insulin | Lispro | [ZnCl₂] | [NaCl] | 30° C. |
| Composition | Co-polyamino | co-polyamino | (IU/mL) | insulin | (mM) | (mM) | (week) |
| CC1 | AB35 | 3.1 | 75 | 25 | 0.5 | — | >12 |
| CC2 | AB35 | 2.1 | 75 | 25 | 0.5 | 10 | >12 |
| CC5 | AB35 | 4.2 | 150 | 50 | 1 | 10 | >12 |
| CC-6 | AB36 | 2.5 | 75 | 25 | 0.7 | 51 | >8 |

Compositions CC1, CC2, CC5 and CC6 show good physical stability.

### Part D Pharmacokinetics

### D1: Protocol for Measuring the Pharmacokinetics of Insulin Glargine and Insulin Lispro Formulations.

Studies on dogs have been conducted in order to evaluate the pharmacokinetics of insulins after administration of a co-polyamino acid AB35/insulin glargine (150 IU/mL)/lispro insulin (50 IU/mL) composition.

The pharmacokinetic profiles of insulin glargine (sum of the circulating concentration of insulin glargine and its main metabolite M1) and lispro insulin were obtained for this composition.

Ten animals that were fasted for about 17.5 hours were subcutaneously injected with a dose of 0.68 U/kg insulin. Blood samples are taken during the 16 hours following administration to describe the pharmacokinetics of the insulins. The levels of glargine, of glargine-M1 and lispro are determined by a specific bioanalysis method.

The pharmacokinetic parameters determined are as follows:
AUC₀₋₁ₕ, AUC₀₋₂ₕ, AUC₁₀₋₁₆ₕ, AUC₁₃₋₁₆ₕ corresponding to the area under the curve of insulin glargine concentrations (and its metabolite M1) as a function of time between 0 and 1 hour, 0 and 2 hours, 10 and 16 hours and 13 and 16 hours respectively post-administration;
AUC₀₋₃₀ₘᵢₙ, AUC₀₋₁ₕ, AUC₈₋₁₆ₕ corresponding to the area under the curve of insulin lispro concentrations as a function of time between 0 and 0.5 hours, 0 and 1 hour respectively and 8 and 16 hours post-administration;
AUCₗₐₛₜ corresponding to the surface under the curve between time 0 and the last measurement time of the subject.

Table 4 below shows different pharmacokinetic parameters for insulin glargine and insulin lispro.

TABLE 4
Average pharmacokinetic parameters (average ratio) of composition CC5 comprising
co-polyamino acid AB35/insulin glargine 150 U/mL/lispro insulin 50 U/mL.

| | Glargine insulin 150 IU/mL | | | | Lispro insulin 50 IU/mL | | |
|---|---|---|---|---|---|---|---|
| | AUC_{0-1 h}/ | AUC_{0-2 h}/ | AUC_{10-16 h}/ | AUC_{13-16 h}/ | | AUC_{0-1 h}/ | AUC_{8-16 h}/ |
| | AUCₗₐₛₜ | AUCₗₐₛₜ | AUCₗₐₛₜ | AUCₗₐₛₜ | AUC₀₋_{30 min}/ | AUCₗₐₛₜ | AUCₗₐₛₜ |
| | (%) | (%) | (%) | (%) | AUCₗₐₛₜ (%) | (%) | (%) |
| CC5 | 22.0 | 34.7 | 18.8 | 8.0 | 22.8 | 48.0 | 1.1 |

The results obtained indicate that, on one hand, the glargine component of the formulation is absorbed rapidly (AUC₀₋₁ₕ and AUC₀₋₂ₕ) while retaining its basal character with significant coverage on the terminal part of the observation time (AUC₁₀₋₁₆ₕ and AUC₁₃₋₁₆ₕ).

On the other hand, the lispro component is rapidly absorbed (AUC₀₋₃₀ₘᵢₙ and AUC₀₋₁ₕ) and retains its prandial character. In fact, lispro is no longer observed after 8 hours (AUC₈₋₁₆ₕ).

1. Composition in the form of an injectable aqueous solution, the pH of which is comprised from 6.0 to 8.0, comprising at least:
a) insulin glargine,
b) a co-polyamino acid bearing carboxylate charges and hydrophobic Hy radicals, the said co-polyamino acid consisting of glutamic or aspartic units and said hydrophobic Hy radicals from following formula I below:
*GpRᵣGpAₐGpC)ₚ Formula I
wherein
GpR is a radical according to formulas II, I′ or II″: 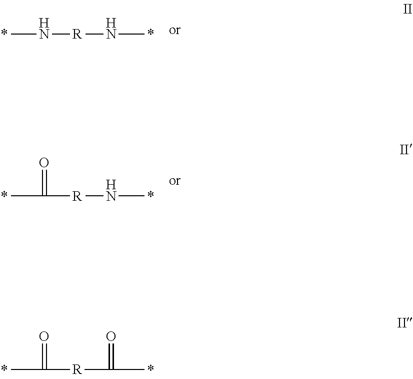
GpA is a radical according to formulas III or III′: 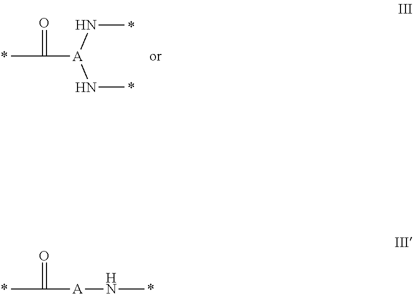
GpC is a radical according to formula IV: 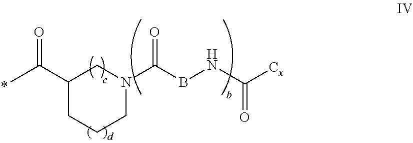
indicate the attachment sites of the various groups;
a is an integer equal to 0 or 1;
b is an integer equal to 0 or 1;
p is an integer equal to 1 or 2 and
if p is equal to 1 then a is equal to 0 or 1 and GpA is a radical according to formula III′ and,
if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
if r is equal to 1 or 2, then the hydrophobic radical according to formula I is bound to the co-polyamino acid:
through a covalent bond between a nitrogen atom of the hydrophobic radial and a carbonyl of the copolyamino acid, thus forming an amide function originating from the reaction of an amine function of the precursor of the hydrophobic radical and an acid function borne by the precursor of the co-polyamino acid or
through a covalent bond between a carbonyl of the hydrophobic radical and a nitrogen atom in N-terminal position of the co-polyamino acid, thus forming an amide function originating from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms:
a linear or branched divalent alkyl radical, comprising from 2 to 12 carbon atoms if GpR is a radical according to formula II or from 1 to 11 carbon atoms if GpR is a radical according to formula II′ or II″;
a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
an unsubstituted ether or polyether radical comprising from 4 to 14 carbon atoms and from 1 to 5 oxygen atoms;
A is a radical chosen from the group consisting of an unsubstituted ether or polyether radical comprising from 4 to 14 carbon atoms and from 1 to 5 oxygen atoms or a linear or branched alkyl radical comprising from 1 to 8 carbon atoms and optionally substituted by a radical derived from a saturated, unsaturated or aromatic ring;
B is a linear or branched alkyl radical, optionally comprising an aromatic ring, comprising from 1 to 9 carbon atoms;
Cₓ is a linear or branched monovalent alkyl radical, optionally comprising a cyclic part, wherein x indicates the number of carbon atoms and:
if p is equal to 1, x is comprised from 9 to 25 (9≤x≤25):
if p is equal to 2, x is comprised from 9 to 15 (9≤x≤15),
the ratio i between the number of hydrophobic radicals and the number of glutamic or aspartic units being between 0<i≤0.5;
when several hydrophobic radicals are carried by a co-polyamino acid they are therefore, identical or different;
the degree of polymerization DP of glutamic or aspartic units is comprised from 5 to 250;
the free acid functions being in the form of an alkaline cation salt chosen from the group consisting of Na⁺ and K⁺,
the said composition comprising at least one ion species chosen from the group of anions, cations and/or zwitterions.

2. Composition according to claim 1, wherein it also includes a prandial insulin and/or a gastrointestinal hormone.

3. Composition according to claim 1, wherein said hydrophobic radicals are selected from the hydrophobic radicals according to formula I wherein p=1, represented by formula V below:
*GpRᵣGpAₐGpC Formula V
GpR, GpA, GpC, r and a as defined in claim 1.

4. Composition according to claim 1, wherein the said hydrophobic radicals are selected from the hydrophobic radicals according to formula I wherein a=1 and p=2, represented by formula VI below:
*GpRᵣGpAGpC)₂ Formula VI
wherein
GpR, GpA, GpC, r and a as defined in claim 1.

5. Composition according to claim 1, wherein the co-polyamino acid bearing carboxylate charges and hydrophobic radicals is selected from the co-polyamino acids according to formula VII below: 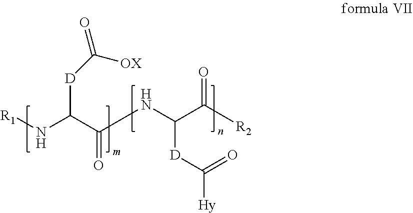
wherein,
D represents, independently, either a —CH₂— group (aspartic unit) or a —CH₂—CH₂— group (glutamic unit);
Hy is a hydrophobic radical selected from hydrophobic radicals according to formula I, V or VI;
R₁ is a hydrophobic radical selected from radicals according to formula I, V or VI wherein r=0, r=1 or r=2 or a radical chosen from the group consisting of a H, a linear C2-C10 acyl group, a branched C3 to C10 acyl group, benzyl, a terminal “amino acid” unit, and a pyroglutamate;
R₂ is a hydrophobic radical selected from hydrophobic radicals according to formula I, V or VI, or a radical —NR′R″, R′ and R″ identical or different chosen from the group consisting of H, linear or branched alkyls or cyclical in C2 to C10, benzyl and said R′ and R″ alkyls may form together one or more saturated carbon rings, unsaturated and/or aromatic and/or may contain heteroatoms, chosen from the group consisting of O, N and S;
X represents an H or a cationic entity chosen from the group consisting of metal cations;
n+m represents the degree of polymerization DP of the co-polyamino acid, namely the average number of monomeric units per co-polyamino acid chain and 5≤n+m≤250,
said co-polyamino acid containing at least one -Hy radical.

6. Composition according to claim 4, wherein the co-polyamino acid bearing carboxylate charges and hydrophobic radicals is selected from the co-polyamino acids according to formulas VII, wherein R₁=R′₁ and R₂=R′₂, according to formula VIIa below: 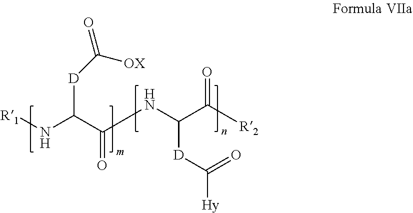
wherein,
n+m represents the degree of polymerization DP of the co-polyamino acid, namely the average number of monomeric units per co-polyamino acid chain and 5≤n+m≤250, X represents an H or a cationic entity chosen from the group consisting of metal cations, D represents, independently, either a —CH₂— group (aspartic unit) or a —CH₂—CH₂— group (glutamic unit), and Hy is a hydrophobic radical selected from hydrophobic radicals according to formula I, V or VI;
R′₁ is a radical chosen from the group consisting of H, linear C2 to C10 acyl group, branched C3 to C10 acyl group, benzyl, terminal amino acid unit and pyroglutamate;
R′₂ is a radical —NR′R″, R′ and R″ identical or different chosen from the group consisting of H, linear or branched or cyclic C2 to C10 alkyls, benzyl and said R′ and R″ alkyls which can form together one or more saturated, unsaturated and/or aromatic carbon rings and/or which may contain heteroatoms chosen from the group consisting of O, N and S.

7. Composition according to claim 4, wherein the co-polyamino acid bearing carboxylate charges and hydrophobic radicals is selected from the co-polyamino acids according to formula VII wherein n=0 according to formula VIIb below: 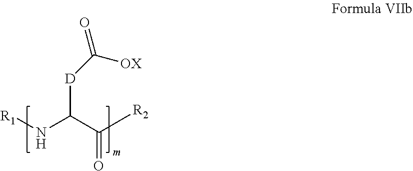
wherein n+m represents the degree of polymerization DP of the co-polyamino acid, namely the average number of monomeric units per co-polyamino acid chain and 5≤n+m≤250, X represents an H or a cationic entity chosen from the group consisting of metal cations, D represents, independently, either a —CH₂— group (aspartic unit) or a —CH₂—CH₂— group (glutamic unit), R₁ is a hydrophobic radical selected from radicals according to formula I, V or VI wherein r=0, r=1 or r=2 or a radical chosen from the group consisting of a H, a linear C2-C10 acyl group, a branched C3 to C10 acyl group, benzyl, a terminal “amino acid” unit, and a pyroglutamate, and R₂ is a hydrophobic radical selected from hydrophobic radicals according to formula I, V or VI, or a radical —NR′R″, R′ and R″ identical or different chosen from the group consisting of H, linear or branched alkyls or cyclical in C2 to C10, benzyl and said R′ and R″ alkyls may form together one or more saturated carbon rings, unsaturated and/or aromatic and/or may contain heteroatoms, chosen from the group consisting of O, N and S, and at least R₁ or R₂ is a hydrophobic radical according to formula I, V or VI.

8. Composition according to claim 1, wherein insulin glargine whose isoelectric point is comprised from 5.8 to 8.5 is insulin glargine.

9. Composition according to claim 1, wherein the content of insulin glargine whose isoelectric point is comprised from 5.8 to 8.5 is comprised from 40 to 500 U/mL.

10. Composition according to claim 1, wherein the weight ratio between insulin glargine and the co-polyamino acid, or co-polyamino acid/insulin glargine, is comprised from 0.2 to 8.

11. Composition according to claim 1, wherein the concentration of co-polyamino acid bearing carboxylate charges and hydrophobic radicals is at most 60 mg/mL.

12. Composition according to claim 1, wherein it further comprises a prandial insulin.

13. Composition according to claim 1, wherein the proportions between insulin glargine and prandial insulin are in percentages of 25/75, 30/70, 40/60, 50/50, 60/40, 63/37, 70/30, 75/25, 80/20, 83/17 or 90/10.

14. Composition according to claim 1, wherein it further comprises a gastrointestinal hormone.

15. Co-polyamino acid bearing carboxylate charges and hydrophobic radicals Hy, said co-polyamino acid consisting of glutamic or aspartic units and said hydrophobic radicals Hy selected from the radicals according to formula I as defined below:
*GpRᵣGpAₐGpC) Formula I
wherein
GpR is a radical according to formulas II, II′ or II″: 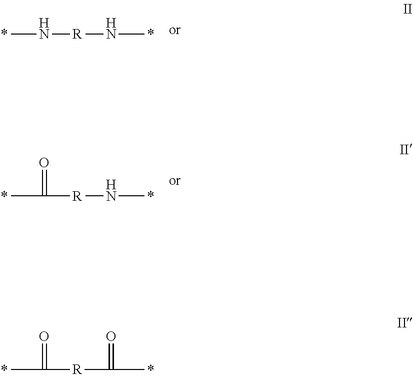
GpA is a radical according to formulas III or III′: 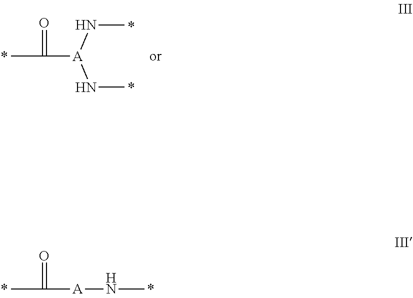
GpC is a radical according to formula IV: 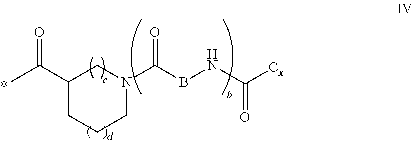
indicate the attachment sites of the various groups;
a is an integer equal to 0 or 1;
b is an integer equal to 0 or 1;
p is an integer equal to 1 or 2 and
if p is equal to 1 then a is equal to 0 or 1 and GpA is a radical according to formula III′ and,
if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
if r is equal to 1 or 2, then the hydrophobic radical according to formula I is bound to the co-polyamino acid:
through a covalent bond between a nitrogen atom of the hydrophobic radial and a carbonyl of the copolyamino acid, thus forming an amide function originating from the reaction of an amine function of the precursor of the hydrophobic radical and an acid function borne by the precursor of the co-polyamino acid or
through a covalent bond between a carbonyl of the hydrophobic radical and a nitrogen atom in N-terminal position of the co-polyamino acid, thus forming an amide function originating from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms; more precisely, R is a radical chosen from the group consisting of:
a linear or branched divalent alkyl radical, comprising from 2 to 12 carbon atoms if GpR is a radical according to formula II or from 1 to 11 carbon atoms if GpR is a radical according to formula II′ or II″;
a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
an unsubstituted ether or polyether radical comprising from 4 to 14 carbon atoms and from 1 to 5 oxygen atoms;
A is a radical chosen from the group consisting of an unsubstituted ether or polyether radical comprising from 4 to 14 carbon atoms and from 1 to 5 oxygen atoms or a linear or branched alkyl radical comprising from 1 to 8 carbon atoms and optionally substituted by a radical derived from a saturated, unsaturated or aromatic ring;
B is a linear or branched alkyl radical, optionally comprising an aromatic ring, comprising from 1 to 9 carbon atoms;
Cₓ is a linear or branched monovalent alkyl radical, optionally comprising a cyclic part, wherein x indicates the number of carbon atoms and:
if p is equal to 1, x is comprised from 9 to 25 (9≤x≤25):
if p is equal to 2, x is comprised from 9 to 15 (9≤x≤15),
the ratio i between the number of hydrophobic radicals and the number of glutamic or aspartic units being between 0<i≤0.5;
when several hydrophobic radicals are carried by a co-polyamino acid they are therefore, identical or different;
the degree of polymerization DP of glutamic or aspartic units is comprised from 5 to 250;
the free acid functions being in the form of an alkaline cation salt chosen from the group consisting of Na⁺ and K⁺

16. Hy ‘precursor of the hydrophobic radical Hy according to formula I as defined below:
HGpRᵣGpAₐGpC)ₚ Formula I′
wherein
GpR is a radical according to formulas II, I′ or II″: 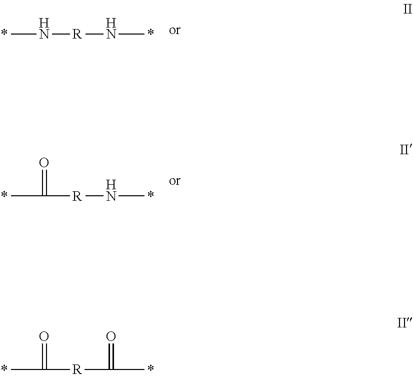
GpA is a radical according to formulas III or III′: 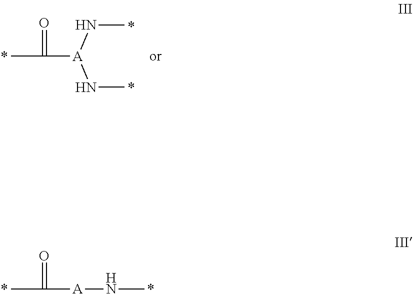
GpC is a radical according to formula IV: 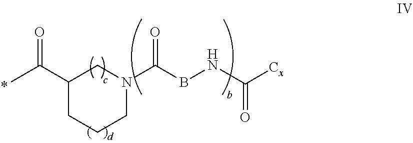
indicate the attachment sites of the various groups;
a is an integer equal to 0 or 1;
b is an integer equal to 0 or 1;
p is an integer equal to 1 or 2 and
if p is equal to 1 then a is equal to 0 or 1 and GpA is a radical according to formula III′ and,
if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
if r is equal to 1 or 2, then the hydrophobic radical according to formula I is bound to the co-polyamino acid:
through a covalent bond between a nitrogen atom of the hydrophobic radial and a carbonyl of the copolyamino acid, thus forming an amide function originating from the reaction of an amine function of the precursor of the hydrophobic radical and an acid function borne by the precursor of the co-polyamino acid or
through a covalent bond between a carbonyl of the hydrophobic radical and a nitrogen atom in N-terminal position of the co-polyamino acid, thus forming an amide function originating from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH₂ functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms; more precisely, R is a radical chosen from the group consisting of:
a linear or branched divalent alkyl radical, comprising from 2 to 12 carbon atoms if GpR is a radical according to formula II or from 1 to 11 carbon atoms if GpR is a radical according to formula II′ or II″;
a divalent alkyl radical, linear or branched, comprising from 2 to 11 carbon atoms if GpR is a radical according to formula II or from 1 to 11 carbon atoms if GpR is a radical according to formula II′ or II″, said radical alkyl bearing one or more —CONH₂ functions, and
an unsubstituted ether or polyether radical comprising from 4 to 14 carbon atoms and from 1 to 5 oxygen atoms;
A is a radical chosen from the group consisting of an unsubstituted ether or polyether radical comprising from 4 to 14 carbon atoms and from 1 to 5 oxygen atoms or a linear or branched alkyl radical comprising from 1 to 8 carbon atoms and optionally substituted by a radical derived from a saturated, unsaturated or aromatic ring;
B is a linear or branched alkyl radical, optionally comprising an aromatic ring, comprising from 1 to 9 carbon atoms;
Cₓ is a linear or branched monovalent alkyl radical, optionally comprising a cyclic part, wherein x indicates the number of carbon atoms and:
if p is equal to 1, x is comprised from 9 to 25 (9≤x≤25):
if p is equal to 2, x is comprised from 9 to 15 (9≤x≤15),
the ratio i between the number of hydrophobic radicals and the number of glutamic or aspartic units being between 0<i<0.5;
when several hydrophobic radicals are carried by a co-polyamino acid they are therefore, identical or different;
the degree of polymerization DP of glutamic or aspartic units is comprised from 5 to 250;
the free acid functions being in the form of an alkaline cation salt chosen from the group consisting of Na⁺ and K⁺.

17. (canceled)
